(19) United States
(12) Patent Application Publication
(10) Pub. No.: US 2015/0051452 A1
(43) Pub. Date: Feb. 19, 2015

(54) APPARATUS, METHOD AND COMPUTER-ACCESSIBLE MEDIUM FOR TRANSFORM ANALYSIS OF BIOMEDICAL DATA

(71) Applicant: EDWARD J. CIACCIO, Cherry Hill, NJ (US)

(72) Inventor: EDWARD J. CIACCIO, Cherry Hill, NJ (US)

(73) Assignee: THE TRUSTEES OF COLUMBIA UNIVERSITY IN THE CITY OF NEW YORK, New York, NY (US)

(21) Appl. No.: 14/467,300

(22) Filed: Aug. 25, 2014

Related U.S. Application Data
(63) Continuation-in-part of application No. 14/114,038, filed on Jan. 8, 2014.
(60) Provisional application No. 61/479,168, filed on Apr. 26, 2011.
(60) Provisional application No. 61/869,263, filed on Aug. 23, 2013.
(60) Provisional application No. 61/886,903, filed on Oct. 4, 2013.
(60) Provisional application No. 61/887,521, filed on Oct. 7, 2013.
(60) Provisional application No. 61/973,437, filed on Apr. 1, 2014.

(51) Int. Cl. A61B 5/00; A61B 5/0205; A61B 1/00; G06F 17/14; A61B 1/04

(52) U.S. Cl. CPC A61B 5/7257; G06F 17/14; A61B 5/0084; A61B 5/7203; A61B 1/041; A61B 1/00009; A61B 5/4255; A61B 5/7264; A61B 5/0205; A61B 5/02416
USPC 600/301; 600/300; 600/476; 600/479; 600/407

(57) ABSTRACT

Exemplary method, computer-readable medium and system can be provided for generating at least one information associated with at least one signal and/or data received from at least one structure. For example, it is possible to determine at least one basis based on a combination of a plurality of portions of the signal(s) and/or the data. It is also possible to generate the information(s) as a function of the basis.

### CROSS-REFERENCE TO RELATED APPLICATIONS

The present application is a continuation-in-part of U.S. National Phase patent application Ser. No. 14/114,038 filed on Jan. 8, 2014, and relates to and claims priority from International Application No. PCT/US2012/035154 filed on Apr. 26, 2012, U.S. Provisional Patent Application No. 61/479,168 filed on Apr. 26, 2011, U.S. Provisional Patent Application No. 61/869,263 filed on Aug. 23, 2013, U.S. Provisional Patent Application No. 61/886,903 filed on Oct. 4, 2013, U.S. Provisional Patent Application No. 61/887,521 filed on Oct. 7, 2013, and U.S. Provisional Patent Application No. 61/973,437 filed on Apr. 1, 2014, the entire disclosures of which are incorporated herein by reference

### FIELD OF THE DISCLOSURE

The present disclosure relates generally to the analysis of biomedical data, and more specifically, relates to exemplary embodiments of apparatus, method, and computer-readable medium for performing an ensemble transform analysis of biomedical signals

### BACKGROUND INFORMATION

Representation of independent biophysical sources using Fourier analysis can be inefficient because the basis can typically be sinusoidal and general. When complex fractionated atrial electrograms (“CFAE”) are acquired during atrial fibrillation (“AF”), the electrogram morphology typically can depend on a mix of distinct non-sinusoidal generators.

Transforms that use a general basis, similar to a Fourier analysis, can be inefficient for representation of independent biophysical sources, or drivers, unless these happen to be generated by sinusoidal functions. In contrast, transforms that use data-driven bases can be efficacious for distinguishing uncorrelated signal components generated by independent drivers, if the morphology can be reproduced in the basis. For example, the Fukunaga-Koontz transform can be useful to discern two independent sources in cardiac electrogram data by separating correlated versus uncorrelated components of the variance (e.g., second central moment). (See, e.g., Reference 1). Development of a data-driven basis and transform that can utilize the ensemble average (e.g., first central moment) can be desirable to detect the actual signal morphologic components originating from distinct sources. This can be useful, for example, in the analysis of CFAE, (see, e.g., Reference 2), which can likely be formed by multiple independent generators (e.g., focal areas of high frequency and/or reentrant circuits). (See, e.g., References 3-6). It can also be possible that the ensemble averaging can be done by correlating portions of signals rather than by combining portions of signals by averaging, weighted averaging or some other statistical function.

Currently, CFAE can be quantified using the dominant frequency (“DF”), which can be defined as the largest spectral component over the physiologic range of electrical activation rate (e.g., about 2-10 Hz). (See, e.g., Reference 7). A calculation of the DF of CFAE using ensemble averaging has typically been done (see, e.g., References 17 and 18). The dominant frequency can typically be calculated by bandpass filtering the CFAE, rectification and low pass filtering of the result, followed by Fourier power spectral analysis. (See, e.g., References 8 and 9). However, the filtering process can distort important signal components and the method may not typically be robust to phase noise. (See, e.g., References 10-13). Moreover, signal morphologic components arising from each generator may not typically be readily apparent in the sinusoidal basis.

Accordingly, the identification of these generators using efficient methods of representation and comparison can be useful for targeting catheter ablation sites to prevent arrhythmia reinduction. For example, a development of an improved estimate of independent generator frequency and morphologic characteristics can potentially be useful, for example, to target abnormal atrial tissue for catheter ablation (see, e.g., Reference 14), particularly for persistent AF cases. (See, e.g., References 15 and 16).

Celiac disease is typically an autoimmune disease which can manifest as villous atrophy in the small intestinal lining or mucosa (see, e.g., Reference 9A and 10A). The result can be fissuring of the mucosal surface, as well as a scalloped appearance of the small intestinal mucosal folds, both of which can result in an abnormality that can often be observable by eye in acquired videocapsule images. Upon quantitative analysis, it can be shown that the DP of a sequential series of videocapsule images can be significantly longer in celiac disease as compared to control patients, possibly indicating decreased small intestinal motility (see, e.g., Reference 8A). Furthermore, the relationship between DP and small intestinal transit time can be approximately linear for both celiacs and controls (see, e.g., Reference 8A). Thus, frequency analysis using videocapsule image frames can be potentially useful for clinical diagnostics.

Isolation of an electrical activity in the pulmonary veins (“PV”) can be a first step to prevent AF when drug therapy fails. (See, e.g., References 38 and 39). This technique works reasonably well in patients with paroxysmal AF. (See, e.g., Reference 40). Ablation of other areas of the left atrium can eliminate arrhythmogenic sites, stop AF, and prevent its recurrence in both paroxysmal (see, e.g., Reference 41) and persistent AF substrates (see, e.g., Reference 42), although additional procedures can be used, particularly in cases of persistent AF. (See, e.g., Reference 43). Sites with complex fractionated atrial electrograms (“CFAE”) have been proposed as arrhythmogenic targets (see, e.g., Reference 44) for catheter ablation. CFAE can be defined as electrograms with continuous electrical activity without isoelectric segment approximately greater than 50 ms, or activity with period approximately less than 100 ms. (See, e.g., Reference 44). Since areas of the left atrium containing CFAE can be extensive, ablating all of the CFAE sites in AF patients can markedly increase procedure time, and can possibly cause morbidity. Thus, there can be a need to characterize CFAE by quantitative means for detection of specific characteristics that can be helpful toward deciding what sites to target for ablation.

The DF can be a ubiquitous tool to quantitatively characterize CFAE during and after the electrophysiologic study of AF patients. (See, e.g., References 45 and 46). It can be defined as the largest fundamental component of the frequency spectrum within the electrophysiologic range of interest. (See, e.g., Reference 45). The DF can be computed in early work by preprocessing the signal with a bandpass filter, followed by rectification and low-pass filtering. (See, e.g., References 47 and 48). In more recent publications, the preprocessing stage can be eliminated to prevent signal distortion. (See, e.g., Reference 49). Spectral estimators, which can be more robust to random and phase noise as compared with Fourier analysis, have also recently been devised. (See, e.g., Reference 50). Furthermore, spectral parameters in addition to the DF have been developed as additional measures of the frequency characteristics of CFAE. (See, e.g., Reference 50). These can include the dominant amplitude (“DA”), which can be defined as the amplitude of the DF spectral peak. The parameter can be related to the power under the dominant peak, but does not need guestimation of the start and end of the peak. (See, e.g., Reference 50). A smaller value of DA can indicate less power in the dominant peak, more power in the background level, and therefore greater complexity of electrical activity. The mean and standard deviation in the spectral profile (“MP” and “SP”) have been recently described. (See, e.g., Reference 50).

Unlike measurements of features related to spectral power, these parameters do not need guestimation concerning the dominant peak or its harmonics. A greater value of MP and/or SP can indicate a higher background level and, therefore, greater complexity of electrical activity. Prior findings of lesser DA and greater MP and SP in paroxysmal AF (see, e.g., Reference 50) can suggest that there can be a greater level of instability in the atrial activation pattern during paroxysmal as compared with persistent AF. This finding can represent a first step in quantitatively characterizing differences in the substrate between these AF types.

Recent work has also found morphologic differences in the time series of these signals to be useful in characterizing paroxysmal versus persistent AF. (See, e.g., References 51 and 52). When the morphologic descriptors can be more variable, it can be indicative of increasing instability in the electrical activation pattern from which the extracellular signals can be formed. More variable morphologic descriptors, and therefore, increased instability, can again be found in paroxysmal AF as compared with persistent AF recordings. (See, e.g., References 51 and 52). Therefore, both frequency and morphologic measurements can be suggestive that significant differences in the electrical activation pattern can exist in paroxysmal versus persistent AF, and that the persistent AF recordings can be less variable and more stable, possible due to presence of relatively intransigent drivers.

Accordingly, there can be a need to address and/or overcome at least some of the above described deficiencies and issues.

### SUMMARY OF EXEMPLARY EMBODIMENTS

These and other deficiencies can be addressed with the exemplary embodiments of the present disclosure.

For example, according to certain exemplary embodiments of the present disclosure, apparatus, methods and computer-readable medium can be provided for analyzing biomedical data using a new transform which does not distort analyzed signals, and can be robust to phase noise, for calculation of the DF, and the identification of independent generator frequency and morphology in CFAE. Exemplary derivations of the exemplary transform procedure, according to certain exemplary embodiments of the present disclosure, can also be implemented. Exemplary embodiments of the present disclosure can also provide comparisons of the exemplary transform to Fourier analysis to measure the DF of CFAE, and the robustness of each method of DF measurement when random noise can be added to the signal. Additionally, the frequencies of simulated drivers embedded in CFAE in the presence of phase noise and interference can be detected with each exemplary procedure. Further, correspondence(s) can be shown between basis vectors of the highest power derived from the new transform, versus actual CFAE morphology and synthesized drivers.

According to further exemplary embodiments of the present disclosure, apparatus, method and computer-readable medium can be provided for an evaluation of CFAE signals. For example, the ensemble average of signal segments can be used to construct a data-driven basis, and it can be shown to have significant advantages over Fourier analysis for correct prediction of the DF of independent drivers in presence of phase noise and interference, as well as for representation of CFAE signals in general, and the distinctive morphologic components associated with each independent synthetic driver that can be tested. The exemplary transform can have possible applications for targeting drivers of atrial fibrillation during clinical catheter ablation to prevent reinduction of the arrhythmia, as well as for improved understanding of the mechanisms by which paroxysmal and persistent AF can be initiated and maintained.

According to additional exemplary embodiments of the present disclosure, synthetic image sequences can be generated with spatiotemporal phase noise, random noise, and air bubbles imposed, to validate the measurement of the DP in videocapsule image series. Instead of using average image brightness level for spectral analysis, the image frames can be analyzed pixel-by-pixel, which can increase robustness to the presence of extraneous features and noise. Because of the smoothing effect of analyzing the spectra from many pixels and taking the mean, the repetition rate of a synthesized sequence of images can be detectable even at high noise level.

According to yet further exemplary embodiments of the present disclosure, apparatus, methods and computer readable medium can be provided for a robust spectral analysis of videocapsule images of celiac diseases. For example, videocapsule endoscopy can be useful to detect mechanical rhythms of the small intestinal lumen via the dominant period (“DP”) spectral calculation. However, noise and air bubbles can obscure image features, and can mask rhythms. Fourier versus ensemble averaging spectral analysis can be used to detect simulated periodicity in small intestinal images. According to certain exemplary embodiments of the present disclosure, for example, about ten-to-twenty sequential image frames sampled at, for example, about 2 frames/second can be extracted from each of 10 video clips obtained from 10 celiac disease patients (e.g., about 576×576 pixel resolution). These frames can be repeated to create a synthesized sequence about 200 frames in length, typical for quantitative analysis of video clips. Random noise, spatiotemporal phase shift and imposition of air bubble frames can be used for sequence degradation. Power spectra can be then computed pixel-by-pixel from the brightness levels over 200 image frames.

For example, the tallest peak in the mean power spectrum calculated from the 576×576 pixel-level spectra can be taken as the DP. The absolute difference between the actual DP based on repetition of the frames sequence, versus the estimated value from spectral analysis, can be tabulated, as can the speed of computation for Fourier versus ensemble averaging methods. For the additive noise levels, for example, the mean absolute difference between estimated versus actual DP can be, for example, about 0.0547±0.0688 Hz for Fourier versus about 0.0031±0.0127 Hz for ensemble (e.g., p<0.001 in mean and standard deviation). The mean time for computing about a 331,776 pixel spectra per video clip can be, for example, about 12.31±0.01 s for Fourier versus about 4.86±0.01 s for ensemble (e.g., p<0.001). Ensemble spectral analysis according to certain exemplary embodiments of the present disclosure can be robust to additive noise and spatiotemporal jitter, and useful for rapid DP calculation in videocapsule image series.

According to further exemplary embodiments of the present disclosure, method, computer-readable medium and system can be provided for generating information associated with a signal(s) and/or data received from a structure(s). For example, it can be possible to determine a basis based on a combination of a plurality of portions of the signal(s) and/or the data. It can also be possible to generate the information(s) as a function of the basis.

In one exemplary embodiment, the combination can include a summation, an average, a weighted average and/or a statistical representation. The summation can include a summation of a plurality of segments of the signal(s) or the data. The generation of the information can include an application of a transform relating the combination a frequency(s) of the signal(s) so as to generate a power spectrum. The signal(s) or the data can include a video-capsule image associated with one of a celiac disease or a cardiac signal as obtained during atrial fibrillation. The information can include a dominant frequency, a dominant period, a mean and/or a standard deviation in a power spectral profile.

It is possible to qualify a characteristic(s) associated with the signal(s) or the data based on the transform, a noise, an interference and/or an artifact in generating a reconstruction of the signal(s) based on the transform. It is also possible to increase a frequency resolution for a given time period of the signal(s) or the data based on the transform. Further, it can be possible to cause a recognition of a source pattern of the signal(s) or the data based on the transform. The signal(s) or the data can be an image.

These and other objects, features and advantages of the present disclosure will become apparent upon reading the following detailed description of exemplary embodiments of the present disclosure, when taken in conjunction with the appended drawings and claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1D are exemplary graphs of exemplary power spectrum constructions according to an exemplary embodiment of the present disclosure;

FIGS. 2A-2D are exemplary graphs of exemplary synthetic drivers used for an exemplary spectral analysis and reconstruction according to an exemplary embodiment of the present disclosure;

FIGS. 3A-3D are exemplary graphs of exemplary average spectra for the exemplary simulated drivers according to an exemplary embodiment of the present disclosure;

FIGS. 4A-4D are exemplary illustrations of exemplary normalized inner product for 481 basis vectors with magnitude 0 at the bottom right and magnitude 1 at top according to an exemplary embodiments of the present disclosure;

FIG. 5A is an exemplary graph of an exemplary Fourier spectrum;

FIG. 5B is an exemplary graph of an exemplary ensemble spectrum according to an exemplary embodiment of the present disclosure;

FIGS. 6A-6D are exemplary graphs of exemplary ensemble basis vectors constructed from a synthesized signal according to an exemplary embodiment of the present disclosure;

FIG. 7A is an exemplary graph of exemplary CFAE from a paroxysmal AF patient;

FIG. 7B is an exemplary graph of exemplary CFAE with random noise;

FIGS. 7C and 7D are exemplary graphs of Fourier power spectrums for the signals shown in FIGS. 7A and 7B, respectively;

FIGS. 7E and 7F are graphs of exemplary ensemble power spectrums for the signals shown in FIGS. 7A and 7B, respectively, according to an exemplary embodiment of the present disclosure;

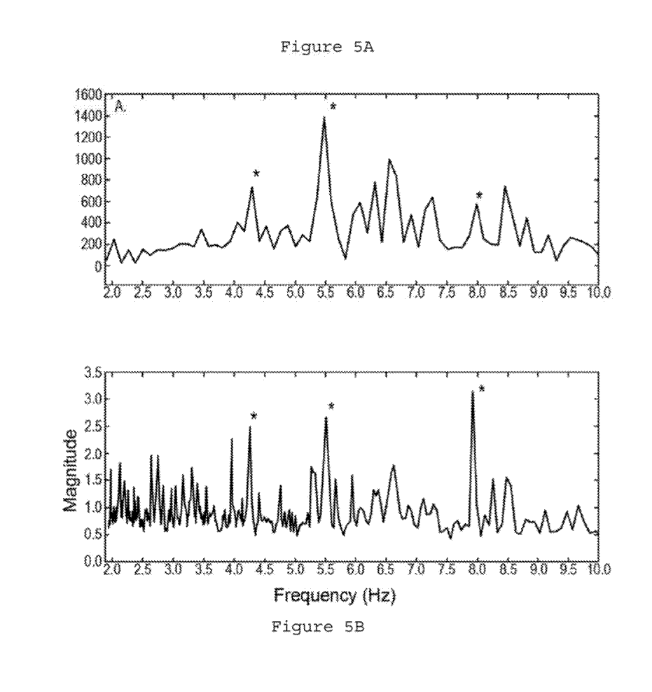

FIGS. 5A and 8B are exemplary graphs showing exemplary CFAE reconstructions with 1 and with 10 basis vectors using Fourier analysis;

FIGS. 8C and 8D are exemplary graphs showing exemplary CFAE reconstructions with 1 and with 10 basis vectors using ensemble analysis according to an exemplary embodiment of the present disclosure;

FIGS. 9A-9C are exemplary graphs showing exemplary statistics of Fourier and ensemble average reconstruction error for real CFAE signals;

FIG. 10A is an exemplary graph of an exemplary effect of a transient on a CFAE signal;

FIG. 10B is an exemplary graph of an exemplary CFAE from left inferior pulmonary vein of a persistent AF patient;

FIG. 10C is an exemplary graph of an exemplary Fourier power spectrum of the signal shown in FIG. 10B;

FIG. 10D is an exemplary graph of an exemplary ensemble average power spectrum of the signal shown in FIG. 10B according to an exemplary embodiment of the present disclosure;

FIGS. 11A-11C are exemplary graphs of additional exemplary Fourier versus ensemble averaging power spectra with the transient added to CFAE signals;

FIGS. 12A-12F are exemplary videocapsule series images;

FIGS. 13A-13F are exemplary videocapsule series images;

FIGS. 14A-14D are exemplary graphs of exemplary Fourier power spectra;

FIGS. 15A-15D are graphs of exemplary ensemble power spectra according to an exemplary embodiment of the present disclosure;

FIGS. 16A-16D are exemplary graphs of exemplary Fourier power spectra;

FIGS. 17A-17D are exemplary graphs of exemplary ensemble power spectra according to an exemplary embodiment of the present disclosure;

FIG. 18 is an exemplary illustration of an exemplary block diagram of an exemplary system in accordance with exemplary embodiment of the present disclosure;

FIGS. 19A-19D are exemplary graphs of frequency spectra used for analysis for CFAE with two closely spaced frequency components at the low end of range A;

FIGS. 20A-20D are exemplary graphs of frequency spectra used for analysis of CFAE with synthetic frequency components spaced further apart than those of FIG. 19;

FIGS. 21A-21F are exemplary graphs of atrial electrograms used as patterns A and B to be detected in the set of 216 initial recording sequences;

FIGS. 22A-22D are exemplary graphs of transform coefficients when two patterns A and B are embedded in interference;

FIGS. 23A-23D are exemplary graphs of spectral signatures of pattern A and B computed from the basis vectors derived from the mean signal;

FIGS. 24A-24C are exemplary graphs of a Euclidean distance between the power spectrum of the mean from 216 recordings, and the spectral signatures of 214 individual recordings with interference added;

FIG. 25 is a flow diagram illustrating a method in accordance with an exemplary embodiment of the present disclosure;

FIG. 26 is an exemplary graph of the exemplary spectral estimator according to an exemplary embodiment of the present disclosure;

FIGS. 27A and 27B are exemplary graphs of exemplary CFAE according to an exemplary embodiment of the present disclosure;

FIGS. 28A-28D are further exemplary graphs of the exemplary CFAE according to an exemplary embodiment of the present disclosure;

FIGS. 29A and 29B are exemplary graphs of an exemplary repeating pattern added to an exemplary CFAE according to an exemplary embodiment of the present disclosure;

FIGS. 30A and 30B are exemplary graphs of exemplary synthetic geometric shapes used to test the exemplary spectral estimate and the exemplary discrete Fourier transform according to an exemplary embodiment of the present disclosure;

FIGS. 31A and 31B are exemplary graphs of exemplary trials of the exemplary spectral estimator according to an exemplary embodiment of the present disclosure;

FIGS. 32A-32D are exemplary graphs of examples of CFAE in persistent AF according to an exemplary embodiment of the present disclosure;

FIGS. 33A-33D are exemplary graphs of 1^{st} and 2^{nd} 8 s DMs for exemplary CFAE according to an exemplary embodiment of the present disclosure;

FIGS. 34A-34D are exemplary graphs of exemplary CFAE in paroxysmal AF according to an exemplary embodiment of the present disclosure;

FIGS. 35A-35D are exemplary graphs of ensemble averages at the DF for the exemplary CFAE according to an exemplary embodiment of the present disclosure;

FIGS. 36A-36D are exemplary graphs of exemplary power spectra according to an exemplary embodiment of the present disclosure;

FIGS. 37A and 37B are exemplary graphs of an exemplary classification based on the DM correlation coefficients according to an exemplary embodiment of the present disclosure;

FIGS. 38A-38D are exemplary graphs of exemplary spectral estimates according to an exemplary embodiment of the present disclosure;

FIGS. 39A-39D are exemplary graphs of exemplary spectras of the exemplary spectral estimation for small changes in analysis window locations according to an exemplary embodiment of the present disclosure;

FIGS. 40A-40D are exemplary graphs of exemplary spectras of the exemplary spectral estimation for large changes in analysis window locations according to an exemplary embodiment of the present disclosure;

FIG. 41 is an exemplary schematic diagram of a hardware implementation of the exemplary apparatus configured to provide a real-time estimation according to an exemplary embodiment of the present disclosure; and

FIGS. 42A and 42B are exemplary graphs illustrating a spectral magnitude of the real-time spectral estimator according to an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and appended claims.

### DETAILED DESCRIPTION OF EXEMPLARY EMBODIMENTS

FIG. 25 shows a flow diagram illustrating use of a method in accordance with an exemplary embodiment of the present application.

As shown in FIG. 25, in procedure 2501, a signal can be received. The signal can be (i) a CFAE signal, (ii) an image received from a video capsule camera, (iii) a biomedical signal and/or (iv) any form of data. In procedure 2502, a combination of a plurality of portions of the signal can be taken. The combination can be a summation, an average, a weighted average and/or any other statistical measurement. In procedure 2503, data driven basis vectors can be determined from the combination of the plurality of portions of the signal. In procedure 2504, information can be generated as a function of, or based on, the basis vectors determined from procedure 2503. Further, in procedure 2505, the information generated can be analyzed and can be used in biomedical procedures, although not limited thereto. Any of the exemplary procedures set forth and/or described herein can be performed by the exemplary system shown in FIG. 18, as also described below.

### Exemplary Transform Equations

An autocorrelation coefficient r_{φ} at lag φ can be given by the inner product of two mean-zero signal vectors, for example:

r_{φ}=1/Nx₀^{T}·x_{φ} (1)

where x0 and x_{φ} can be of length N and can be given by, for example:

x₀=[x(k)x(k+1) . . . x(k+N−1)]^{T} (2a)

x_{φ}=[x(k−φ))x(k−φ+1) . . . x(k−φ+N−1)]^{T} (2b)

and the vectors can be normalized, a priori, by scaling to unity variance. Suppose that lag can φ represent a segment of x₀ that can be w sample points long. Exemplary Eq. (1) can then be rewritten as, for example:

r_{w}=1/nwΣᵢ·s_{wi}^{T}·s_{wi+1}i=1,n (3)

where s_{w} can be segments of signal x₀ having length w, for example:

s_{wi}=[x(w·i+1),x(w·i+2), . . . x(w·i+w)]^{T} (4a)

s_{wi+1}=[x(w·(i+1)+1),x(w·(i+1)+2), . . . x(w(i+1)+w)]^{T} (4b)

and the number of signal segments can be, for example:

n=int(N/w) (5)

Based on these exemplary equations, the autocorrelation function for w can be described as a graph of the mean autocorrelation between successive signal segment pairs swi and swi+1 as given by exemplary Eq. (3), versus segment length w. The segment length can be converted to a frequency, for example:

f=sample rate/w (6)

which can reduce to 1/w when the sample rate can be, for example, 1 kHz, and the time units can be milliseconds. The peak in the autocorrelation function over, for example, a frequency range f1 to f2 (e.g., 1/w1 to 1/w2) that can be physiologic for electrical activation rate, has been used to estimate the DF in atrial electrograms. (See, e.g., References 19-21).

A more robust alternative for adapting the autocorrelation function to spectral analysis can use ensemble averaging. (See, e.g., References 17 and 18). The ensemble average vector e_{w} can be obtained by averaging the n successive mean zero segments of signal x, each segment being of length w, for example:

e_{w}=1/n·U_{w}·x (7a)

U_{w}=[I_{w}I_{w} . . . I_{w}] (7b)

where I_{w} can be w×w identity submatrices used to form the signal segments that can be extracted from x and summed. Thus, for example:

e_{w}1/nΣᵢ·s_{wi}i=1,n (8)

where s_{wi} can be as given in exemplary Eq. (4a). The power in the ensemble average can be described by, for example:

$$\begin{matrix}{P}_{w} = \frac{1}{w {\underline{e}}_{w}^{T} * {\underline{e}}_{w}} & ( 9 a ) \\ = \frac{1}{{n}^{2} w {\underline{x}}^{T} {U}_{w}^{T} {U}_{w} \underline{x}} & ( 9 b ) \\ = \frac{1}{{n}^{2} w {Σ}_{i} {Σ}_{j} {\underline{S}}_{wi}^{T} {\underline{s}}_{wj}} & ( 9 c )\end{matrix}$$

where exemplary Eqs. (9b) and (9c) can be formed by substituting exemplary Eqs. (7) and (8) into exemplary Eq. (9a), and i and j can be segment numbers from 1 to n. Exemplary Eq. (9c) can be similar to exemplary Eq. (3), except that instead of computing the autocorrelation between successive signal segment pairs s_{wi}, s_{wi+1} only (e.g., lag w), it can be computed between signal segments s_{wi}, s_{wj}. P_{w} can be therefore equivalent to computing the mean autocorrelation coefficient from n points in the autocorrelation function separated by lag w, for example, to averaging the autocorrelation coefficients at lags w, 2w, 3w, nw. However, to generate P_{w}, in this way rather than by using exemplary Eq. (9c), would typically require a sequence length 2N to convolve the signal with itself along its entire length, halving the time resolution and doubling the sequence length generally needed for analysis.

To generate an ensemble power spectrum, the root mean square (“RMS”) power has been used (see, e.g., References 17 and 18), for example:

P_{wRMS}=√(P_{w}) (10)

where sqrt can be the square root function and the units can be millivolts. The power spectrum can be displayed by plotting sqrt(n)·P_{RMS} versus frequency f as computed from exemplary Eq. (6). The sqrt(n) term can level the spectral baseline, which would otherwise decrease by 1/sqrt(n), the amount of noise falloff per number of summations n used for ensemble averaging. From exemplary Eqs. 5, (9c), and (10), the displayed RMS power can be written as, for example:

√[n·P_{wRMS]=√[}1/NΣᵢΣⱼs_{wi}^{T}s_{wj}] (11)

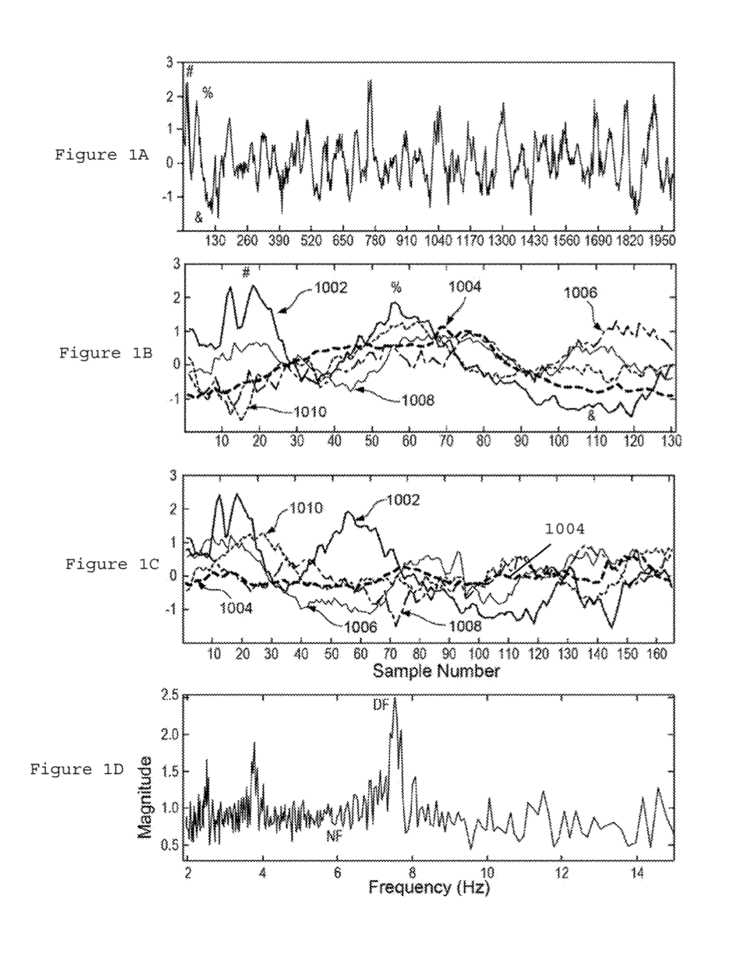

An example of ensemble average power spectrum construction is shown, for example, in the graphs of FIGS. 1A-1D. A typical CFAE from the left inferior pulmonary vein ostia during longstanding persistent AF is shown in FIG. 1A. The summing of the first four segments of width w=130 is shown in FIG. 1B, represented as elements 1002, 1010, 1008, and 1006, respectively The segmented traces often have peaks at similar locations (e.g., FIG. 1B). The ensemble average for the segments of width w=130 is shown as a dashed trace 1004 (e.g., FIG. 1B). It has similarities to segments 1-4 shown, and to many other CFAE segments having width w=130 (e.g., from Eq. (5), int(8192/130)=63 segments in total). For perspective, the x-axis scale is marked in intervals of 130 (e.g., FIG. 1A) with each scale mark representing the start of a new segment number. As shown in FIG. 1C, segments with width w=165 sample points are shown. The peaks may not be well-aligned, and the ensemble average, again shown as a dashed line 1004, can be of a lower amplitude than in FIG. 1B. Thus segments with width w=165 may not be well correlated.

The ensemble average calculation can be repeated for all segments w in the frequency range of interest, as given by exemplary Eq. (6) with a sampling rate of about 977 Hz. The RMS power in the ensemble average was then plotted using exemplary Eq. (11) (e.g., FIG. 1D). The DF occurs, for example, at about 7.52 Hz, corresponding to w=130 sample points (e.g., FIG. 1B). In contrast, the noise floor marked at ‘NF’, with f=5.92 Hz, occurs, for example, at w=165 sample points (e.g., FIG. 1C). The ensemble averaging spectrum thus can display correlated components as higher power, and can have more detail at the lower spectral range due to the w=1/f relationship (e.g., exemplary Eq. (6)). In addition, DF subharmonics can be pronounced.

The relation between the ensemble power spectrum and the Fourier power spectrum can be described as follows. Based upon the Wiener-Khinchin theorem, the Fourier transform of the autocorrelation function of a signal can be the power spectrum of that signal, for example:

$$\begin{matrix}S ( f ) = {Σ}_{φ} {r}_{xx} ( φ ) {e}^{- j 2 π f φ} d φ & ( 12 a ) \\ = 1 \text{/} nw {Σ}_{φ} {\underline{x}}_{0}^{T} {\underline{x}}_{φ} {e}^{- j 2 π fφ} & ( 12 b ) \\ = 1 \text{/} N {Σ}_{i} {Σ}_{w} ( {\underline{s}}_{wi}^{T} {\underline{s}}_{wi + 1} ) {e}^{- j 2 π fw} & ( 12 c )\end{matrix}$$

where S can be the power spectral density, dφ can be the phase lag w, i can be the segment number, and substitution using exemplary Eq.'s (1) and (3) can be utilized to form exemplary Eq.'s (12b) and (12c). The Fourier power spectral density calculation can decompose the autocorrelation function into its native sinusoids. Therefore, in contrast to autocorrelation spectral analysis Eq. (3), both ensemble and Fourier spectral analyses can account for periodicity at lags ensemble by averaging Eq. (9c) and Fourier by fitting sinusoids Eq. (12c).

The ensemble average of segments having length w can be a representation of correlated signal components at the corresponding frequency (e.g., =sample rate/w), and can be potentially useful for signal reconstruction. From exemplary Eqs. (7b) and (9b), an ensemble average transformation matrix can be described as, for example:

$$\begin{matrix}{T}_{w} = {U}_{w}^{T} {U}_{w} & ( 13 a ) \\ = [ \begin{matrix}{I}_{w} & {I}_{w} & … & {I}_{w} \\ {I}_{w} & {I}_{w} & … & {I}_{w} \\ & & … & \\ {I}_{w} & {I}_{w} & … & {I}_{w}\end{matrix} ] & ( 13 b )\end{matrix}$$

Signal x can then be decomposed using the linear transformation, for example:

a_{w}=1/nTw·x (14)

where a_{w} can be basis vectors, n can be as given in exemplary Eq. (5), and a_{w} and w can be N×1 in dimension. Column-wise, each identity submatrix in exemplary Eq. (13b) can serve to extract and sum one segment of w sample points in x (e.g., exemplary Eq. (14)), with the sum total being projected, for example onto the canonical basis. Row-wise the identity matrices can serve to repeat the ensemble average of length w over a total length N during construction of a w. Thus, the transformation matrix of exemplary Eq. (13) can act to decompose signals into periodic ensemble averages. Using the resulting basis vectors, signal x can be projected into ensemble space, for example:

xT·aw=1/n2wxT·Tw·x=Pw (15)

where the middle and RHS in exemplary Eq. (15) can be obtained by substitution and rearrangement using, for example, exemplary Eqs. (9), (13) and (14). Exemplary Eq. (15) states, for example, that if each signal segment of length w can be correlated with the ensemble average at w (“LHS”), the resulting correlation coefficient equals the ensemble average power (“RHS”).

In the case when N≠n·w above, the transformation matrix Tw (e.g., exemplary Eq. (13b)) can be preferably padded by N−(n·w) rows and columns, for example, by adding 0's as elements at the matrix's right edge, and adding clipped identity matrices as elements at bottom edge so the overall dimension can be N×N. Tw can be singular for all w, since two or more rows and two or more columns can typically be identical, for example, it typically has no inverse. Thus, it may generally not be possible to transform any particular basis vector a_{w} back to x, as can be intuitively obvious—an ensemble average, for example, cannot be transformed back into its original signal. Suppose now that multiple transformation equations i=1, γ can be summed, for example, for example:

$$\begin{matrix}{\underline{a}}_{w 1} + … + {\underline{a}}_{w γ} = 1 \text{/} {n}_{1} {T}_{w 1} \cdot \underline{x} + … + 1 \text{/} {n}_{γ} {T}_{w γ} \cdot \underline{x} & ( 16 a ) \\ = [ {Σ}_{i} ( 1 \text{/} {n}_{i} {T}_{wi} ) ] \cdot \underline{x} & ( 16 b )\end{matrix}$$

This can be rewritten, for example:

Σᵢa_{wi}=v= [symbol] x (17a)

[symbol]

=Σᵢ(1/nᵢT_{wi}) (17b)

where v can be the estimate of x and can be the total transform matrix. Any two basis vectors a, and aⱼ i≠j, used for construction of v, will typically be orthogonal since they can be formed from vectors in Ti versus Tj that can be orthogonal, except when i/j can be reducible to a small integer ratio. An example of a total transform matrix constructed from Ti and Tj, with dimension N=6, can be, for example:

$$𝕋 = 1 \text{/} 3 {T}_{2} + 1 \text{/} 2 {T}_{3} [ \begin{matrix}.83 & 0 & .33 & .5 & .33 & 0 \\ 0 & .83 & 0 & .33 & .5 & .33 \\ .33 & 0 & .83 & 0 & .33 & .5 \\ .5 & .33 & 0 & .83 & 0 & .33 \\ .33 & .5 & .33 & 0 & .83 & 0 \\ 0 & .33 & .5 & .33 & 0 & .83\end{matrix} ] \qquad ( 18 )$$

The magnitudes can typically be greatest along the main diagonal and equal Σ1/ni, where n can be given by exemplary Eq. (5). This matrix may not typically be invertible (e.g., Matlab ver. 7.7, R2008b). In general, as with the individual transform matrices, the total transform matrix will typically not be invertible.

Consider how T acts to transform signal x. Let a subset γ of highest basis vectors, when ranked in descending order of power, be formed from T. (See, e.g., exemplary Eq. (17)). In this case T can transfer the most correlated periodic components of the signal to form estimate v. The relative amplitude relationships of these correlated components, each extracted by a different T embedded in T, can be maintained by scale factor 1/ni during transformation (see, e.g., exemplary Eq. (16)). However, as each correlated component can typically be independent (e.g., no harmonic relationships), their combination can cause the ‘noise’ power in v to increase by √γ. To maintain the same power for best match with x, the estimate can, for example, either be scaled by 1/√γ, or alternatively, v and x can be scaled to the same power. Any unique signal structure that may not be periodic can also be transformed by T, but it can typically be via the main diagonal, for example, and not the off-diagonal elements (e.g., which sum and reinforce correlated content only). As γ can be increased, the magnitude of the main diagonal elements can increase so that T can act in part as an N×N identity matrix IN to directly transfer the unique uncorrelated detail during formation of v. So long as aᵢ and aⱼ can be approximately orthogonal, the unique detail, as well as correlated components, can maintain their correct amplitude relationships in v, since they can be added in tandem and scaled by 1/ni.

### Exemplary Atrial Electrogram Clinical Data

Exemplary clinical data was collected implementing/utilizing certain exemplary embodiments of the present disclosure. For example, atrial electrograms were recorded in a series of 20 patients, 10 with paroxysmal and 10 with longstanding persistent type, referred to the Columbia University Medical Center cardiac electrophysiology (“EP”) laboratory for catheter ablation. Two bipolar recordings of at least 10-second duration were obtained from six anatomical regions: the ostia of the left superior pulmonary veins (“LSPV”) and left inferior pulmonary veins (LIPV), the ostia of the right superior pulmonary veins (RSPV) and right inferior pulmonary veins (“RIPV”), and the anterior (“ANT”) left atrial free wall and posterior (“POS”) atrial free wall. The recordings were obtained from these regions via the distal bipolar catheter ablation electrode during sustained AF prior to any ablation. Using standard settings, the signals were filtered in hardware at acquisition to remove baseline drift and high frequency noise (e.g., first order filter pass band: 30-500 Hz). In each patient, for example, a CFAE sequence 8192 sample points long (e.g., about 0.8 seconds) as determined visually by two clinical electrophysiologists were retrospectively selected for analysis from two sites at each of the six locations. CFAE can be defined, for example, as atrial electrograms with three or more deflections on both sides of the isoelectric line, or continuous electrical activity with no well-defined isoelectric line (see, e.g., Reference 2). In all, for example, 216 of 240 recordings met these criteria, as determined by two cardiac electrophysiologists, and can be used for the exemplary further analysis. For example, no ventricular component, corresponding to the QRS deflection of the electrocardiogram, was visually evident in the CFAE. In these bipolar recordings it can be uncommon for QRS artifact to be evident in CFAE obtained from the pulmonary veins and free wall. The signals were sampled, for example, at 0.98 kHz, and stored in both raw form, and after normalization to mean zero and unity variance.

### Exemplary Tests of Fourier Versus Exemplary Ensemble Procedures

The following exemplary tests can illustrate the efficacy of the new exemplary transform versus Fourier analysis for representation of frequency and morphologic components of, for example, CFAE. The Fourier DF method can be optimized, for example, when CFAE recordings can be bipolar and approximately 8 s in length (see, e.g., References 11, 22 and 23). Accordingly, these can be used in the exemplary tests. The 8 s sequences were readily available from retrospective data, since, for example, during electroanatomic mapping, recordings with short sequence length can be commonly acquired from each site to minimize the procedure time.

### Exemplary Orthogonality of the Ensemble Basis

The inner product of ensemble basis vectors can be determined (e.g., using a computer arrangement) as, for example:

dpij=awiT·awj (19)

for all pairs i, j from w=500 to w=20 (e.g., f=2-50 Hz) for one paroxysmal and one persistent CFAE signal. The dp's can be graphed i versus j. The ensemble basis can be considered to be orthogonal if dp=1.0, i=j, and dp≈0, i≠j, except for small integer relationships in i/j. For comparison, dp can also be calculated with the Fourier basis using the same paroxysmal CFAE signal.
Exemplary Spectral Analysis of Synthetic Drivers with Phase Noise and Interference

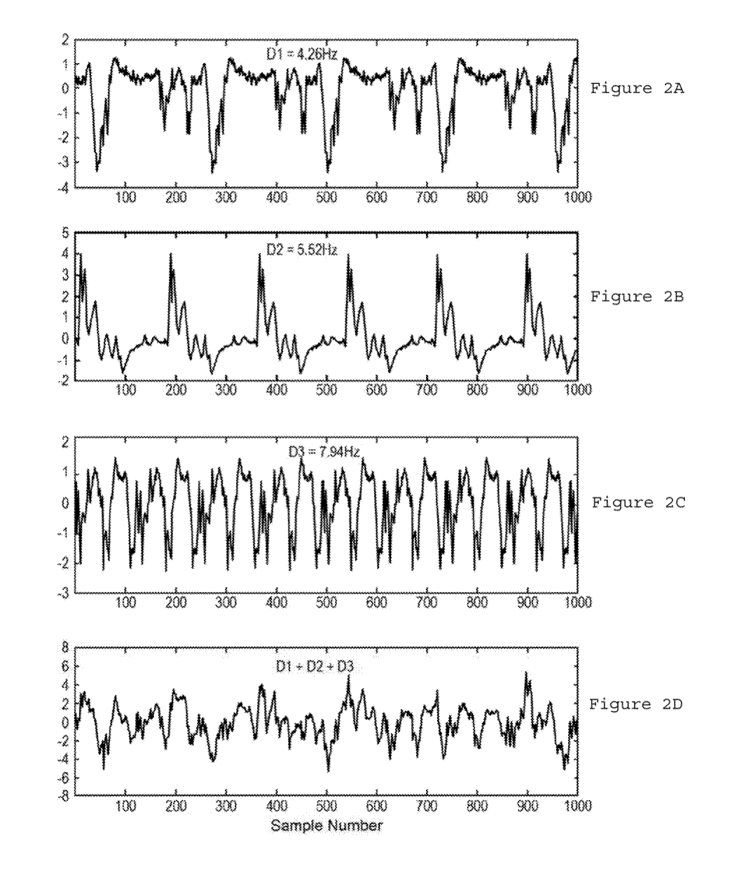

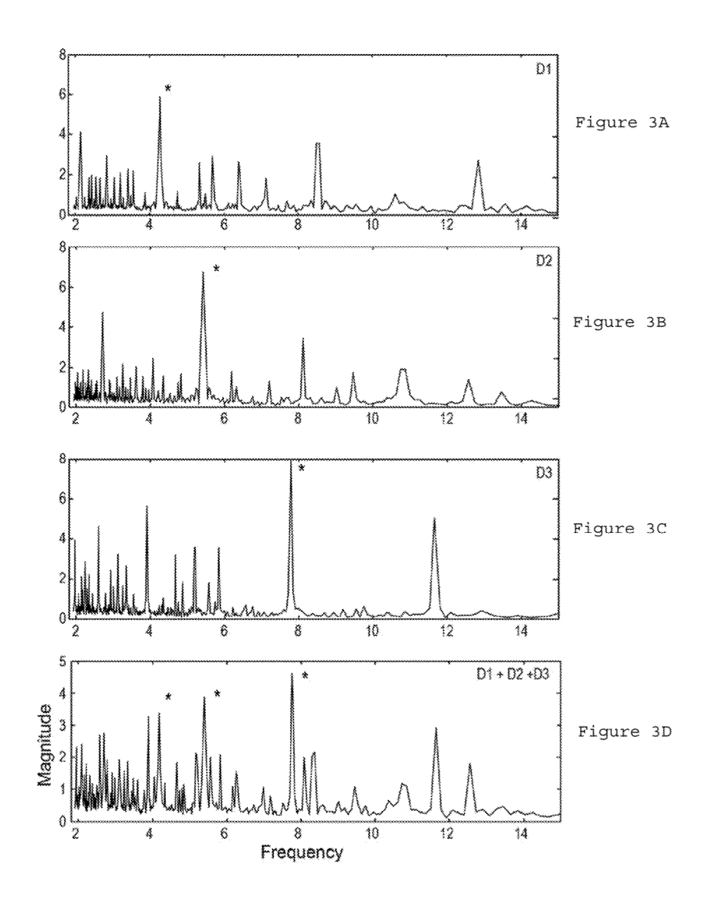

A number of, for example, three simulated independent drivers with unrelated fundamental, or DF, can be constructed, for example, from distinct CFAE deflections extracted from a single recording in one paroxysmal AF patient. The sequence lengths can be, for example, about 229 ms, about 177 ms and about 123 ms to simulate independent drivers D1, D2 and D3 with DF of about 4.37 Hz, about 5.65 Hz and about 8.13 Hz, respectively. The simulated independent generator frequencies were within the typical range of DFs that can be observed in CFAE. (See, e.g., References 2, 4 and 7). These can be normalized to mean zero and repeated to, for example, about 8192 sample points. As shown in FIGS. 2A-2D, D1 can include primarily downward deflections, D2 can include upward deflections and D3 can be biphasic. Their combination is shown, for example, in FIG. 2D. The ensemble average spectra for these simulated drivers and for their sum is shown, for example, in corresponding FIGS. 3A-3D with DFs marked by asterisks (*). The harmonics of each simulated generator may not overlap. It can also be possible that averaging segments of the ensemble average can be used to remove harmonic interaction and reduce spectral cross terms.

Phase noise can then be added by randomly and independently shifting the timing of each driver pulse (e.g., each about 229, about 177, or about 123 ms interval) using a mean zero random number generator with standard deviation of about ±16 ms. Interference can be added by summing the resulting synthetic signal D1+D2+D3 with one of about 216 scaled CFAE signals (e.g., the CFAE signals themselves acted as interference for measurement of the synthetic driver characteristics). The following combinations of gains for the phase noise random vector (p) and interference (i) can be used for assessment, for example: (p=1×, i=1×), (p=0.5×, i=2×), (p=0.3×, i=3×), and (p=0×, i=±1× . . . ±10×). Fourier and ensemble power spectra can be constructed in the range 2-10 Hz from the resulting signals. The spectral peaks can be ranked by amplitude, and the sum of ranks for peaks having frequencies of about 4.26 Hz, about 5.52 Hz, and about 7.94 Hz, with a tolerance of about ±0.2 Hz, can be tabulated. The best (e.g., minimum) sum of ranks can be, for example, 6 which can occur when the driver frequencies at about 4.26 Hz, about 5.52 Hz, and about 7.94 Hz can be ranked, for example, 1st, 2nd, and 3rd in amplitude, in certain combination, among all spectral peaks.

### Exemplary Identification of Synthetic Driver Morphology

As the exemplary ensemble procedure, aside from the Fourier transform, typically has a data-driven basis, only ensemble was used in this exemplary test. The synthetic drivers with additive phase noise and interference described in exemplary Test 2 can be corrupted using two noise gain sets, for example: p=0.3×, i=3×, and p=0×, i=5×, where the interferences i can include the 216 CFAE signals (e.g., 216 comparisons for each of the two noise gain sets). The mean squared error difference between each original synthetic driver without noise (e.g., FIGS. 2A-2D), versus the corresponding ensemble basis vector of the corrupted signal at about 123 ms, about 177 ms, and about 229 ms, the periods of the drivers, when both were normalized to unity power, can be tabulated in mV2/ms.

Exemplary Degradation of DF in CFAE with Additive Random Noise

This exemplary test can be used to determine the efficacy of each transform to detect the DF of CFAE in presence of random noise (e.g., no added synthetic drivers). For each of 20 selected CFAE with a prominent DF (e.g., sharp peak and low noise floor), random white noise can be added, for example, with a standard deviation of about 0.16 mV, approximately half that of the raw CFAE signals. The DF of the resulting CFAE signal with additive random noise can be determined. The absolute difference in DF before versus after random noise addition can be tabulated. This exemplary procedure can be repeated, for example, for 10 different additions of random noise. The mean and standard deviation in the absolute difference in DF before versus after addition of random noise can be calculated for ensemble versus Fourier spectral analysis. The entire process can be repeated for random white noise with a standard deviation of about 0.32 mV, approximately equal to the standard deviation of the raw CFAE signals.

### Exemplary CFAE Reconstruction

The exemplary 216 CFAE recordings (e.g., no added synthetic drivers) can be each decomposed and then reconstructed using 1-12 Fourier or ensemble basis vectors. The mean squared error difference between each CFAE and its reconstruction from the ordered bases can be determined. The reconstructions used can be, for example:

$$\begin{matrix}\underline{{V}_{1}} = {\underline{a}}_{w 1} {\underline{V}}_{2} \\ = {\underline{a}}_{w 1} + {\underline{a}}_{w 2} \\ {\underline{V}}_{12} = {\underline{a}}_{w 1} + {\underline{a}}_{w 2} + ⋯ + {\underline{a}}_{w 12}\end{matrix} \qquad ( 20 )$$

where a w1 to a w12 can be, for example, the top 12 basis vectors in descending order of power. The average error can be determined for Fourier versus ensemble reconstruction.

### Exemplary Single Driver Test

The CFAE signals can then be altered or modified by adding a low-power transient component at, for example, about 200 sample point intervals (e.g., about 977 samples per second/200 samples ˜5 Hz). The transient itself can include a 42 sample point long biphasic component extracted from a CFAE acquired from the LSPV ostia during persistent AF. This transient can have properties of mean=0.13 mV, standard deviation=0.54 mV, and peak-peak values of ˜about ±1 mV. CFAEs after addition of the low-power transient can be analyzed using ensemble and Fourier spectral analysis to determine whether the component can be readily identified. Identification can be defined to be presence of a distinct power spectral peak, with the base of the peak reaching the surrounding noise floor.

For the exemplary tests described above, the ensemble averaging power spectrum was generated as described by exemplary Eqs. (9)-(11) and the accompanying text. Exemplary Fortran code used for ensemble spectra calculation can be provided in the Appendix and it can be written, for example, to approximately halve the computation time by calculating:

ew/2(1:w/2)=ew(1:w/2)+ew(w/2+1:w) (21)

The Fourier power spectrum can be computed/determined (e.g., with the computer arrangement) using MATLAB (e.g., ver. 5.1, 1997, Mathworks) by applying, for example, a Hanning window to the exemplary 8192 discrete point signal. Note that to prevent signal distortion, the traditional Fourier preprocessing method of bandpass filtering, rectification and low pass filtering may not be used. A Fast Fourier Transform (“FFT”) can be then computed from the windowed signal and the power spectrum can be graphed. The t-test and f-test can be used for statistical comparison of means and variances, with significance considered to be, for example, p<0.05 (e.g., SigmaPlot ver. 9.0, Systat Software, 2004, and MedCalc ver. 9.5, MedCalc Statistical Software 2008).

### Exemplary Improved Frequency Resolution for Characterization of CFAE

Atrial electrograms were recorded in a series of 20 patients referred to the Columbia University Medical Center cardiac EP laboratory for catheter ablation of AF. Ten patients had documented clinical paroxysmal AF, and all 10 had normal sinus rhythm as their baseline rhythm in the EP laboratory. AF was induced by burst atrial pacing from the coronary sinus or right atrial lateral wall, and persisted for at least 10 minutes for those signals included in the retrospective analysis of this study. Ten other patients had longstanding persistent AF, and had been in AF without interruption for 1-3 years prior to the catheter mapping and ablation procedure. The surface electro gram signals were acquired in analog form using the GE CardioLab system (e.g., GE Healthcare, Waukesha, Wis.) and filtered from about 30-500 Hz with a single-pole band pass filter to remove baseline drift and high frequency noise. The filtered signals were digitally sampled by the system at about 0.977 KHz and stored. Although the band pass high end was slightly above the Nyquist frequency, negligible signal energy can be expected to reside in this frequency range.

Only signals identified as CFAEs by two cardiac electro physiologists were included in the retrospective analysis. Candidate CFAE recordings of at least 10 seconds in duration were obtained from two sites outside the ostia of each of the four pulmonary veins (“PV”). Similar recordings were obtained at two sites on the endocardial surface of the left atrial free wall, one in the mid-posterior wall, and another on the anterior ridge at the base of the left atrial appendage.

From each of these recordings, about 8.4-second sequences (e.g., about 8192 sample points) were analyzed. A total of 240 such sequences were acquired during electrophysiologic analysis—120 from paroxysmal and 120 from longstanding AF patients. Subsequently, only about 216 of the recordings were determined to be CFAE, and only these were used for subsequent analysis. As in previous studies, all CFAE signals were normalized to mean zero and unity variance prior to further processing.

### Exemplary Identification of Recurring Patterns in Fractionated Atrial Electrograms

Exemplary electrograms were recorded in a series of twenty patients referred to the Columbia University Medical Center cardiac EP laboratory for catheter ablation of AF. Ten patients had documented clinical paroxysmal (e.g., acute) AF, with a normal sinus rhythm as their baseline rhythm in the electrophysiology laboratory. Atrial fibrillation was induced by burst pacing from the coronary sinus or the lateral right atrial wall, and the arrhythmia persisted for at least 10 minutes for those signals to be included in the retrospective analysis. Ten other patients had persistent (e.g., longstanding) AF, and had been in AF without interruption for 1-6 years prior to the catheter mapping. Only digitized signals identified as CFAE by two cardiac electrophysiologists were included in the retrospective analysis. The CFAE recordings were obtained from two sites outside the ostia of each of the four PVs. Similar recordings were obtained at two sites on the endocardial surface of the left atrial free wall, one in the mid-posterior wall, and another on the anterior ridge at the base of the left atrial appendage. From each of these recordings, about 8.4-second sequences (e.g., about 8192 sample points) were extracted and analyzed. A total of about 240 such sequences were acquired—120 from paroxysmal and 120 from longstanding AF patients. Subsequently, only about 216 of the recordings were confirmed as CFAE, and only these were used for subsequent analysis. All CFAE signals were normalized to mean zero and unity variance prior to further processing and ablation procedure. Bipolar electrograms of at least 10 seconds in duration, recorded from the distal ablation electrode during arrhythmia, were bandpass filtered by the system at acquisition to remove baseline drift and high frequency noise (e.g., about 30-500 Hz), sampled at 977 Hz and stored. Although the bandpass high corner was slightly greater than the Nyquist frequency, negligible signal energy resides in the region.

Only digitized signals identified as CFAE by two cardiac electrophysiologists were included in the retrospective analysis. The CFAE recordings were obtained from two sites outside the ostia of each of the four PVs. Similar recordings were obtained at two sites on the endocardial surface of the left atrial free wall, one in the mid-posterior wall, and another on the anterior ridge at the base of the left atrial appendage. From each of these recordings, about 8.4-second sequences (e.g., about 8192 sample points) were extracted and analyzed. A total of about 240 such sequences were acquired—120 from paroxysmal and 120 from longstanding AF patients. Subsequently, only about 216 of the recordings were confirmed as CFAE, and only these were used for subsequent analysis. All CFAE signals were normalized to mean zero and unity variance prior to further processing.

### Exemplary Spectral Profiles of Complex Fractionated Atrial Electrograms

Atrial electrograms were recorded in a series of 20 patients referred to the Columbia University Medical Center cardiac EP laboratory for catheter ablation of AF. Ten patients had documented clinical paroxysmal AF, and all 10 had normal sinus rhythm as their baseline cardiac rhythm in the cardiac electrophysiology laboratory. AF was induced acutely by burst atrial pacing from the coronary sinus or right atrial lateral wall, and allowed to persist for at least 10 minutes prior to data collection. Patients in whom only short runs of AF were inducible were excluded from this study. Ten other patients had longstanding persistent AF, and had been in AF without interruption for 6 months to 6 years prior to their catheter mapping and ablation procedure.

The duration of uninterrupted AF in these patients was estimated as the period from the time of recurrence of AF after the last DC cardioversion (e.g., which converted AF to sinus rhythm) to the day of the catheter ablation procedure. Bipolar atrial mapping was performed with a NaviStar ThermoCool catheter, 7.5 F, about a 3.5 mm tip, with about a 2 mm spacing between bipoles (e.g., Biosense-Webster Inc., Diamond Bar, Calif., USA). The electrogram signals were acquired using the GE CardioLabsystem (e.g., GE Healthcare, Waukesha, Wis., USA), and filtered at acquisition from about 30 to about 500 Hz with a single-pole bandpass filter to remove baseline drift and high frequency noise. The filtered signals were digitally sampled by the system at about 0.977 KHz and stored. Although the bandpass high end was slightly above the Nyquist frequency, negligible CFAE signal energy resides in this frequency range 10. Only signals identified as CFAEs by 2 cardiac electrophysiologists were included in this analysis. 9, 10 and 12 CFAE recordings of at least 10 seconds in duration were obtained from 2 sites outside the ostia of each of the 4 PVs. Similar recordings were obtained at 2 left ANT free wall (“FW”) sites, one in the mid POS wall, and another on the anterior ridge at the base of the left ANT appendage. The mapping catheter was navigated in these prespecified areas until a CFAE site was identified. In 1 patient with clinical paroxysmal AF, during acutely induced AF, no recording site outside the PVs with recordings satisfying CFAE criteria for at least 10 seconds could be detected. Therefore, data from this patient were not included in the following analysis. From each of the exemplary recordings described above, when a CFAE sequence over about 16.8 s was recorded during AF, 2 consecutive about 8.4 s series were extracted and analyzed. Only sites at which the CFAE criteria were maintained during the recorded sequence were used for analysis. A total of about 204 sequences 90 from paroxysmal and about 114 from longstanding AF patients, all meeting the criteria for CFAE—were chosen for this study and included in the following analysis. As in the previous studies, to standardize the morphological characteristics, all CFAE signals were normalized to mean zero and unity variance (e.g., average level=0 volts, standard deviation=1).

To remove the second harmonic, which can usually be the predominant sub- or super-harmonic, an exemplary antisymmetry technique was applied to each ensemble average.

### Exemplary Results Exemplary Orthogonality of the Ensemble Basis

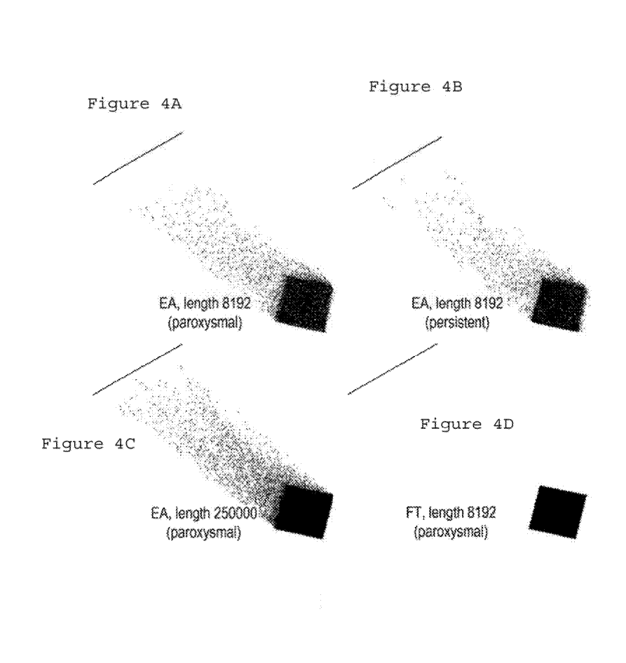

An exemplary result of the inner product measurement (exemplary Eq. (19)) can be shown, for example, in the graphs of FIGS. 4A-4D, which can be generated using, for example, map 3D, an interactive scientific visualization tool for bioengineering data devised by the Scientific Computing and Imaging Institute, University of Utah. (See, e.g., Reference 24). As shown in each figure, the DP magnitude scale can increase, for example, from 0 to 1 from lower right to upper left. As shown in FIGS. 4A-4C, the exemplary result for the exemplary ensemble method can be shown, computed for all bases a500-a20 (e.g., 481 basis vectors ranging from 2 Hz-50 Hz). As shown in FIG. 4A (e.g., paroxysmal AF) DP values, for example, can be near zero when i≠j, (e.g., fuzzy square region). A line can be formed at unity magnitude at upper left, corresponding to i=j (e.g., autocorrelation). Where i and j can be harmonically related, the DP magnitude can be intermediate (e.g., few scattered points between lower right and upper left).

A similar result can be obtained for the persistent AF signal (B). For all values i≠j including those that were harmonically related, the mean normalized inner product can be, for example, about 0.0075±0.0510 for 108 paroxysmal CFAE and about 0.0077±0.0509 for 108 persistent CFAE signals (e.g., <1% of the magnitude when i=j). For N=8192, random cancellation of uncorrelated components may have been incomplete. As a further test, the basis vectors for the paroxysmal CFAE signal can be extended, for example, to N=250,000 in length, and the resulting inner products can be provided in the exemplary illustration of FIG. 4C. In the exemplary illustration of FIG. 4C, when i≠j and no harmonic relationship exits, dp=0.0 (e.g., square region can be solid rather than fuzzy, for example, there can be complete cancellation of random components). Thus, the exemplary ensemble basis can be orthogonal except for small integer harmonic relationships. For comparison, the dp using Fourier bases (e.g., N=8192) can be shown in the exemplary illustration of FIG. 4D. Since the sinusoidal basis can be antisymmetric about the x-axis, the inner product can be zero when i≠j, even for harmonic relationships.

Exemplary Spectral Analysis of Synthetic Drivers with Phase Noise and Interference

FIGS. 5A and 5B illustrate, for example, exemplary graphs of Fourier and ensemble spectra of the three synthetic drivers when interference can be added (e.g., p=0×, i=5×). Most of the spectral components can typically be caused by the drivers, with the interference contributing to the noise floor (e.g., compare the exemplary graphs of FIGS. 5B and 3D from 2-10 Hz). The location of synthetic driver peaks is noted in FIGS. 5A and 5B by asterisks (*). Portions of the noise floor can extend beyond two driver peaks in the Fourier spectrum (see FIG. 5A). The driver peaks can be all higher than the noise floor in the ensemble spectrum (see FIG. 5B). There can be more detail at the lower end of the ensemble spectrum due to the w=1/f relationship (exemplary Eq. (6)), and sub-harmonics can also be evident. The exemplary result for measurements with the various additive noise combinations and interferences is shown, for example, in Table 1.

TABLE 1

| P | 1 | Fourier | Ensemble | MN | SD |
|---|---|---|---|---|---|
| 1x | 1x | 7.12 ± 1.41 | 6.71 ± 1.08 | .005 | NS |
| .5x | 2x | 7.03 ± 0.48 | 6.31 ± 0.10 | <.001 | <.001 |
| .3x | 3x | 7.88 ± 0.30 | 6.73 ± 0.08 | <.001 | <.001 |
| 0x | ±10x | 10.24 ± 3.37 | 8.82 ± 3.08 | <.001 | NS |

p = gain of added phase noise
i = gain of added interference.
MN, SD = significance of mean and standard deviation.

In the first and second columns of Table 1, the phase and interference multipliers, respectively, are shown. In the third and fourth columns, mean±standard deviation in the sum of ranks for D1, D2, and D3 are shown. The significance of the differences is noted in the last two columns. All of the means can be significantly different, with the synthetic drivers, for example, being more highly ranked in the ensemble spectra (e.g., total rank can be closer to 6). The standard deviation in total rank, for example, the variability, can be higher in Fourier as compared with ensemble, with a significant difference in two cases.

### Exemplary Identification of Synthetic Driver Morphology

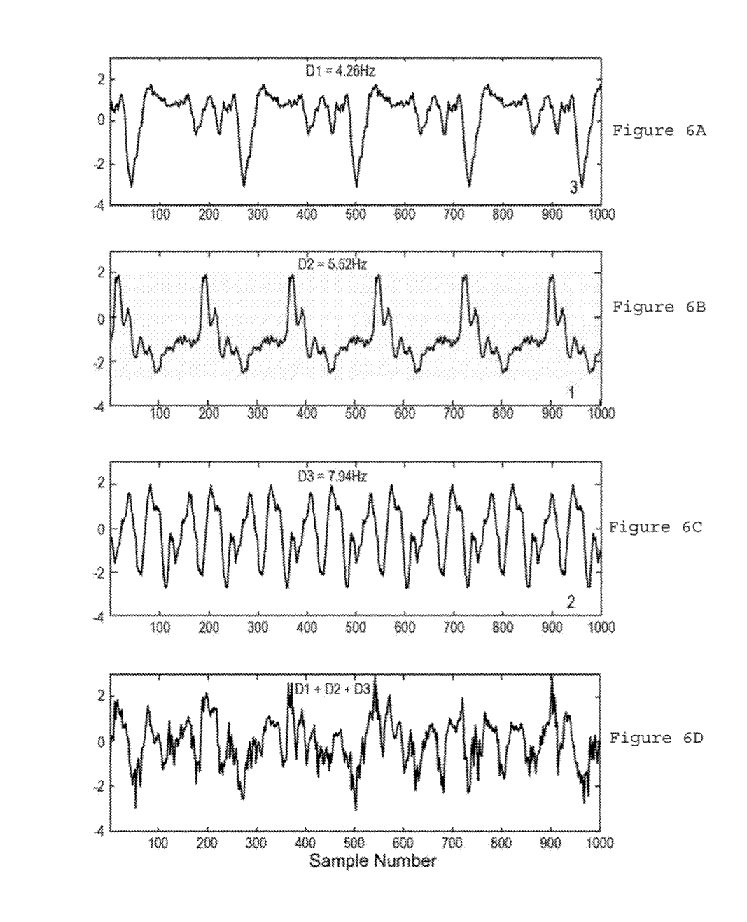

FIGS. 6A-6D show exemplary graphs for the top three basis vectors constructed from synthetic drivers with noise and interference added (e.g., weighting p=0.3×, i=3×). The basis vectors shown in FIGS. 6A-6C can be reflective of the corresponding original drivers depicted in FIG. 2. Some smoothing can occur in the fine detail due to the phase noise (e.g., jitter) that can be added to the drivers. The about 4.37 Hz, about 5.65 Hz and about 8.13 Hz bases can be ranked the 3rd, 1st, and 2nd highest peaks, respectively, in the ensemble power spectrum, as is noted at bottom right in each of FIGS. 6A-6C. For the noise set (e.g., p=0×, i=5×) the corresponding basis vectors can estimate the FIG. 2 drivers, as there was no added jitter. For the exemplary 216 tests with phase noise and interference (e.g., p=0.3×, i=3×) the average mean squared error can be about 0.091±0.020 mV2/ms, while for additive interference only (e.g., p=0×, =5×) it can be only about 0.0049±0.0042 mV2/ms. These errors can be, for example, <10% of the power in the normalized drivers (e.g., 1.0 mV2/ms). Thus, in the presence of jitter and/or interference, the morphology of independent drivers in CFAE can be extractable using the ensemble basis.

Exemplary Degradation DF in CFAE with Additive Random Noise

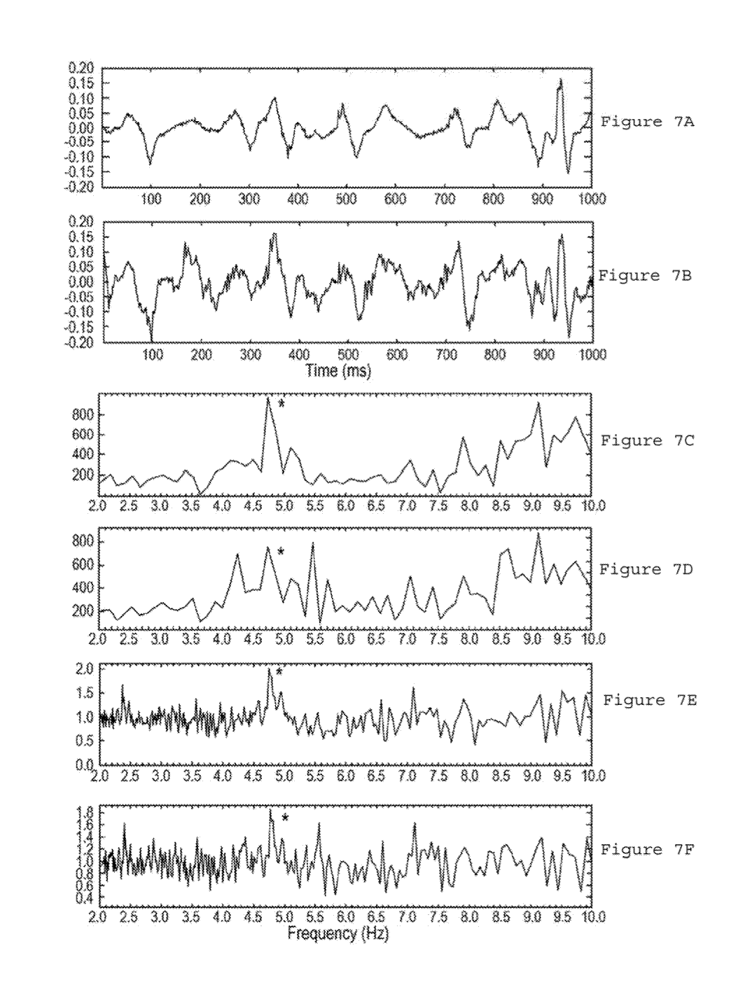

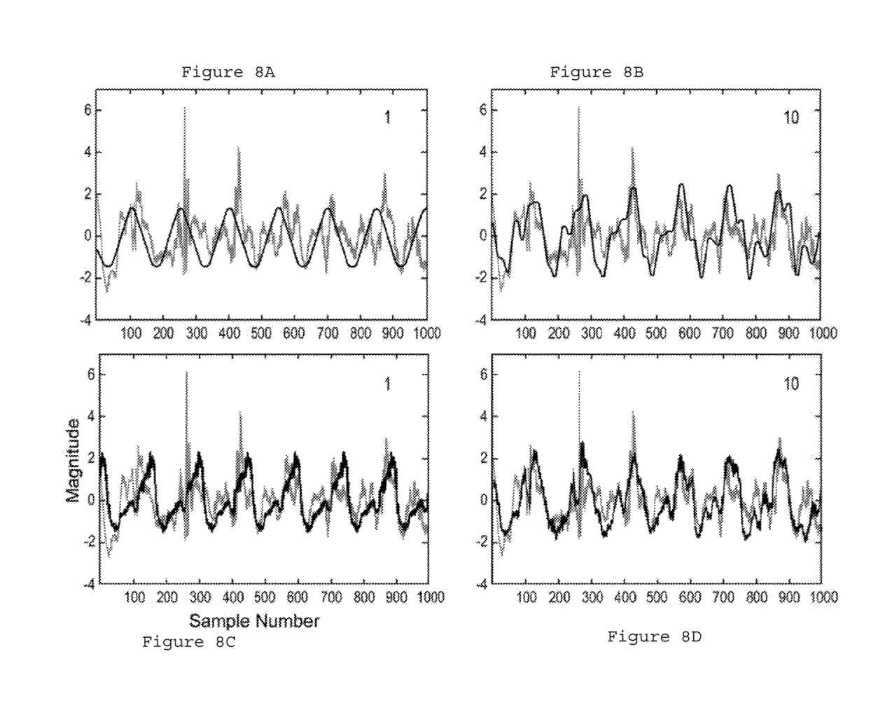

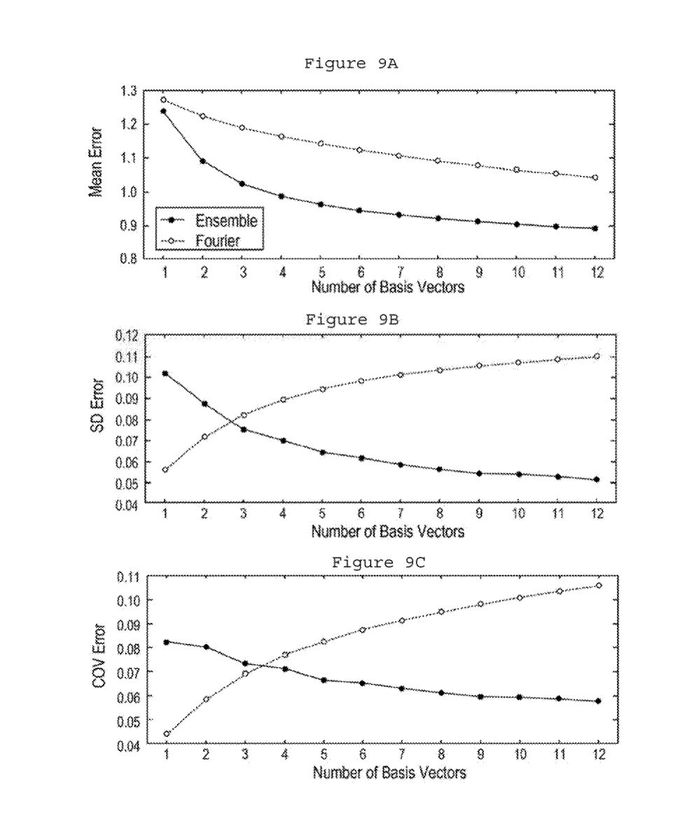

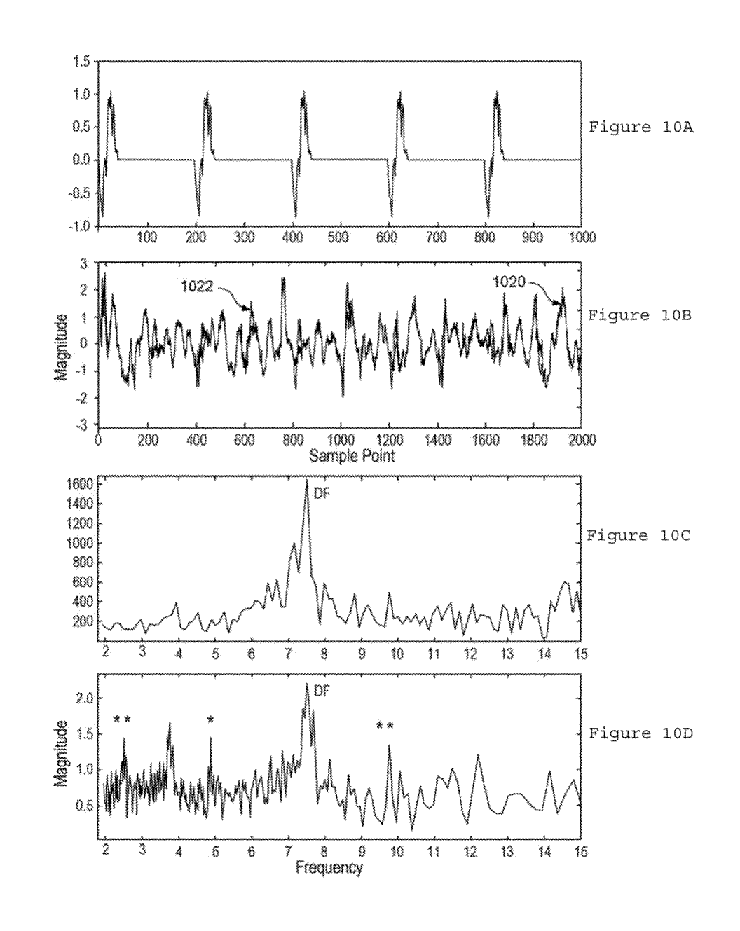

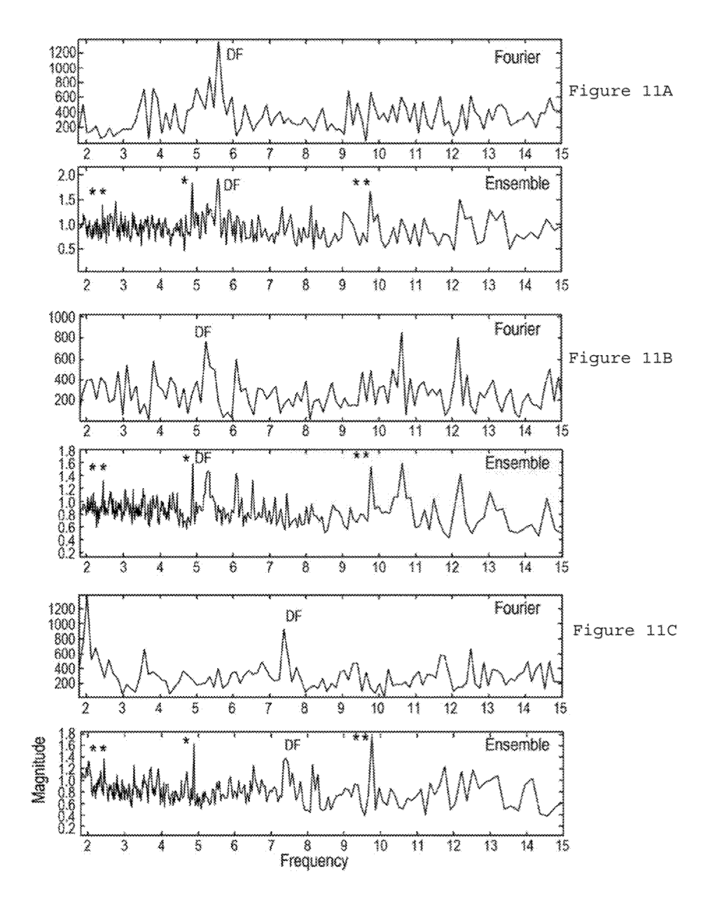

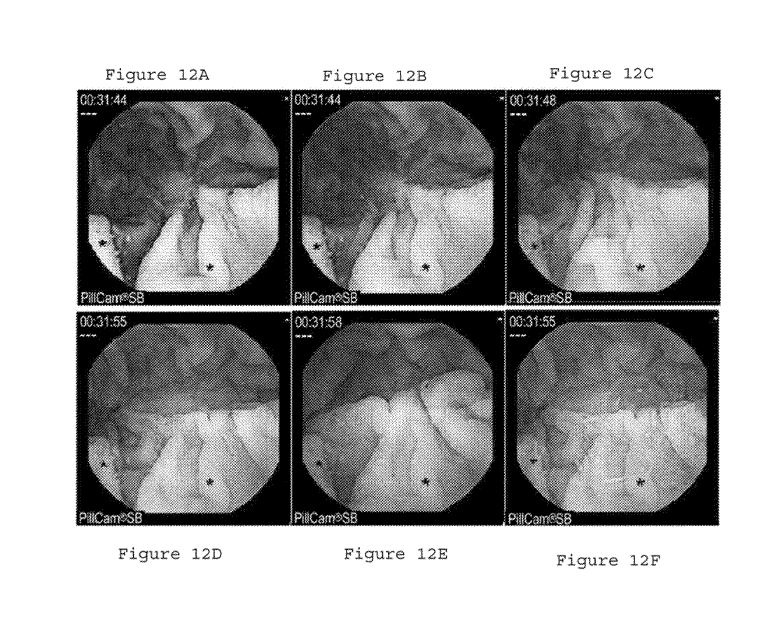

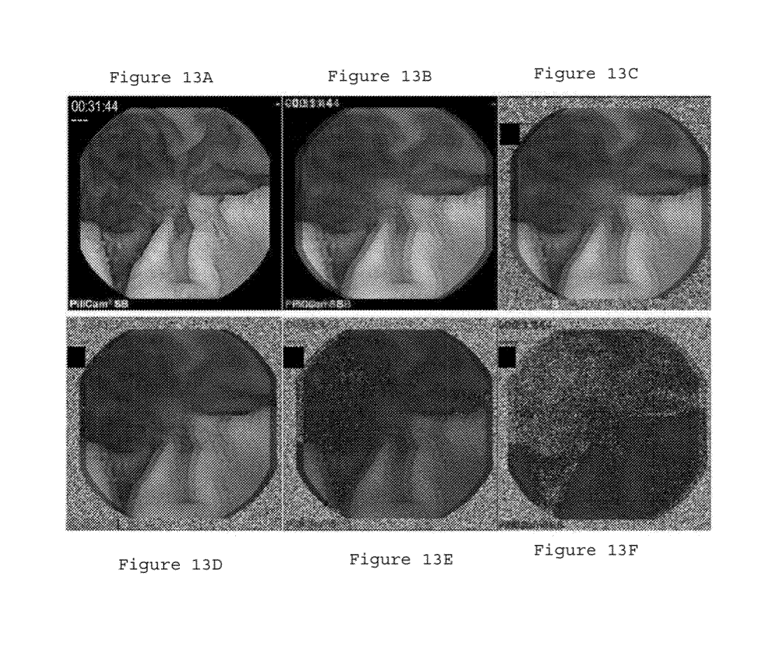

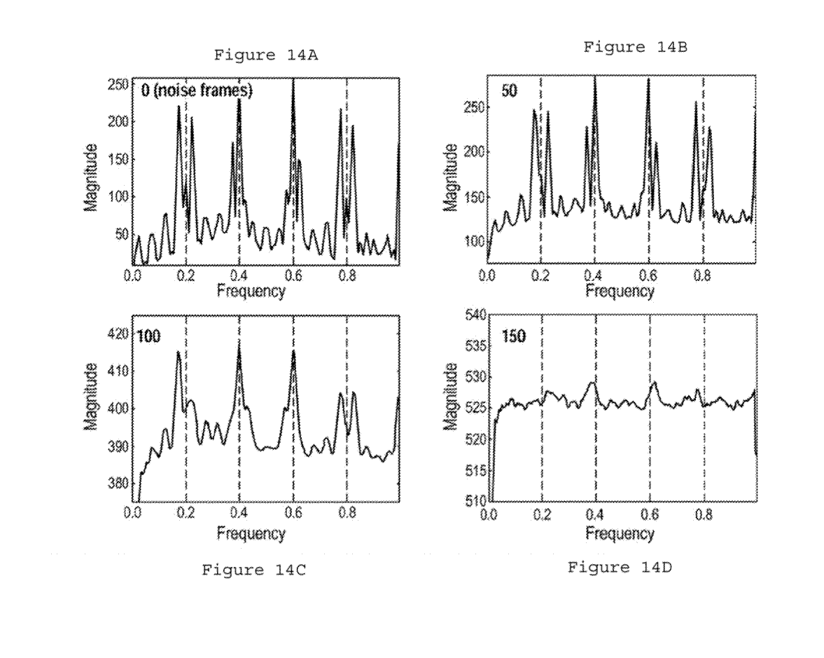

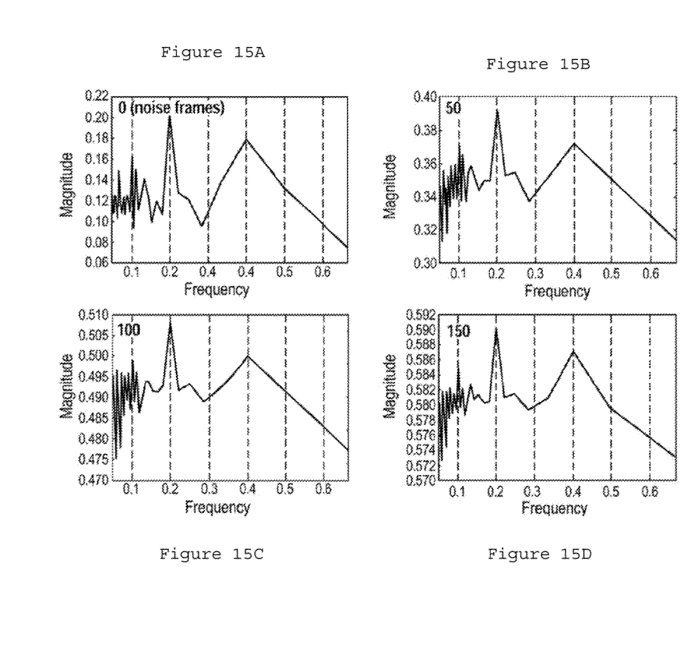

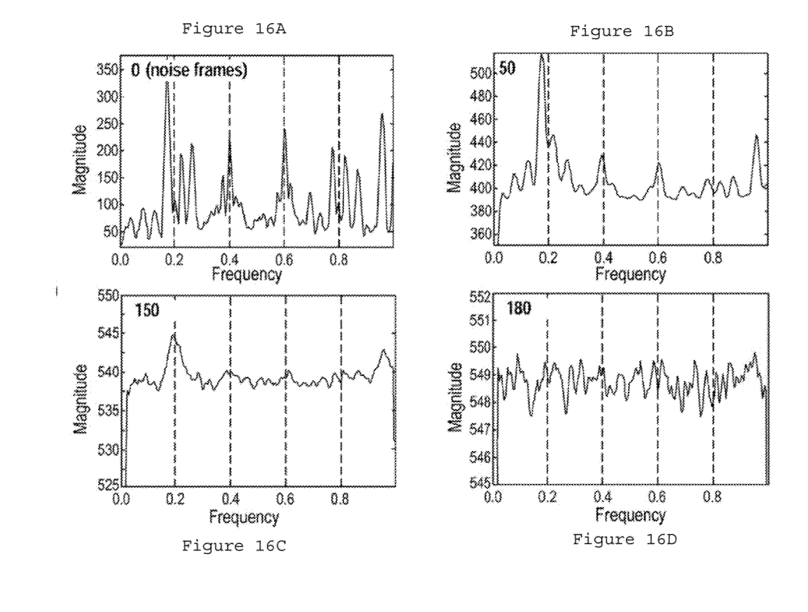

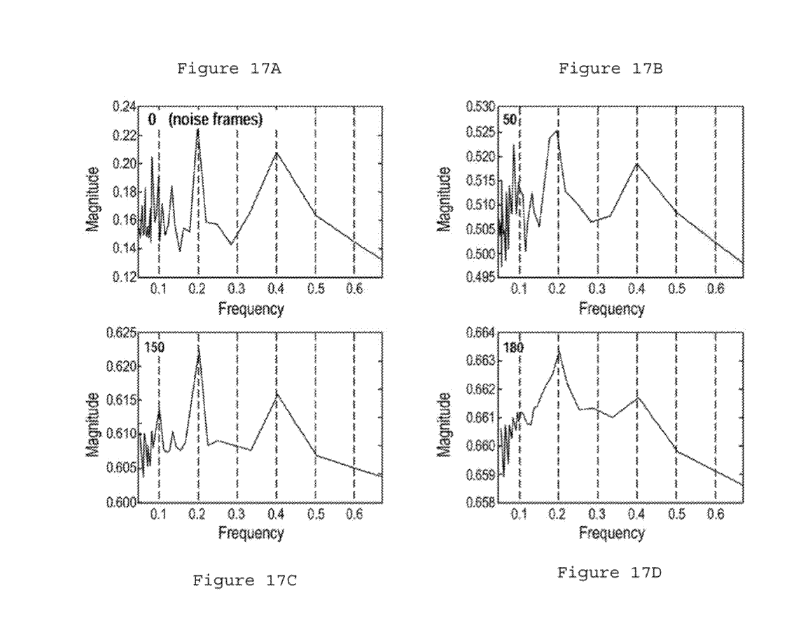

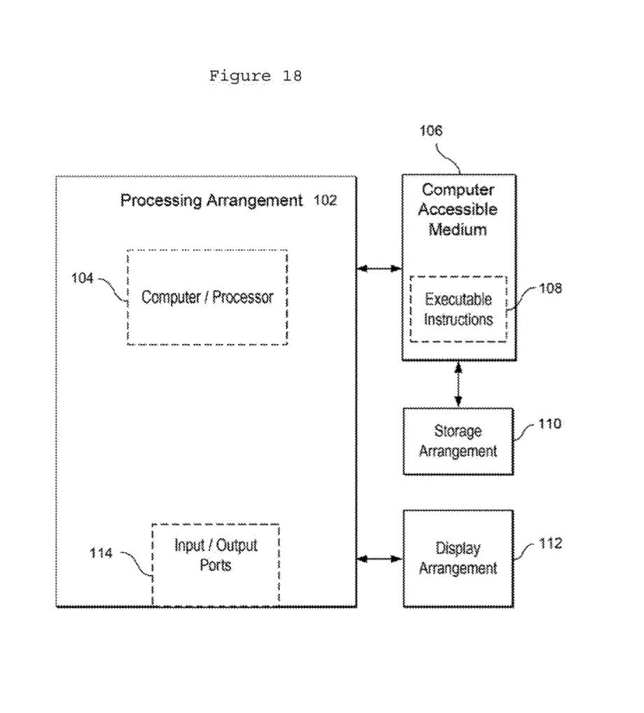

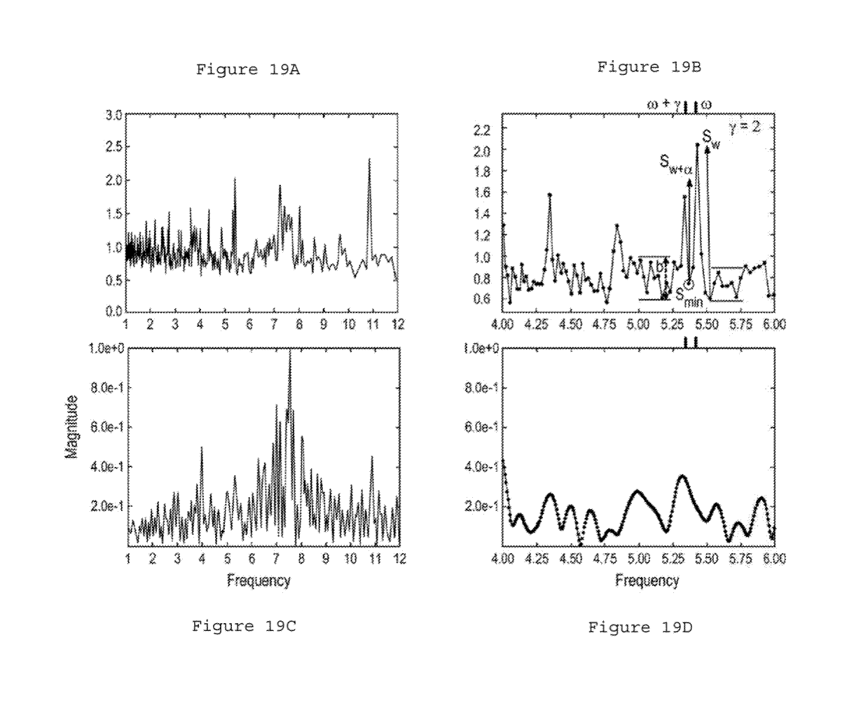

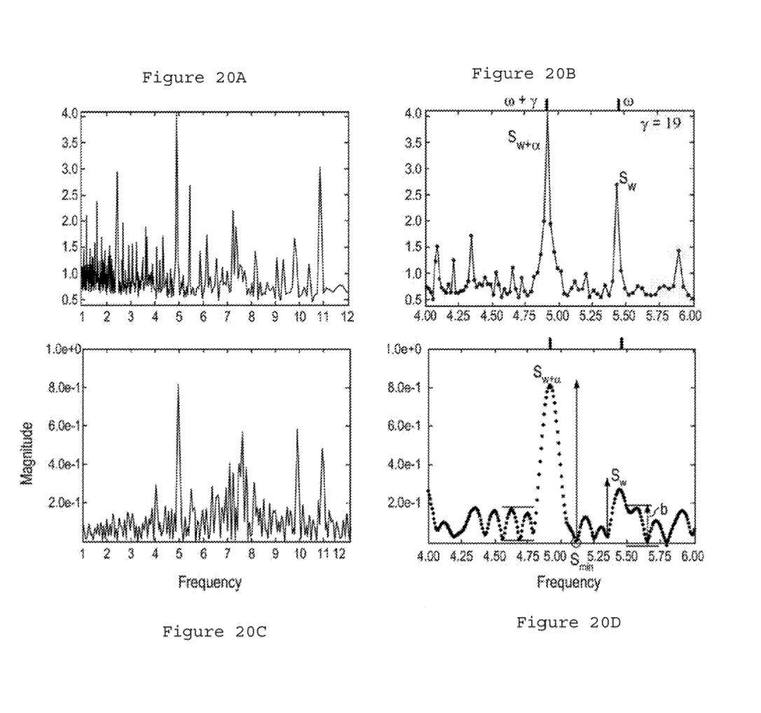

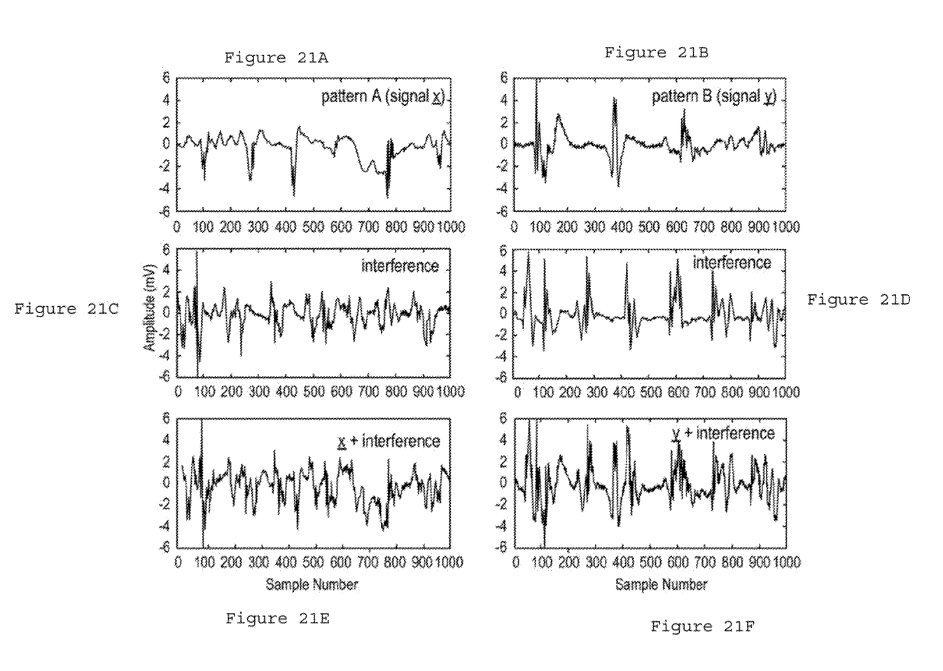

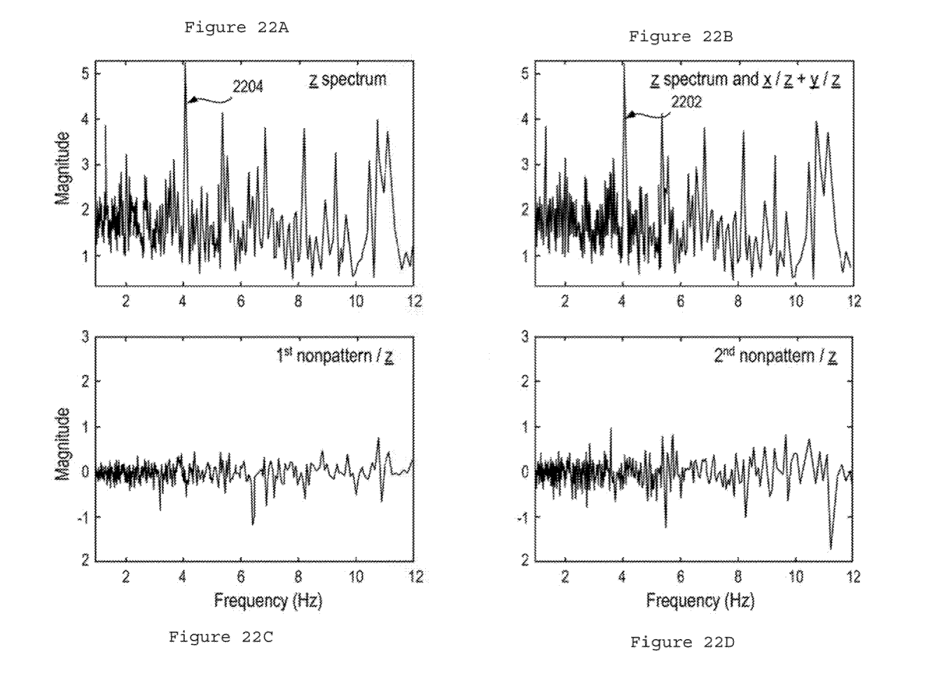

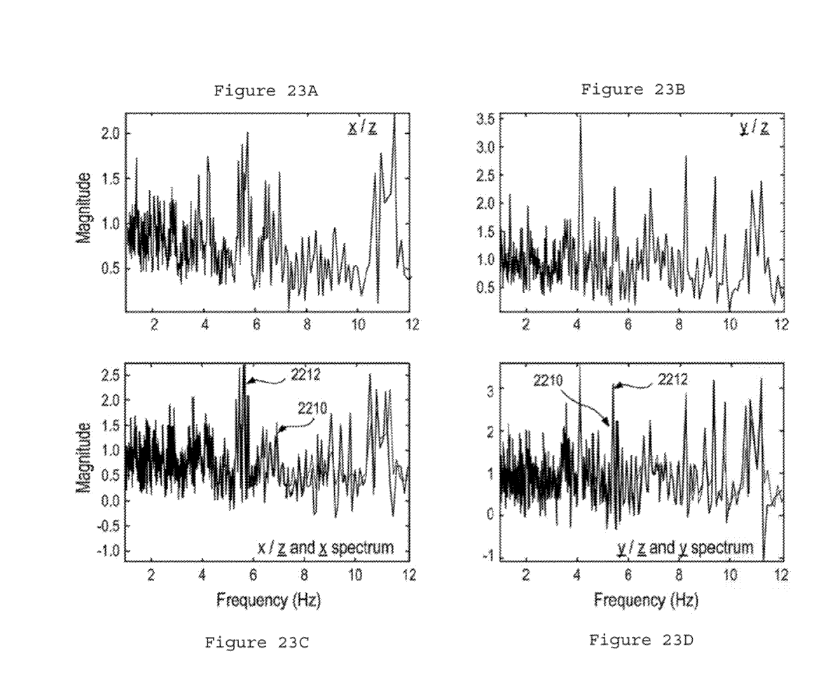

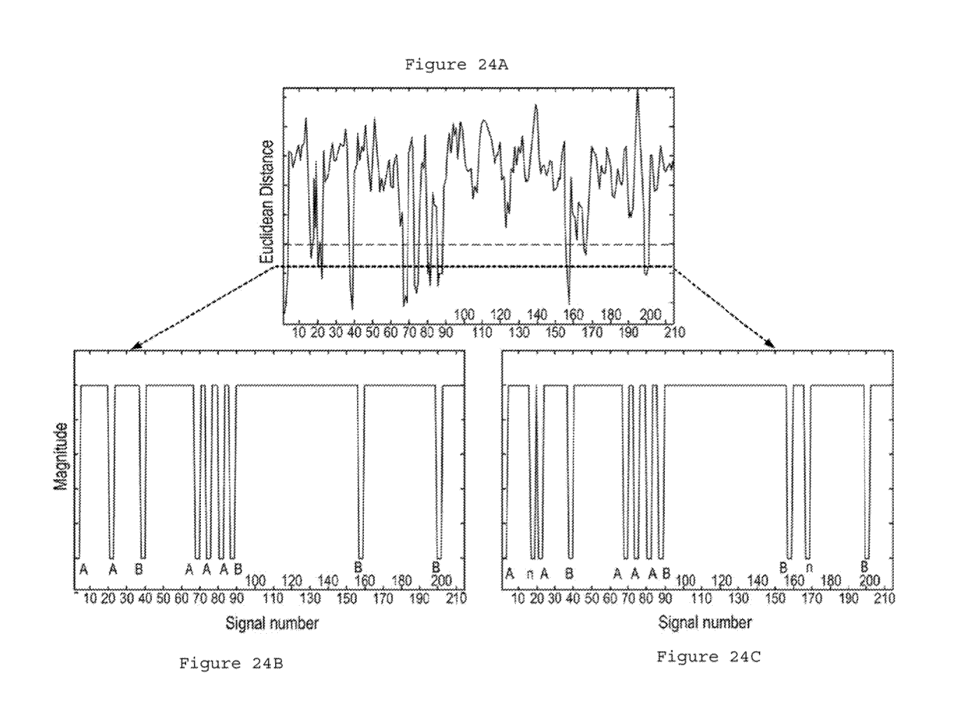

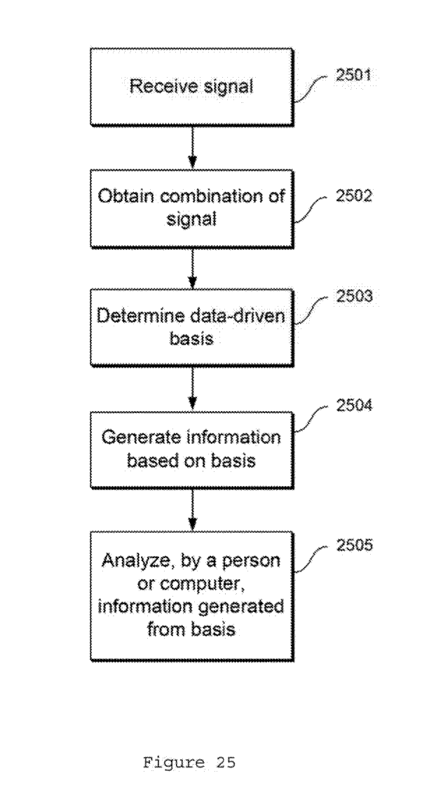

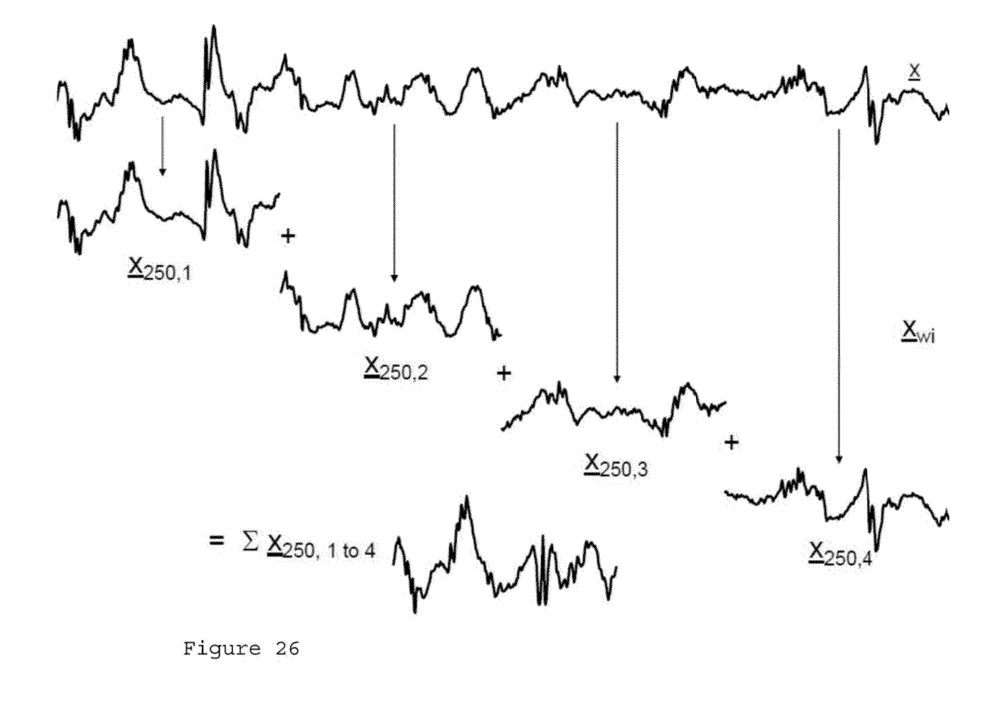

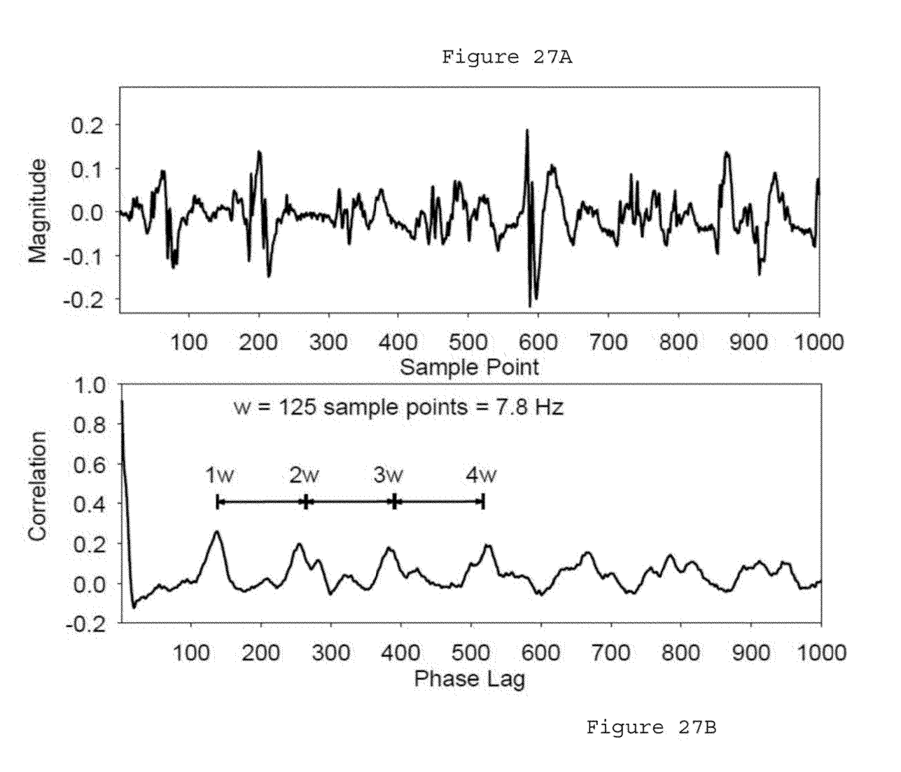

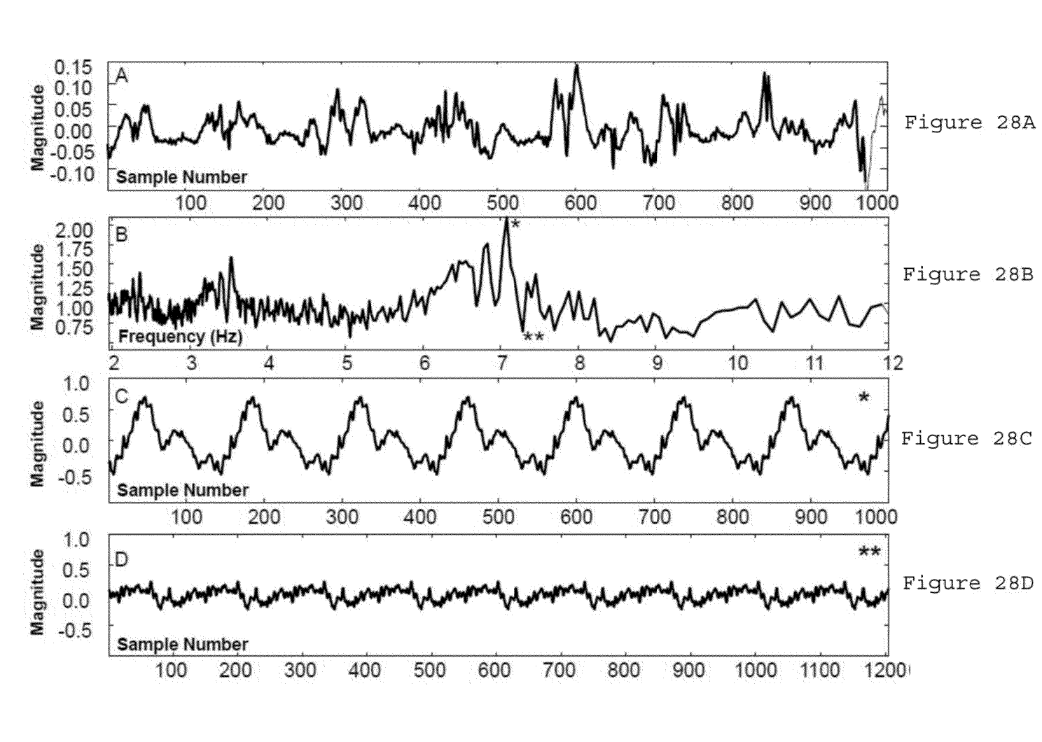

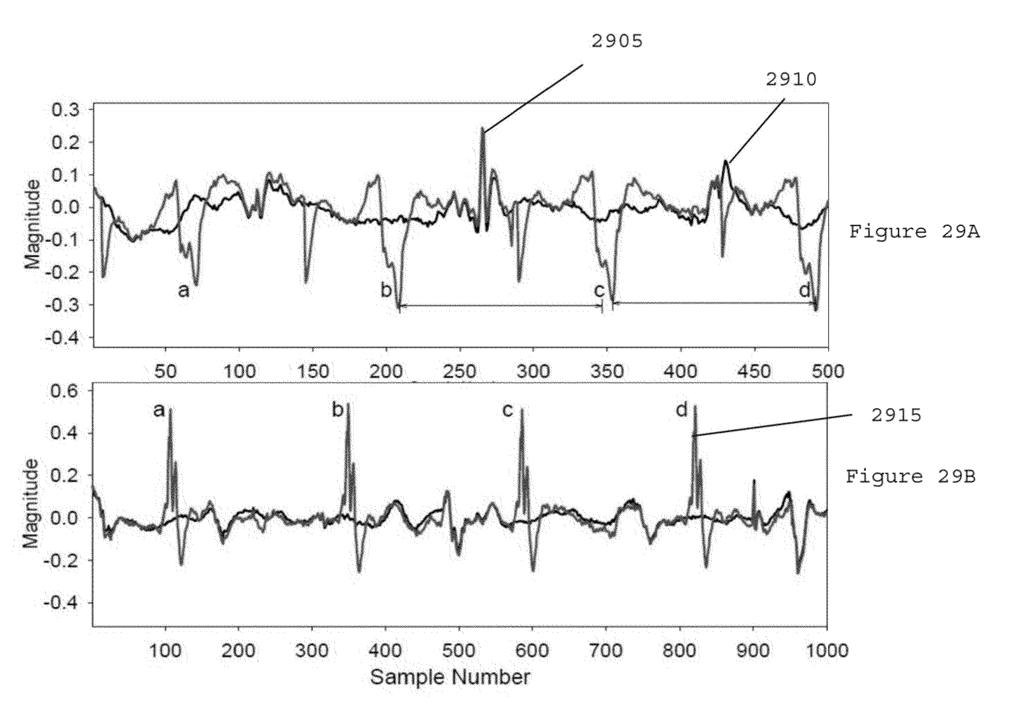

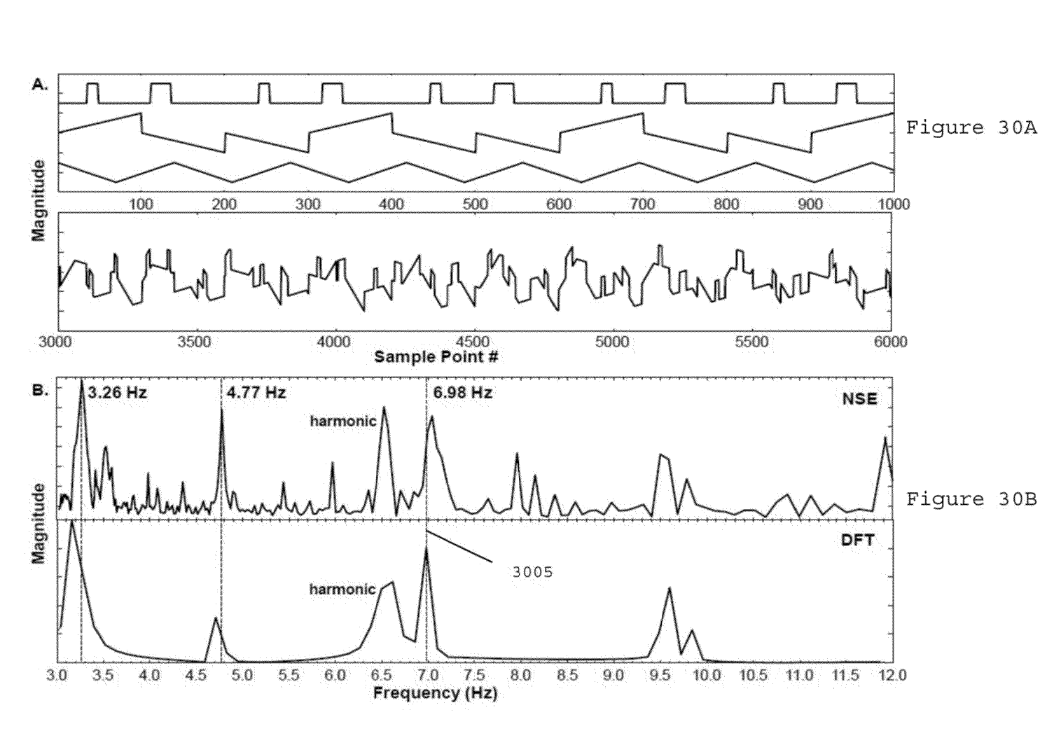

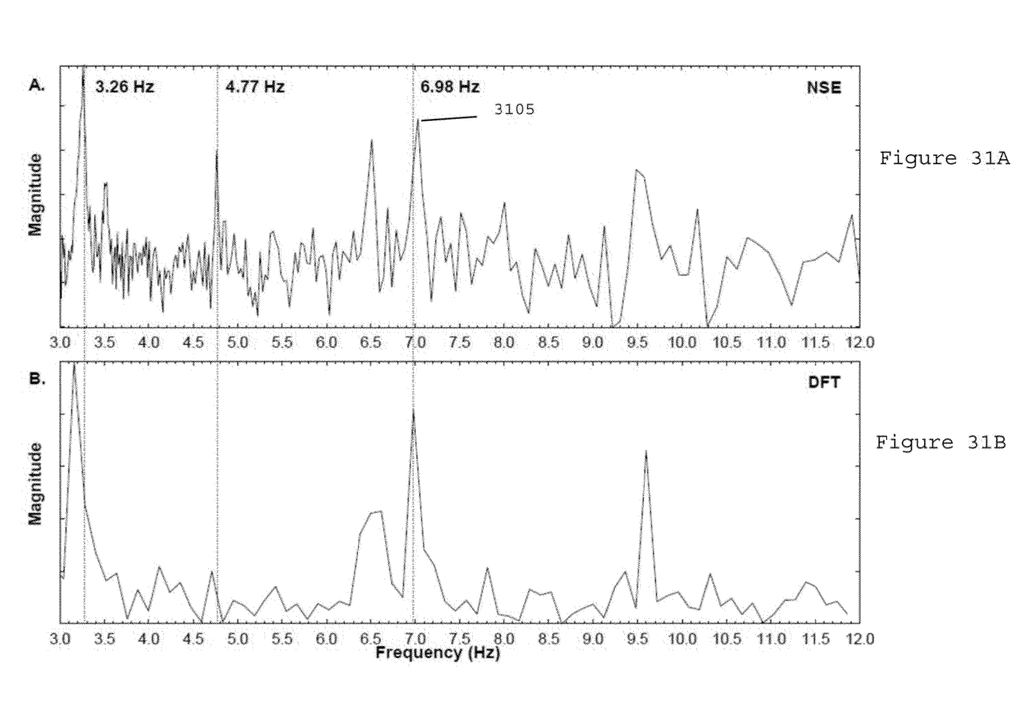

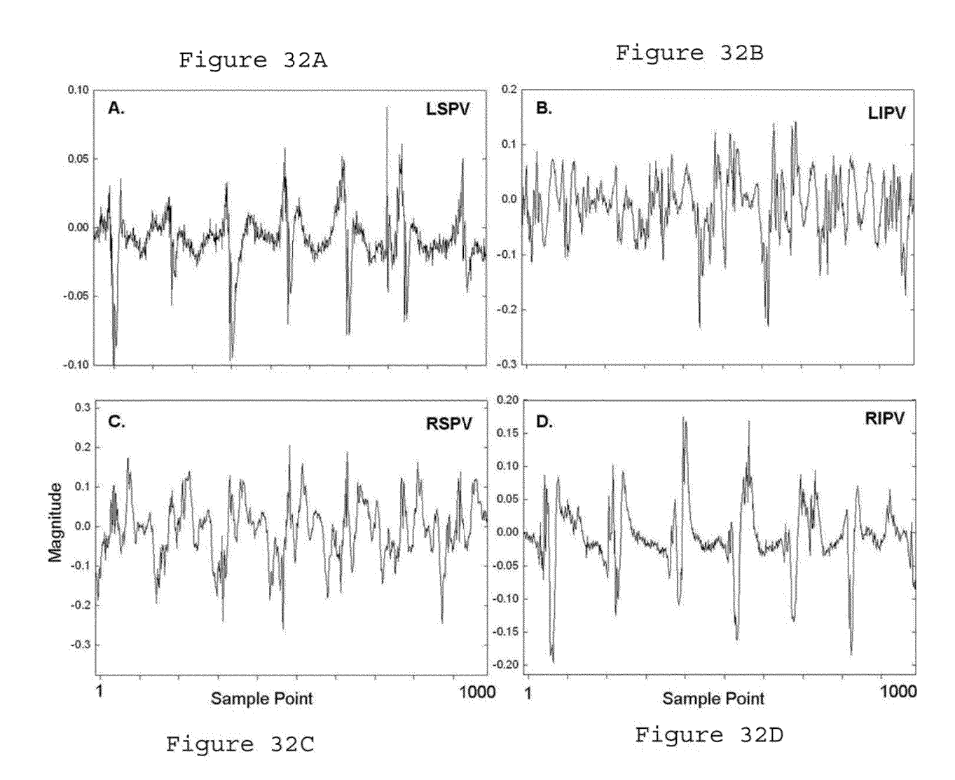

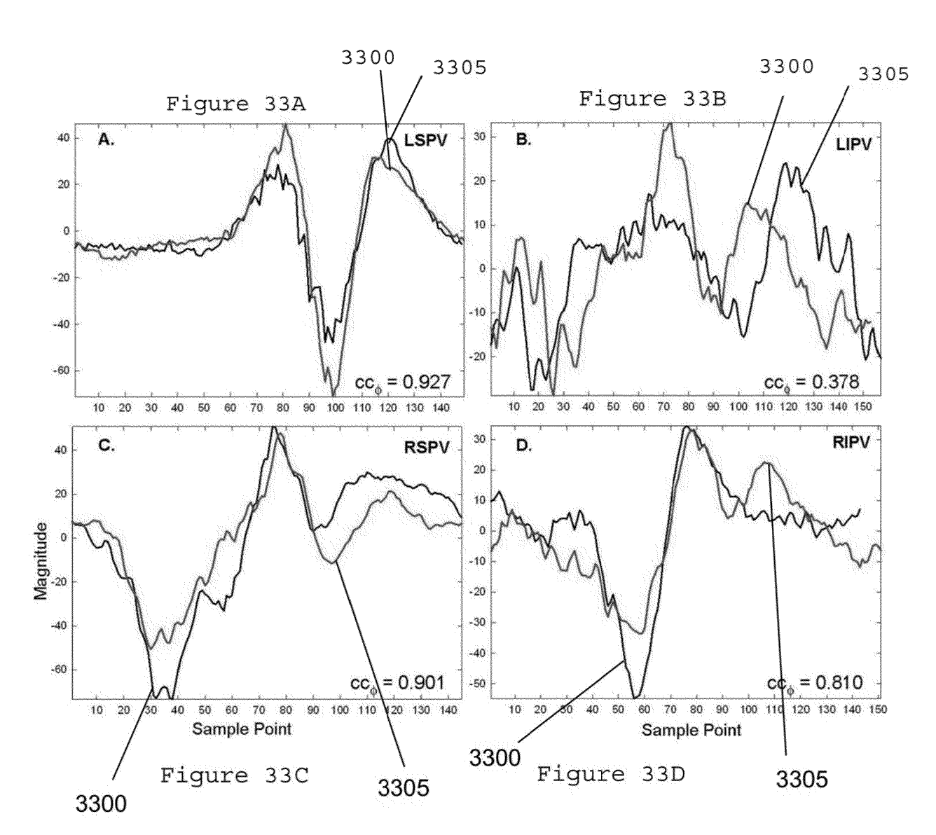

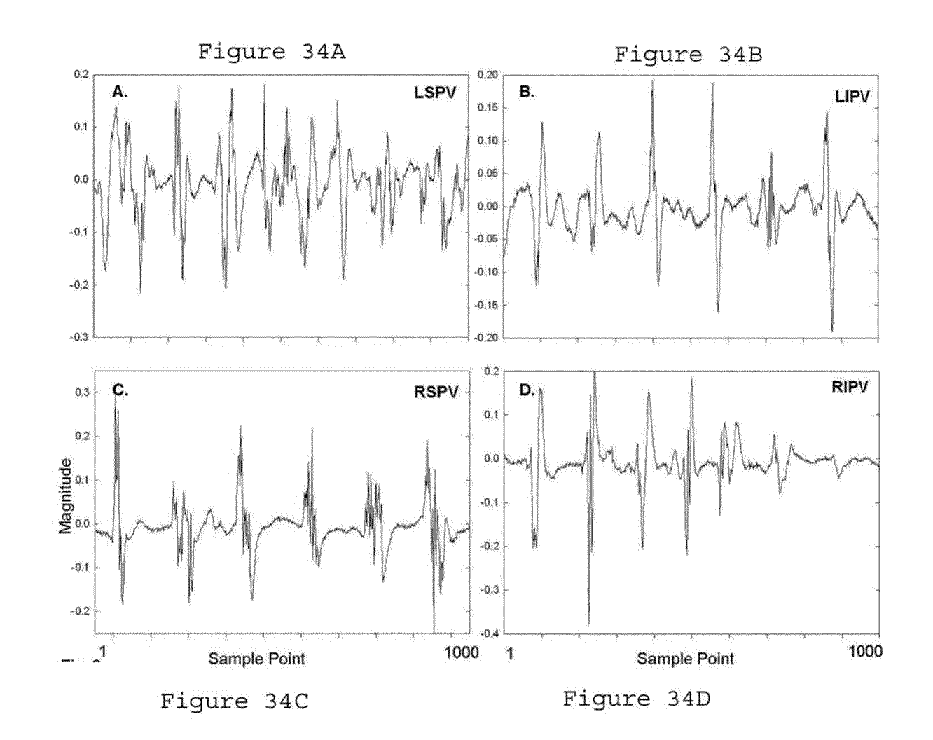

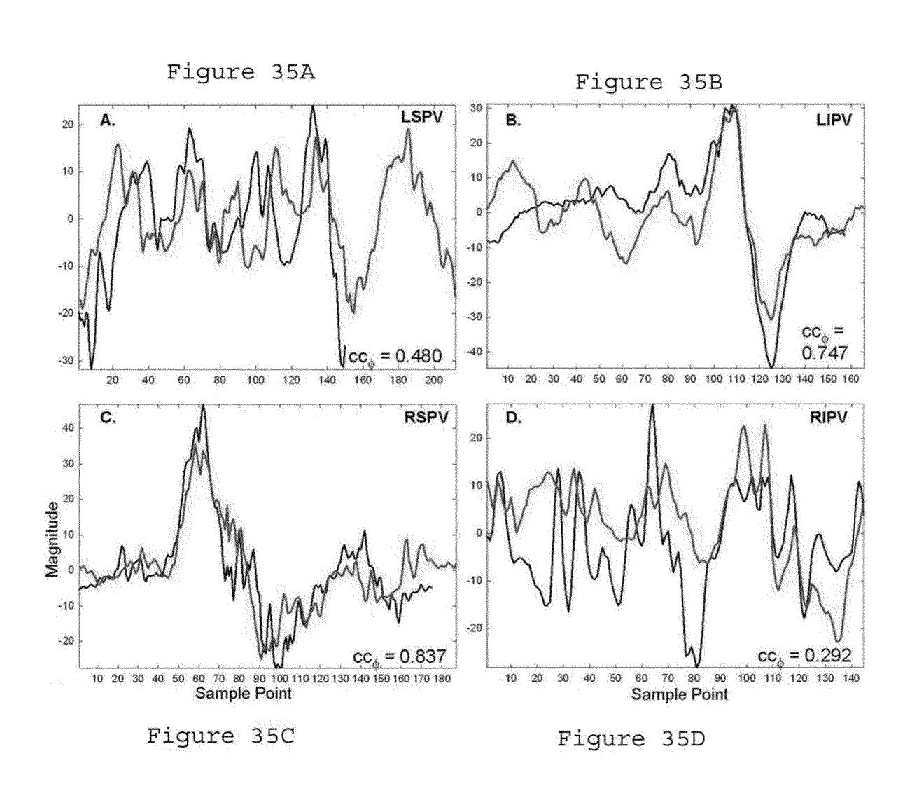

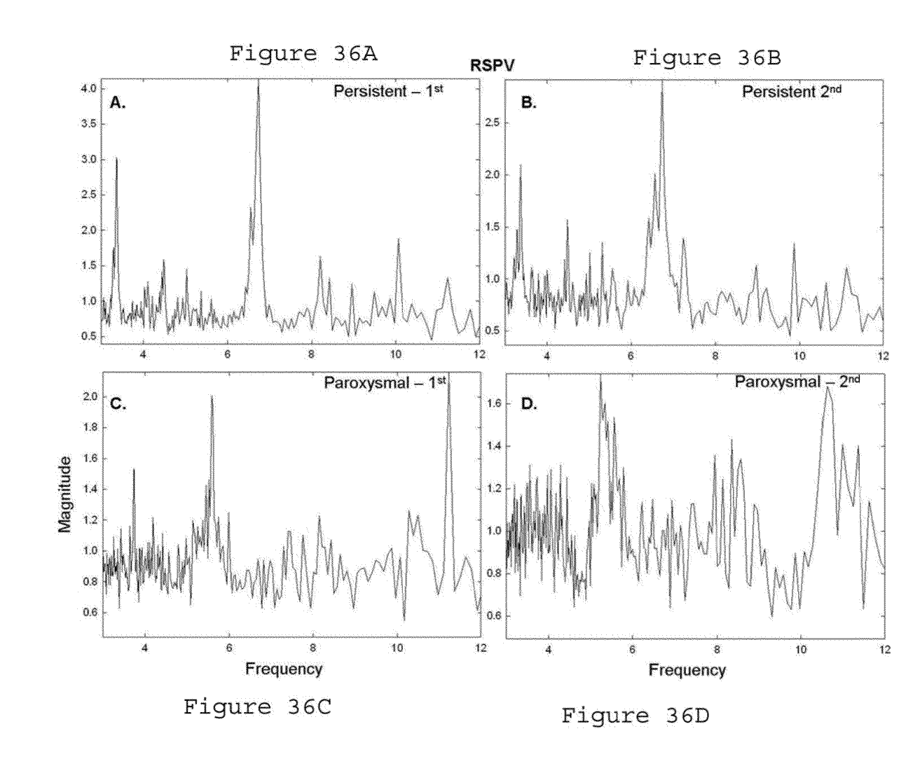

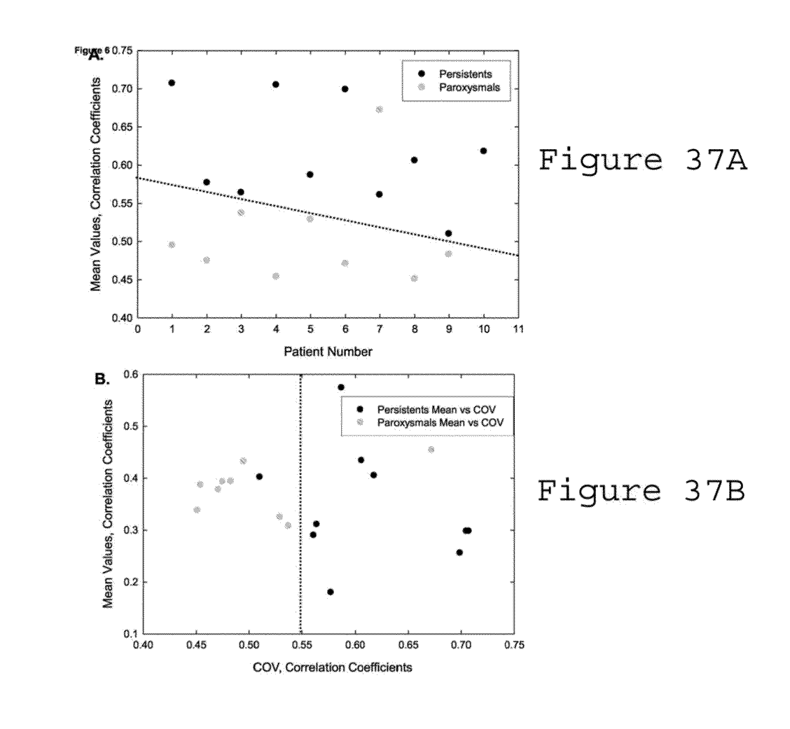

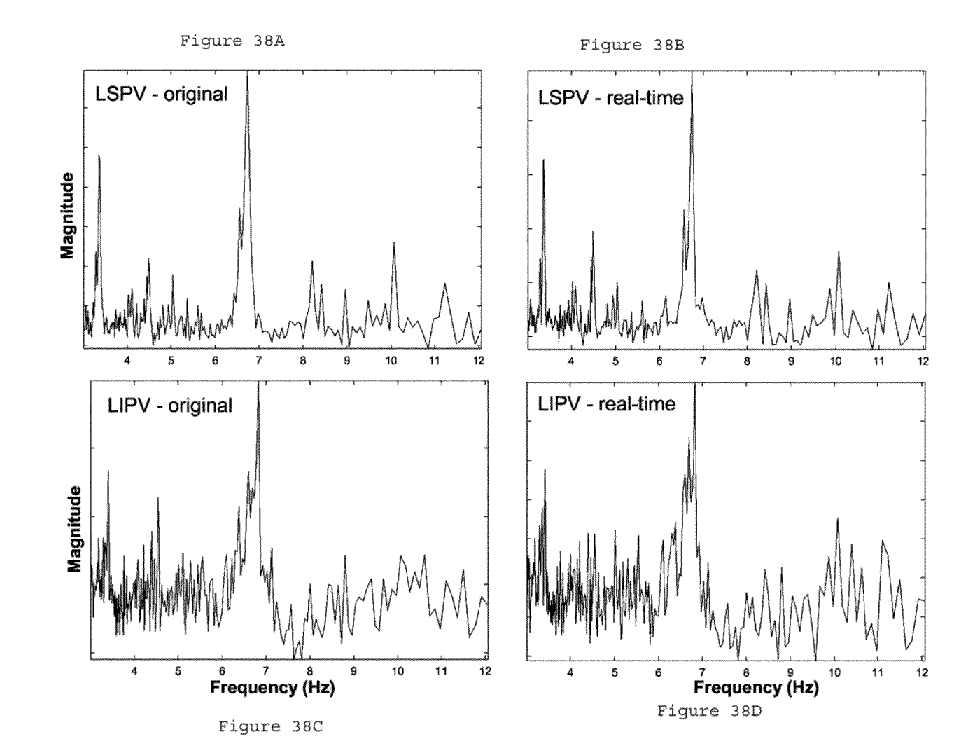

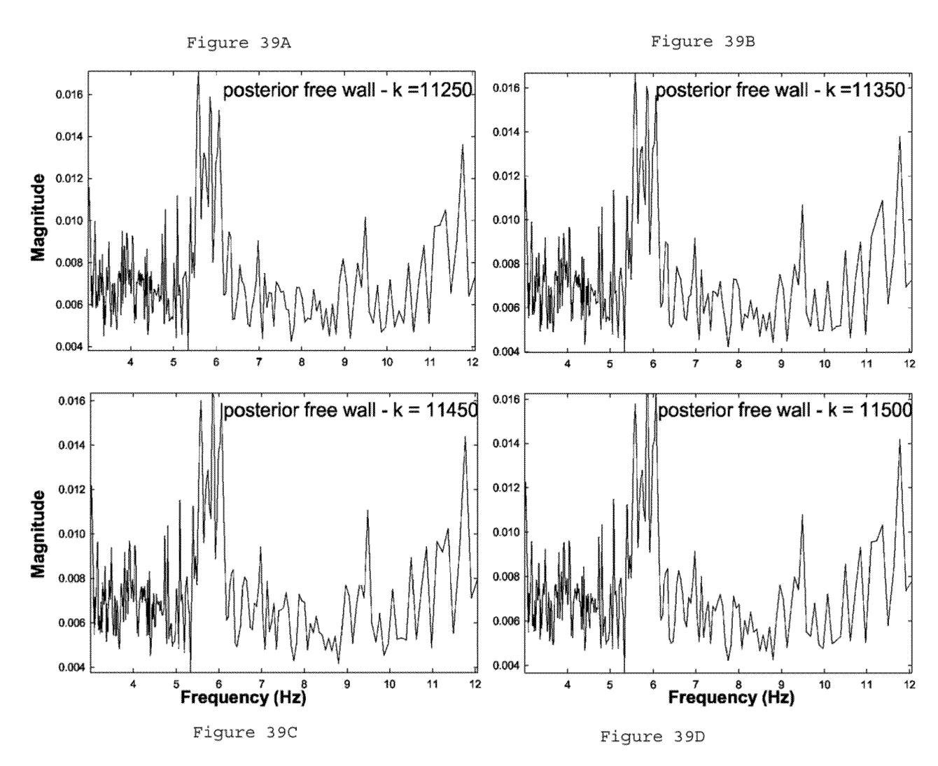

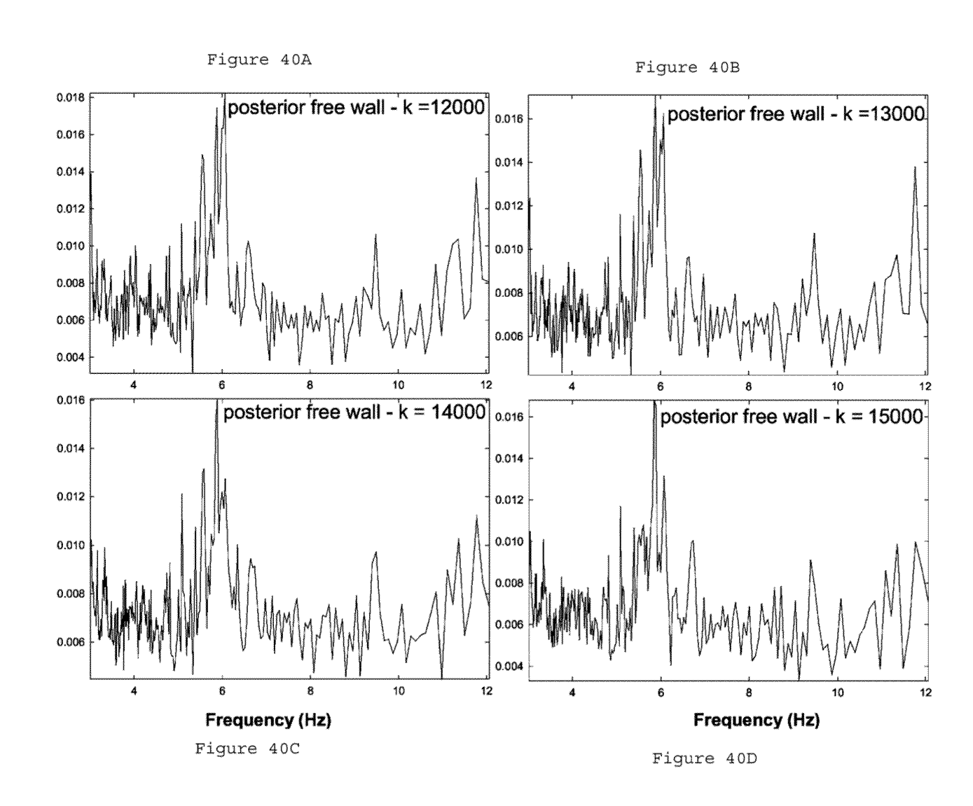

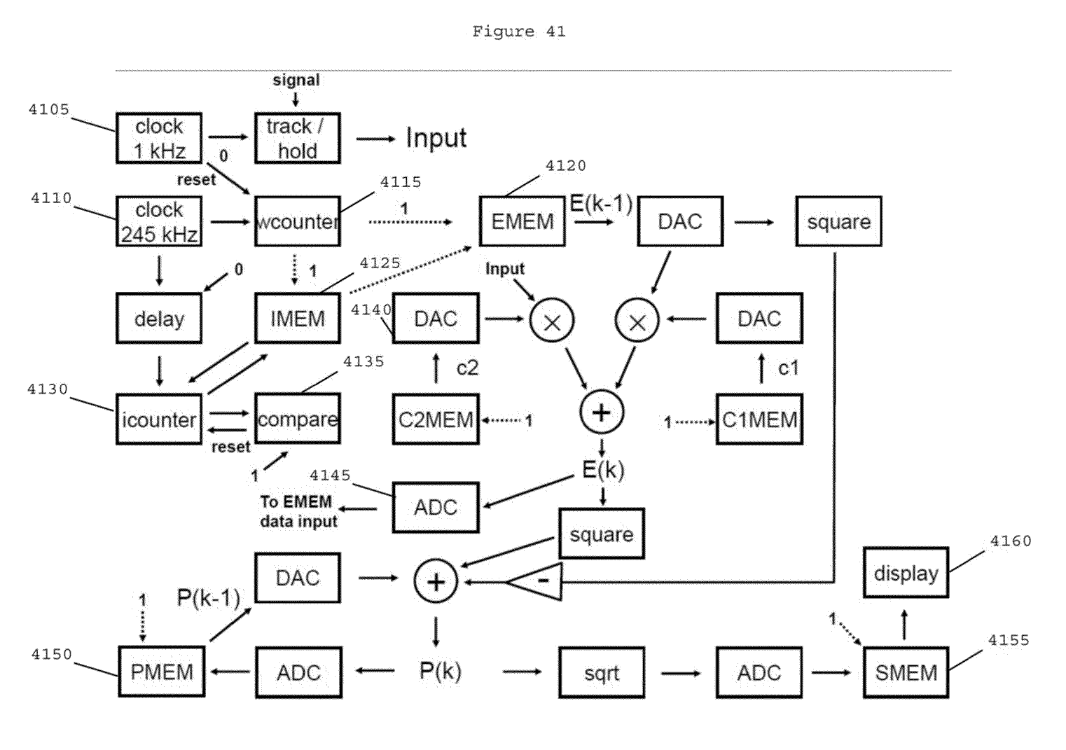

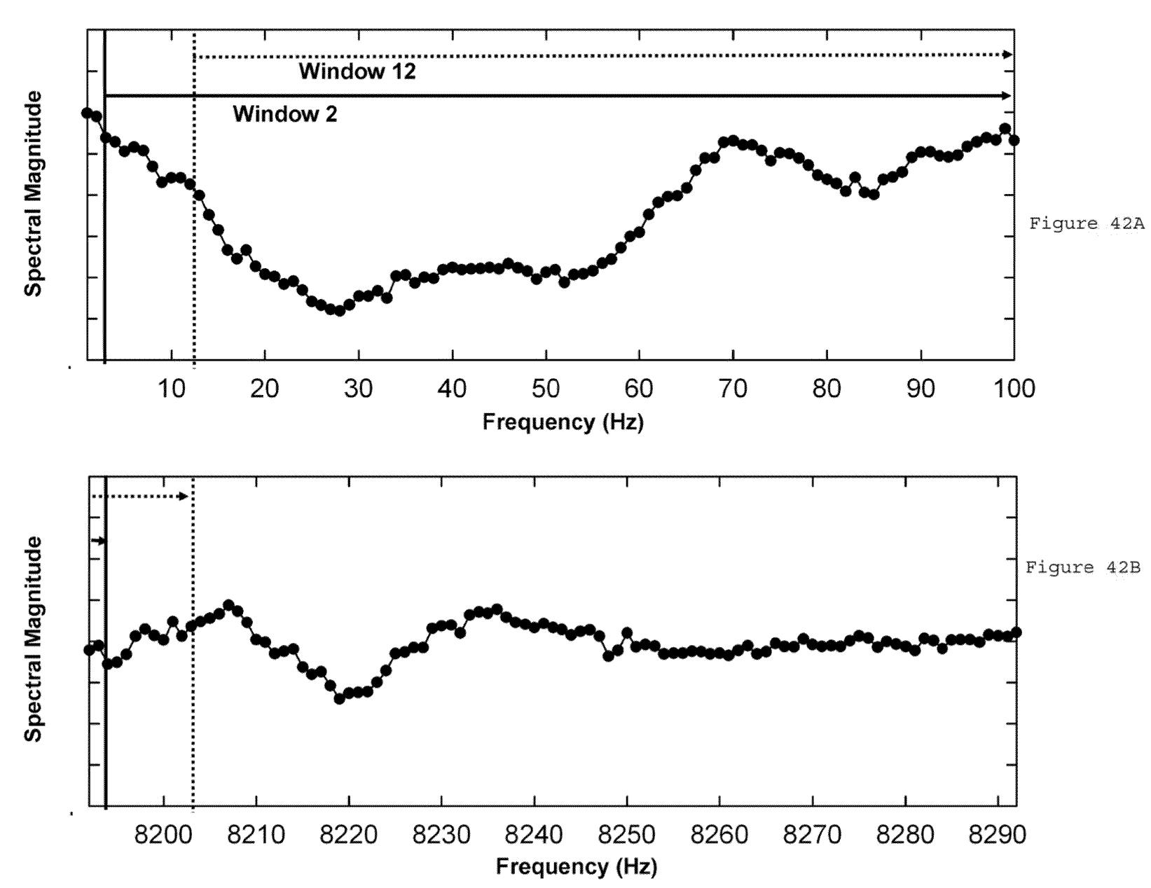

For random noise added with SD=±16 ms, the mean absolute difference in DF before versus after addition of a random noise vector can be, for example, about 0.35+0.02 Hz for Fourier spectral analysis versus about 0.09±0.05 Hz for ensemble spectral analysis (e.g., p<0.001). For random noise added with SD=±32 ms, the mean absolute difference in DF before versus after addition of a random noise vector can be, for example, about 0.68+0.10 Hz for Fourier spectral analysis versus about 0.53±0.13 Hz for ensemble spectral analysis (e.g., p=0.01). An example is shown in FIGS. 7A-7F for a CFAE signal from the anterior left atrial free wall of a paroxysmal AF patient. FIGS. 7A and 7B show, for example, the CFAE prior to and after addition of random noise with SD=±0.16 mV, while FIGS. 7C-7F show exemplary graphs of the corresponding Fourier and ensemble averaging spectra. In each spectrum, the DF is noted by an asterisk. After noise addition, the DF peak can be the third highest in the Fourier spectrum (e.g., FIG. 7D) but it can remain the highest peak in the ensemble averaging spectrum (e.g., FIG. 7F). Thus, as shown in FIGS. 7A-7F and Table 1, the DF peak in ensemble spectral analysis can be less affected by, and more robust to, random white additive noise might occur due to motion artifact, electrical component oscillation, and/or broken wire leads.

### Exemplary CFAE Reconstruction

An example of the Fourier basis vectors a w constructed from e_{w} with 1st and 10th highest power is shown, for example, in the graphs of FIGS. 8A and 8B from a paroxysmal CFAE signal acquired from the LIPV. The corresponding exemplary ensemble basis vectors for this same signal are shown, for example, in the exemplary graphs of FIGS. 8C and 8D. As the Fourier basis can typically be general and sinusoidal, the estimates can approximate the signal with relatively large error (e.g., FIGS. 8A and 8B). However the exemplary ensemble basis can be data-generated, and can be constructed from the first moment of the signal, so that it can be more estimative of the actual signal even when, for example, only the single most important basis vector can be used (see FIG. 5C). There can be substantial overlap with the actual CFAE trace when 10 basis vectors can be used for reconstruction (e.g., FIG. 8D). For the 12 reconstruction vectors, the root MSE averaged about 1.13±0.07 mV for Fourier versus about 0.98±0.10 mV for ensemble (e.g., p<0.001). The reconstruction error can be also lower for ensemble versus Fourier for each individual reconstruction using 1-12 bases (e.g., p<0.002).

The statistical relationships are illustrated, for example, in the exemplary graphs of FIGS. 9A-9C. The mean error in reconstruction for ensemble averaging can decrease more rapidly as compared with Fourier (e.g., FIG. 9A). The standard deviation in the reconstruction error for the CFAE is shown, for example, in FIG. 9B. The standard deviation can fall off rapidly for ensemble averaging and can increase rapidly for Fourier. At ≧3 basis vectors, the standard deviation in reconstruction error can be lowest for ensemble averaging. This means that the ability of ensemble averaging to consistently reconstruct CFAEs (e.g., FIG. 9B) with a similarly minimal level of error (e.g., FIG. 9A) can be mostly improved as compared with Fourier reconstruction. Similarly, the coefficient of variation, which can be the standard deviation divided by the mean (e.g., FIG. 9C), can fall off for ensemble average reconstruction but it actually increases for Fourier reconstruction.

### Exemplary Single Driver Test

The 5 Hz transient described above is shown, for example, in FIG. 10A and its addition to a CFAE is shown, for example, in FIG. 10B, trace 1022. For comparison, the original CFAE is shown as trace 1020 in the exemplary graph of FIG. 10B and it can be the same trace as in FIG. 1A. The Fourier and ensemble average power spectra are shown, for example, in FIGS. 10C and 10D, respectively. Although both spectra show a DF at about 7.5 Hz and a smaller peak at about 3.9 Hz (e.g., which can be generated by an independent driver), only the ensemble average power spectrum, for example, indicates presence of the artificial transient at 5 Hz (noted by *; with super- and sub-harmonics noted by **). For all CFAEs, the 5 Hz transient was identified, for example, in 216/216 ensemble averaging spectra (e.g., 100%) but was only present, for example, in 82/216 Fourier spectra (e.g., 38.0%). Additional examples are provided, for example, in the exemplary graphs of FIGS. 11A-11C. In each pair of Fourier and ensemble spectra, both have the same DF, for example, in the range 3-10 Hz. However, the 5 Hz transient can be evident in the ensemble averaging spectra (again noted by *; with super- and sub-harmonics noted by **). Thus, ensemble averaging but not Fourier spectral analysis can be sensitive to the presence of far-field and/or low-power drivers which affect CFAE over short intervals.

### Exemplary Improved Frequency Resolution for Characterization of CFAE

A graph of an exemplary power spectrum using the new spectral estimation technique is shown in the exemplary graph of FIG. 19A. Note that the highest frequency resolution occurs at lower frequencies due to the 1/w relationship of resolution to frequency for this method. By comparison, the Fourier power spectrum can be uniform in resolution across the range (e.g., FIG. 19C). FIGS. 19B and 19D show close-ups of the respective spectra in the range of the synthesized components. The actual synthesized components have frequencies of about 5.34 Hz (e.g., ω+γ=183 sample points at 977 Hz sampling rate) and about 5.43 Hz (e.g., ω=180 sample points), noted by vertical bars at the tops of FIGS. 19B and 19D. The two components can be correctly resolved by the new exemplary technique (e.g., FIG. 19C), that can be, w=ω and w+α+γ. However, Fourier analysis does not resolve at this component spacing (e.g., FIG. 19D). In FIGS. 20A-20D, using the same CFAE and with the high frequency remaining at about 5.43 Hz (e.g., ω=180 sample points), the exemplary graphical result is shown for γ=19 when the low frequency can be about 4.91 Hz (e.g., ω+γ=199 sample points). The spectrum and close-up using an exemplary embodiment of the method according to the present disclosure are shown in FIGS. 20A-B, and the frequency components are readily resolved as shown in an exemplary graph of FIGS. 19A-B. The Fourier spectrum is shown in FIGS. 20 C-D and now distinct peaks appear (see FIG. 20D), meeting the exemplary criteria set forth above. This was the exemplary minimum distance γ at which two corresponding Fourier spectral peaks met the criteria, and therefore the resolution for the Fourier spectrum. The measurements for Sw+α, Sw, Sminn and b are shown.

### Exemplary Identification of Recurring Patterns in Fractionated Atrial Electrograms

FIGS. 21A-21D illustrate exemplary graphs of signals and additive exemplary interferences. Identical scales can be used in all of FIGS. 21A-21D. For example, FIG. 21A illustrates a CFAE from the right superior pulmonary vein ostia in a paroxysmal AF patient. In FIG. 21B, an exemplary CFAE graph is illustrated from the anterior left atrial free wall in another paroxysmal AF patient. Both signals have mostly continuous activation, and the large deflections have different shape and timing at each occurrence. Only about 1000 of about 8192 sample points are shown for clarity (e.g., approximately 1 second), although about 8192 points were used for the calculations described in the Methods. The signals illustrated in the exemplary graphs of FIGS. 21A and 21B can be used as patterns, which can be made to occur, for example, five and four times, respectively, in the final data set of, for example, about 214 signals used for analysis. Examples of additive interference are shown in the exemplary graphs shown in FIGS. 21C and 21D. The interferences are each a combination of two AF signals unrelated to signals shown in FIGS. 21A and 21B. The same or similar exemplary patterns after addition of the interferences are shown in the corresponding exemplary graphs shown in FIGS. 21E and 21F. With the additive interferences, the original signals can be almost completely unrecognizable visually. Most of the original signal deflections can be masked by interference.

The exemplary spectrum of the combined exemplary patterns illustrated in FIGS. 21A and 21B is shown in FIG. 22A in the range of 1-12 Hz, where pattern A (signal x)+pattern B (signal y) form the combined signal z. For example, several prominent peaks are shown in the exemplary spectrum of z, likely related to individual components of the two signals. The transform coefficients of x and y with respect to the basis vectors of z were separately calculated and then added together and plotted as a trace 2202 in FIG. 22B, shown with exemplary overlapping z spectrum 2204 shown in FIG. 22A. There can be perfect overlap. In contrast, when the spectral signatures of two other signals not related to x or y can be obtained with respect to z, their magnitude throughout the frequency range can be relatively small and the transform coefficients can be both positive and negative (see exemplary FIGS. 22C and 22D; same or similar 5-unit range in ordinate scale as shown in FIGS. 22A and 22B).

To further elucidate the exemplary process, when the spectral signatures of x and y with respect to z are separately plotted (as shown in FIGS. 23A and 23B, respectively), there can be similarities to the z spectrum shown in FIG. 22A. Therefore, exemplary elements of the z spectrum (e.g., FIG. 22A) are maintained in the spectral signatures of x and y (see exemplary graphs of FIGS. 23A and 23B, respectively), which can suggest that the Euclidean distances between them will be relatively small. In contrast, the elements of the z spectrum are not maintained in the spectral signatures of random interferences, such as those shown in exemplary graphs of FIGS. 22C and 22D, which can suggest that the Euclidean distances between them can be relatively large. Finally, the spectral signatures of x and of y with respect to z, shown again as traces 2210 in exemplary graphs of FIGS. 23C and 23D, can be similar, but not the same, as the spectra of x and y, which are denoted as traces 2212 shown in FIGS. 23C and 23D. Based on the exemplary graphs shown in exemplary graphs of FIGS. 22A-22D and 23A-23D, the spectral signatures of x and y with respect to z can be related to the actual frequency content in signals x and y. However, the x and y spectra do not resemble each other since they can be uncorrelated.

The Euclidean distance between the spectral signatures of each of 214 signals with differing additive interference, versus the spectrum of the mean signal containing two patterns A and B, is shown in an exemplary graph of FIG. 24A. A number of downward projections are illustrated in FIG. 24A, which can indicate increased correlation and possible instances of pattern recurrence. If the lower threshold can be used, nine possible instances of repetitive patterns can be selected (e.g., shown in binary form in an exemplary graph of FIG. 24B). When the upper threshold can be used, eleven possible instances of repetitive patterns can be selected (e.g., shown in binary form in an exemplary graph of FIG. 24C). The detected pattern type (e.g., A or B) or non-pattern (n) is shown at the bottom of FIGS. 24B and 24C. The selection of a threshold higher along the ordinate axis in the Euclidean distance graph shown in FIG. 24A can facilitate the detection of more candidate patterns. However, whatever threshold can be used, to determine and identify the presence of actual recurring patterns necessitates can need the last step at the lower right in the pattern recognition flow diagram of FIG. 1, (e.g., the exemplary spectral signatures of the signals selected by threshold shown in FIGS. 24A-24C can be compared). Due to the exemplary constructing of the signals with the exemplary interference, as described herein, each downward projection shown in FIGS. 24B and 24C can represent a set of three exemplary successive signals with pattern, of which the middle was used for an exemplary statistical calculation.

The Euclidean distances for the exemplary pairings of spectral signatures using the upper exemplary threshold shown in FIG. 24A (e.g., shown in binary form in FIG. 24C) are provided in Table 2. The first column and first row in Table 2 indicate the actual pattern that was selected by the upper threshold in FIGS. 24A-24C, and correspond to the sequence shown in FIG. 24C. Since the two patterns A and B occurred only nine times in the sequence, two of the selections shown in the exemplary graphs of FIGS. 24A-24C, top threshold, were of non-patterns (n). In the case of the pairing of a spectral signature from a particular signal with itself, the Euclidean distance can be zero (e.g., main diagonal in Table 2). There can be an exemplary symmetry above and below the main diagonal (e.g., half the table can be redundant). Smaller values in Table 2 can indicate shorter Euclidean distances, for example, spectral signatures that can be more similar. The Euclidean distances can be small for spectral signatures of pattern A embedded in one interference versus pattern A embedded in another interference, and similarly for pattern B embedded in one interference versus pattern B embedded in another interference. The Euclidean distances can be large for spectral signatures of pattern A versus pattern B embedded in interference, for spectral signatures of patterns A and/or B embedded in interference versus non-patterns (e.g., interference only), and for spectral signatures of non-pattern versus non-pattern. Thus, the exemplary patterns and non-patterns with interference can be distinguished based on a threshold level Euclidean distance.

Based on the information provided in Table 2, an exemplary threshold level of about 0.105 normalized units can be estimative to distinguish patterns and non-patterns with 100% sensitivity and specificity. Such exemplary pairings above about 0.105 can indicate that the same pattern may not be present on both signals, while pairings less than or equal to about 0.105 can indicate the same pattern being present on both signals. Using the exemplary threshold of about 0.105 for clustering and classification in, for example, all 10 trials, the exemplary results are shown in Table 3, left-hand columns. For 10 trials, the sensitivity to correctly detect and distinguish patterns was about 96.2%. The specificity to exclude non-patterns was about 98.0%. For the test of interference+noise, a threshold value for TH2 of about 0.132 was found to be efficacious in a test trial, and was then used in all trials. The exemplary results are shown in Table 3, right-hand columns, with mean values of about 89.1% for sensitivity and about 97.0% for specificity. Thus, the exemplary embodiment of the technique, method and system, according to the present disclosure, can be nearly as efficacious for classification when random noise as well as interference is added to CFAE.

TABLE 2

| Pattern | A | n | A | B | A | A | A | B | B | n | B |
|---|---|---|---|---|---|---|---|---|---|---|---|
| A | 0.000 | 0.142 | 0.056 | 0.133 | 0.092 | 0.074 | 0.065 | 0.128 | 0.135 | 0.221 | 0.135 |
| n | 0.142 | 0.000 | 0.151 | 0.149 | 0.166 | 0.123 | 0.131 | 0.188 | 0.156 | 0.143 | 0.122 |
| A | 0.056 | 0.151 | 0.000 | 0.136 | 0.097 | 0.068 | 0.068 | 0.143 | 0.145 | 0.214 | 0.138 |
| B | 0.133 | 0.149 | 0.136 | 0.000 | 0.167 | 0.127 | 0.144 | 0.101 | 0.096 | 0.185 | 0.063 |
| A | 0.092 | 0.166 | 0.097 | 0.167 | 0.000 | 0.095 | 0.101 | 0.196 | 0.206 | 0.271 | 0.151 |
| A | 0.074 | 0.123 | 0.068 | 0.127 | 0.095 | 0.000 | 0.082 | 0.156 | 0.124 | 0.191 | 0.116 |
| A | 0.065 | 0.131 | 0.068 | 0.144 | 0.101 | 0.082 | 0.000 | 0.156 | 0.151 | 0.212 | 0.152 |
| B | 0.128 | 0.188 | 0.143 | 0.101 | 0.196 | 0.156 | 0.156 | 0.000 | 0.105 | 0.241 | 0.102 |
| B | 0.135 | 0.156 | 0.145 | 0.096 | 0.206 | 0.124 | 0.151 | 0.105 | 0.000 | 0.161 | 0.104 |
| n | 0.221 | 0.143 | 0.214 | 0.185 | 0.271 | 0.191 | 0.212 | 0.241 | 0.161 | 0.000 | 0.168 |
| B | 0.135 | 0.122 | 0.138 | 0.063 | 0.151 | 0.116 | 0.152 | 0.102 | 0.104 | 0.168 | 0.000 |

A—pattern A.
B—pattern B.
n—nonpattern.
There is symmetry about the main diagonal.

TABLE 3

| trial # | sen: int | spe: int | sen: int + n | spe: int + n |
|---|---|---|---|---|
| 1 | 100.0 | 100.0 | 91.1 | 100.0 |
| 2 | 97.8 | 100.0 | 97.8 | 95.0 |
| 3 | 93.3 | 100.0 | 82.2 | 100.0 |
| 4 | 95.6 | 100.0 | 88.9 | 100.0 |
| 5 | 93.3 | 100.0 | 88.9 | 100.0 |
| 6 | 91.1 | 80.0 | 88.9 | 75.0 |
| 7 | 95.6 | 100.0 | 88.9 | 100.0 |
| 8 | 97.8 | 100.0 | 91.1 | 100.0 |
| 9 | 97.8 | 100.0 | 84.4 | 100.0 |
| 10 | 100.0 | 100.0 | 88.9 | 100.0 |
| mean | 96.2 ± 3.0 | 98.0 ± 6.3 | 89.1 ± 4.1 | 97.0 ± 7.9 |

sen—sensitivity,
spe—specificity,
int—interference,
n—noise

### Exemplary Spectral Profiles of Complex Fractionated Atrial Electrograms

For example, no significant changes occurred in any parameter from the first to second recording sequence. For both exemplary sequences, MPS and SPS were significantly greater, and DF and ADF were significantly less, in paroxysmals versus persistents. The MPS and ADF measurements from ensemble spectra produced the most significant differences in paroxysmals versus persistents (e.g., P<0.0001). DF differences were less significant, which can be attributed to the relatively high variability of DF in paroxysmals. The MPS was correlated to the duration of uninterrupted persistent AF prior to electrophysiologic study (e.g., P=0.01), and to left atrial volume for all AF (e.g., P<0.05)

### Exemplary Discussion

According to certain exemplary embodiments of the present disclosure, for example, a data-driven transform can be provided for application to CFAE signals. The basis can be constructed, for example, from the ensemble averages of signal segments and can be found to be orthogonal except for small integer-multiple relationships. The power in each ensemble average can be equivalent to the projection of the signal onto the corresponding basis (e.g., exemplary Eq. (15)). The relationship of the ensemble spectrum to the autocorrelation spectrum and to the Fourier power spectrum can be shown. While the autocorrelation spectrum can be based on correlation at a single lag w, the ensemble and Fourier power spectra can be based on correlation at multiple lags w, 2w, . . . , nw. During construction of the ensemble spectrum, the autocorrelation function at lags can be averaged, as compared to the Fourier power spectrum which can typically be a sinusoidal curve fitting of the autocorrelation function. Several tests can be used to compare the efficacy of the Fourier transform, versus transformation using ensemble averaging, for representation of CFAE signal components.

At several levels of additive noise and interference, the highest peaks in the ensemble spectrum can corresponded to the frequencies of three synthetic drivers with higher accuracy as compared to Fourier spectral analysis (e.g., p<0.001). Similarly, when random noise corrupted actual CFAE signals, the ensemble spectrum can be more accurate than Fourier in representation of the DF (e.g., p<0.01). The ensemble basis can be found to be useful for representation of the signal morphology of the three independent synthetic drivers. When only interference was added, the top three ranked basis vectors in order of greatest power can correspond to the independent driver morphology. When phase noise (e.g., jitter) was added, the top three ranked basis vectors can correspond to driver morphology, but with some smoothing. When a single low-power, short duration component was added as would simulate a distant driver, it can be evident as a distinct peak in all 216/216 ensemble averaging spectra but in only about 82/216 Fourier spectra. Further, when both Fourier and ensemble were used for reconstruction of actual CFAE signals, the ordered ensemble basis from 1-12 vectors can be more accurate as compared with Fourier for representation (e.g., p<0.001). Thus, it can be found that the exemplary transform can be more efficacious for representation of independent generator frequencies and CFAE morphologies as compared to the Fourier transform.

### Exemplary Computational and Mathematical Considerations

Although ensemble analysis can be robust to noise and jitter, to further reduce their effect on signal analysis, the inner product between the spectrum and a model can be used for gradual, adaptive update (see, e.g., Reference 25) or alternatively, finite differences can be used for adaptation (see, e.g., Reference 26). When computing and/or determining the DF of atrial fibrillation signals, variation by as much as about 2.5 Hz can occur over a time interval of a few seconds; hence tracking with time-frequency methods can be required for accurate analysis (see, e.g., References 27 and 28). Since ensemble averaging can be a form of autocorrelation, a minimum sequence length of two cycles of the periodic signal can be needed for construction of the frequency spectrum (e.g., which can result in a very course estimation). To include low frequency activity to a lower limit of about 2 Hz, as can be done in accordance with certain exemplary embodiments of the present disclosure, a window of at least about 1000 ms (e.g., about 1 s) can preferably be used. Any such measurement can be updated by shifting the analysis window, for example, by about 100-150 ms steps, to describe the time-frequency evolution of the signal (see, e.g., Reference 29).

To reduce error when any such short sequences can be utilized for analysis, a model-based approach for update of the spectral profile can be implemented (see, e.g., Reference 30). In an exemplary study, the DF computed by Fourier analysis was compared with the mean, median and mode activation rate, as obtained by electrogram marking, to determine efficacy (see, e.g., Reference 10). However, as stated in that study, DF does not typically specifically reflect activation rate, and therefore can typically be only an approximate measure, with a level of uncertainty. For this reason, adding artificial drivers at specific frequencies, as well as to analyzing the degradation of actual DFs in CFAE when random noise can be introduced, can act as tests to compare the Fourier versus ensemble methods. In each of the exemplary tests of DF measurement, the highest peak in the spectral range can be selected as the DF. The more accurate selection of DF in presence of noise and interference by ensemble analysis can in part be due to increased spectral power in the fundamental frequency relative to sub- and super-harmonics as compared with Fourier (see, e.g., Reference [18]).

Knowledge of the mechanisms for onset and maintenance of atrial fibrillation can be scant or limited due to the difficulty in quantitative assessment of the CFAE signal with an exemplary Fourier method, which can distort the signal during preprocessing and can suffer from phase noise degradation of the estimate (see, e.g., Reference 31). By devising a data-driven frequency transform, independent drivers can be successfully extracted and characterized by both frequency and morphologic measurements. The transform can be further developed for clinical use by activation mapping of the substrate during AF in patients, identifying independent sources in the maps (e.g., focal or reentrant), and determining the correspondence of these to the most important ensemble basis vectors and frequency components. It can be believed that ablation lesions at these sources can best prevent reinduction of AF (see, e.g., Reference 5 and 6). Simulations have suggested that sinusoidal electric fields can be important for excitation of cardiac tissue (see, e.g., Reference 32). If such sinusoidal generators exist in nature, they can be efficiently represented by the Fourier transform, which can typically be based upon sinusoidal components, but also by the ensemble basis from which any such components can be readily reconstructed. Certain exemplary embodiments of the present disclosure can include retrospective analysis of nonsynchronous CFAE when comparing the ensemble averaging method with the Fourier transform.

Other clinical research can project the AF signals onto ensemble space using exemplary Eq. (15) for solution of two- and multiple-class problems. A plot of x^{T}a_{w} versus w can be a rendition of the ensemble power spectrum. One way to express differences between CFAE can be based on the difference in Euclidean distance in n ensemble space, where n can be given in exemplary Eq. (5), and can equal the number of points in the power spectrum. This can be computed as the square root of the sum of squares difference in corresponding points between power spectra. Suppose for example that many CFAE recordings can be obtained simultaneously from the left atrium. The ensemble spectrum of each can be compared with its nearest neighbors, with the difference in spectra for all neighbors averaged. If this can be done for the CFAE, areas with small spectral difference can suggest presence of a driver, and/or other homogeneous regions, where spectral characteristics can be similar, while areas with large spectral difference can suggest the presence of substrate heterogeneity and/or boundary areas where multiple drivers compete. Another exemplary method of classification can be to sum all CFAE in a neighborhood region, compute the ensemble basis, project each CFAE onto the global basis, and cluster and classify according to the position of each point in n ensemble space. Elsewhere, ensemble spectra have been used to analyze the DF of ventricular tachyarrhythmias (see, e.g., Reference 29) and to assess videocapsule endoscopy images for estimation of small bowel motility (see, e.g., Reference 33), as described in further detail below. Thus, this exemplary new transform can have wider application for clinical data analysis.

### Exemplary Clinical Procedure and Data Acquisition—Videocapsule Endoscopy

Exemplary clinical data was collected implementing/utilizing certain exemplary embodiments of the present disclosure. Patients were evaluated, for example, at Columbia University Medical Center, New York. Retrospective videocapsule endoscopy data was, for example, obtained from ten celiac patients on a regular diet or within a few weeks of starting a gluten-free diet. In these patients, the diagnostic biopsy taken while on a regular diet, showed Marsh grade II-IIIC lesions. Informed consent was obtained prior to videocapsule endoscopy. Indications for this procedure included, for example, suspected celiac disease or Crohn's disease, iron deficient anemia, obscure bleeding, and chronic diarrhea. Patients had serology and biopsy-proven celiac disease. These patients were being subsequently evaluated by videocapsule endoscopy because they were considered to have complicated disease such as abdominal pain unexplained by previous evaluation. Exclusion criteria included, for example, patients under 18 years of age, those with a history of or suspected small bowel obstruction, dysphagia, presence of pacemaker or other electromedical implants, previous gastric or bowel surgery, serum IgA deficiency, pregnancy, and chronic NSAID use or occasional NSAIDs use during the previous month. Preferably, complete videocapsule endoscopy studies, reaching the colon, were used for analysis. The retrospective analysis of videocapsule endoscopy data was approved by the Internal Review Board at Columbia University Medical Center.

The PillCamSB2 videocapsule (e.g., Given Imaging, Yoqneam, Israel) was utilized to obtain the small bowel images in the study groups. The system typically includes a recorder unit, battery pack, antenna lead set, recorder unit harness, battery charger, recorder unit cradle and real-time viewer with cable. The capsule can acquire two digital frames per second and can be a single-use pill-size device (see, e.g., Reference [15A]). For each patient undergoing the procedure, abdominal leads were placed, for example, in the upper, mid, and lower abdomen, and a belt that contained the data recorder and a battery pack was affixed around the waist. The subjects swallowed the videocapsule, for example, with radio transmitter in the early morning with approximately 200 cc of water, after a 12 hour fast without bowel preparation. Subjects were allowed to drink water, for example, 2 hours after ingesting the capsule, and to eat a light meal after 4 hours. The recorder received radioed images that were transmitted, for example, by the videocapsule as it passed through the gastrointestinal tract. The capsule reached the caecum in the participants from which retrospective data was used in this study. The belt data recorder was then removed, and the data was downloaded, for example, to a dedicated computer workstation. Videos were reviewed and interpreted, for example, by an experienced gastroenterologist using the HIPAA-compliant PC-based workstation equipped with Given Imaging analysis software that was also used to export videos for further analysis. For example, video clips of 200 frames each acquired from the small intestine for each patient by the patients' physicians were analyzed retrospectively.

The retrospectively obtained patient video clips were then transferred to a dedicated PC-type computer for quantitative analysis. From each RGB color video clip, grayscale images (e.g., 256 brightness levels, 0=black, 255=white) with an image resolution of 576×576 pixels, were extracted, for example, using Matlab Ver. 7.7, 2008 (e.g., Mathworks, Natick Mass.). One sequence of 10-20 frames was extracted from each video clip, for example, in which air bubbles and opaque extraluminal fluids were absent. Each sequence of N frames from 10-20 was repeated to form a series 200 frames long, typical for video clip quantitative analyses. Thus the total number of repeating sequences of length N in the synthesized 200 frame series was 200/N. Additionally, a single frame from one celiac patient in which air bubbles was the dominant feature in the image, selected at random, was extracted for use as an extraneous image frame.

### Exemplary Improved Frequency Resolution for Characterization of CFAE

According to one exemplary embodiment of the present disclosure, a comparison was made between the ability to resolve two closely-spaced frequency components in the physiologic range of interest using Fourier power spectral analysis, versus a new exemplary technique that utilizes signal averaging. The exemplary synthesized closely spaced frequency components and two exemplary additive interferences were selected at random from a set of, for example, about 216 CFAE. The values for digital sampling rate (e.g., about 977 Hz) and sequence length (e.g., N=8192, at about an 8.4 s sequences) can be typical of those used for frequency analysis of CFAE obtained during clinical EP study. Tests were made in the range of about 3-10 Hz, the electrophysiologic range for evaluation of atrial electrical activity. From 105 tests, the mean resolving power of Fourier versus the new technique (e.g., about 0.29 Hz versus about 0.16 Hz; p<0.001), were higher than the theoretical values but in accord with the presence of large interferences that could act to mask the frequency components. In 13/105 trials, interference masked frequency components in the Fourier power spectrum. By comparison, this occurred in only for example, 4/105 trials using the new exemplary technique. The error in estimating the synthesized components was about ±0.023 Hz using Fourier versus about ±0.009 Hz using the exemplary technique, system and method according to an exemplary embodiment of the present disclosure (e.g., p<0.001).

The use of the exemplary embodiment of the exemplary technique, system and method, according to the present disclosure, compared to Fourier, produced an improved frequency resolution and improved compression with less loss of resolution. For a given time data, the exemplary embodiment of the technique, system and method, according to the present disclosure, can provide, for example, double the frequency resolution, as compared to that that uses the Fourier transform. A decrease in the time so as to produce the same or similar frequency resolution using the exemplary technique, system and method, according to the present disclosure, versus those using the Fourier transform provides a significant advantage in reducing and/or preventing errors that can occur over time. Exemplary procedures that use the exemplary technique, system and method, according to the present disclosure, can be performed in less time, for example, decreasing the fluoroscopy radiation received by a patient.

### Exemplary Identification of Recurring Patterns in Fractionated Atrial Electrograms

According to certain exemplary embodiment of the present disclosure, it can be possible utilize an exemplary transform to characterize recurring patterns in CFAE. First, for example, ensemble averages can be computed from signal segments of length w, repeated for all w in the frequency range of interest. From each ensemble average, an exemplary orthogonal basis vector can be constructed by repeating the ensemble average of length w for the entire signal length N. The inner product between basis vector and original signal can produce a transform coefficient, which can be the signal power at that frequency. The exemplary power spectrum can be a plot of the entire series of transform coefficients versus frequency. Exemplary transform coefficients resulting from the inner product of one signal with the basis vectors of another signal can take on negative as well as positive values, and can have an average level near zero if the signals can be uncorrelated. The correlation coefficients formed from correlated signal x with the basis vectors of z can be similar to the spectrum of x and can be termed the spectral signature. Transform coefficients can be used to detect two recurring patterns in a sequence of CFAE, embedded in interference and random noise, and to distinguish them from each other and from non-patterns. For example, no manual intervention was used except to set initial threshold levels of Euclidean distance for identification of correlated content, for example, for pattern extraction, and to distinguish the extracted patterns.

The spectral signature can be a graph of the correlated content between two signals in frequency space, which can be exploited for pattern recognition. If a series of signals can be averaged, and the basis vectors of the mean can be used to obtain the spectral signature of each individual signal, then there can be a correlation between the spectrum of the mean, and the spectral signature of the individual signal, when the individual signal contains a synchronous pattern that recurs within the series. By measuring the Euclidean distance between all individual signals having spectral signatures similar to the power spectrum of the mean signal, patterns contained in the sequence can be identified, distinguished from one another, and distinguished from non-patterns when the non-patterns can be mostly uncorrelated with respect to the mean signal. Thus, the exemplary technique, system and method, according to the present disclosure, can be used to automatically identify and distinguish repetitive patterns present in a series of signals, once threshold levels for the Euclidean distance estimate to detect candidate patterns, and to discern patterns, can be established. The determination of the exemplary patterns, if multiple patterns can be present, the patterns can also be discerned using a single threshold level, since the Euclidean distance will be short only with respect to members of the same class. Successful source pattern recognition can be useful in catheter ablation as an identification of a source pattern can provide for an area for best ablation.

### Exemplary Frequency Resolution for Characterization of CFAE

According to an exemplary embodiment of the present disclosure, by normalizing the CFAE spectra, it can be possible to compare the CFAE frequency patterns observed in longstanding, persistent AF to those present in acutely induced AF in patients whose arrhythmia can be clinically paroxysmal and whose baseline rhythm was sinus. Exemplary results can indicate that the CFAE recordings during acute onset AF in patients with paroxysmal AF had significantly larger mean and standard deviation in the normalized power spectra, suggesting, but not proving, the presence of more randomly varying activation sources in general. By comparison, CFAE spectra from longstanding AF patients had lower mean value and standard deviation of spectral peaks, as would be expected if the peaks were generated by more stable and stationary sources present in the atrial substrate. The exemplary results also indicate that the CFAE recordings during acute onset AF in patients with paroxysmal AF had significantly lower amplitude and frequency of the dominant peak. This can indicate a greater complexity in the power spectral profile of paroxysmal patients, which can likely be due to the presence of more peaks that can be greatly varying in height, with no single predominant tall peak in the spectrum.

### Exemplary Image Corruption

Each of the exemplary image series was corrupted, for example, by the following exemplary methods (e.g., one or multiple used at the same time):
1. Temporal Phase Noise: the 200 frame series was altered, for example, by removing 1-5 frames from the beginning or end of one of the repeating sequences comprising the series, and appending it to another of the sequences. This was done, for example, 2-3 times at random for each 200 frame series.
2. Spatial Phase Noise: each image in the 200 frame series was altered, for example, using a maximum row-by-row pixel rotation of m=1-20 pixels. The degree of pixel rotation was the same for each row in a particular image, but was varied randomly from one image to the next from 0 to m.
3. Addition of Random Noise: a series of X image frames were removed, for example, from the end of the 200 frame series and replaced with X white noise frames, where the number of frames removed was varied from X=0 to 180.
4. Addition of Bubble Image: 5-10 images were randomly removed, for example, from the 200 frame series and replaced with an image composed primarily of bubbles that did not belong in the series. The image used was the same for each of the ten 200 frame series that were analyzed.

The DP was calculated for each 200 frame series without any corruption and with imposition of one or more of the methods listed above (e.g., total of 20 trials for each series).

### Exemplary Spectral Analysis

Both the Fourier and the exemplary ensemble spectral analysis methods can be used for DP calculation. For analysis, the series of 200 grayscale brightness values at each pixel location can be treated, for example, as a signal. Each of these 576×576=331776 signals can be, for example, first set to mean zero. Then, the power spectrum (e.g., Fourier or the ensemble method) can be computed for each, and the average of all 331776 individual power spectra can be, for example, considered to be the videocapsule frequency spectrum. The tallest peak in the power spectrum can be taken as the DF, which can be related to the DP, based on a frame rate of 2 per second, as, for example:

DP=2./DF (22)

where DF can have units of Hz and DP can have units of seconds. All computation were done, for example, using a Lenovo x60 laptop computer, Windows XP Pro (e.g., Service Pack 3) operating system and Intel T2400 processor running at 1.83 GHz with 3 GB of RAM memory. Prior to Fourier spectral calculation, the exemplary 200 point data array can be smoothed using a Hann window of the form, for example:

a[k+1]=0.5*[1−cos(2πk/(n−1)],k=0,1, . . . n−1 (23)

where a[k] can be, for example, the weights by which the 200 point array can be multiplied. The windowed data can be then padded with about 56 zeros to form an array 28=256 points. Since the sample rate was about 2 frames/second, for example:

$$\begin{matrix}resolution = sample rate \text{/} signal length N \\ = ( 2 frames \text{/} s ) \text{/} 256 frames\end{matrix} \qquad ( 24 )$$

which can be, for example, 0.0078 Hz. The FFT was computed using the Intel Visual FORTRAN Compiler 9.0 Build Environment for 32-bit applications (e.g., Intel Corporation, 2005) using the subroutine ‘four1’ provided by Numerical Recipes in Fortran 77 (see, e.g., Reference 16A). This radix-2 implementation can apply to real data arrays of length 2N although it may not be the most efficient FFT code (see, e.g., Reference 17A). The Fourier power spectrum can be computed, for example, as the magnitude of the real and imaginary parts of the FFT as computed in double-precision mode, and plotted versus frequency.

The ensemble average method of spectral analysis has been described elsewhere (see, e.g., References 6A and 7A). In short, the ensemble average vector e w, for example, can be obtained by averaging successive mean-zero signal segments of length w, for example:

ew=1/n·Uw·b (25a)

Uw=[IwIw . . . Iw] (25b)

where b can be, for example, the signal vector of length N and Iw can be w×w identity submatrices used, for example, to form the signal segments that can be extracted from x and summed. The pixel brightness signals b was not windowed or otherwise filtered prior to analysis using ensemble averaging. The number of signal segments of window length w being summed can be, for example:

n=int(N/w) (26)

where int can typically be needed if n·w≠N. The power in the ensemble average can be given by, for example:

Pw=1/wewT·ew (27)

To generate an ensemble power spectrum, the RMS power can be utilized to reduce the effect of outliers (see, e.g., References 6A and 7A), where, for example:

PwRMS=sqrt(Pw) (28)

where sqrt can be, for example, the square root function and the units can be millivolts. The power spectrum can be then formed by plotting sqrt(n)×PwRMS versus frequency f, where, for example:

f=sample rate/w (29)

The sqrt(n) term levels the noise floor, which can be otherwise diminish by 1/sqrt(n), the falloff per number of summations n used for ensemble averaging. As an additional device to level the noise floor, the linear regression line can be calculated from the graph points and then subtracted from these points. For simplicity, the ensemble average spectrum can be computed using integer values of w, which can result in higher resolution at lower frequencies and lower resolution at higher frequencies due to the relationship of exemplary Eq. (29). If, however, fractional values of w can be used with interpolation between data points, the ensemble average frequency spectrum can be made uniform in resolution.

### Exemplary Computational Considerations

The same FORTRAN compiler that was used for Fourier analysis can also be utilized to calculate or determine the ensemble average spectrum (e.g., single precision mode). Previously, the DP has been observed to occur in the range of about 1-20 seconds (see, e.g., Reference 8A). Thus, for both the Fourier and ensemble averaging methods, the spectral range can be selected as, for example, about 0.05 Hz (e.g., period=20 seconds) to about 1 Hz (e.g., period=1 second). Typical FFT procedures generally require log N·N operations to complete. For the radix-2 implementation, the 200 frames padded to 2n=256 typically need approximately 2.4·256=614 operations to calculate. By comparison, the ensemble procedure can use the number of frames that can be available (e.g., about 200 in this exemplary case). Its spectrum can range from highest frequency (e.g., segment length w=2) to lowest frequency (e.g., w N/2). To detect the DP in the range of about 1-20 s, segment lengths from w=2 (e.g., period 1 s) to 40 (e.g., period 20 s) can be used as endpoints for spectral analysis. For spectral power computation, for example:

$$\begin{matrix}\# operations \approx [ {\sum }_{i} ( ni - 1 ) * wi ] + wi , i = x to y & ( 30 a ) \\ = {\sum }_{i} N , i = x to y & ( 30 b ) \\ = ( y - x ) \cdot N & ( 30 c ) \\ = c \cdot N & ( 30 d )\end{matrix}$$

where the value of n can be obtained from Eq. (26), x and y can be the spectral endpoints, c=y−x can be the number of frequency components computed and/or determined per spectrum, and the +wi term on the RHS in Eq. (30a) can be due to the sum of squares divided by w calculation which determines the ensemble average power Eq. (27). Since the ensemble average for segment length wi=wj/2 can be computed as, for example:

e_{wi}=e_{wj}(1:wⱼ/2)+e_{wj}(wⱼ/2+1:wⱼ) (31)

The number of operations to compute or determine the ensemble spectrum can be readily reduced from c·N (exemplary Eq. (30d)) to c/2·N using exemplary Eq. (31). For x=2 and y=40, c=39, so that for N=200 frames, about 7800 operations can typically be needed to compute an ensemble spectrum, as compared with about 614 for Fourier. However, most of the ensemble average operations can be simple addition. Thus, without testing, it may typically not be apparent whether the Fourier or ensemble average method will be faster to compute the power spectra. Speed can be an important consideration when many video clips from many patients, and/or longer series lengths than about 200, can be used for analysis in future studies. As a test of computational speed, the Fourier versus ensemble spectral calculation over 576×576=331776 pixels can be determined, for example, by using the internal Fortran function ‘etime’ (e.g., user elapsed time), which can be printed on the computer screen during program execution. The difference in the elapsed program run time at start versus end of the 576×576 pixel spectra routine can be taken as the spectral computation time. For faster Fourier computation, the variables can be computed in single-rather than double-precision mode in the speed calculations, which can be observed to reduce computation time by about 10% without evident quantitative effect on Fourier spectral calculation. Speed measurement can be repeated 10 times each for Fourier and ensemble, with pauses of several minutes in between to facilitate the computer to return to its quiescent state.

### Exemplary Results

The exemplary result from a repeating 10 frame sequence is shown, for example, in the exemplary images of FIGS. 12A-12F to highlight how additive noise can affect the images. FIGS. 12A-12F each show the result of ensemble averaging every 10th frame from a 200 frame synthesized series (e.g., frames 1, 11, . . . , 191). Since the repeating sequence can be, for example, 10 frames long, this can result in correlation in the ensemble average. In FIG. 12A, the result is depicted, for example, when the exemplary 200 frame series lacked any additive noise. Thus, the average shown in the exemplary image of FIG. 12A, for example, can be simply the first image frame in the 10 frame sequence that was used for synthesis. The detail in the image of FIG. 12A includes presence of numerous mucosal folds, small mucosal surface abnormalities and extraneous substances. This can be a typical image from celiac video clips taken from the distal duodenum. Subsequently, noise was imparted to the same synthesized 200 frame series. When a random temporal shift of 1-5 frames was imposed on the series, the resulting averaged image from frames 1, 11, . . . , 191 is shown, for example, in FIGS. 12B-12F, respectively. Thus, as shown in the exemplary image of FIG. 12B, there is, for example, a random temporal phase shift of ±1 frame in the 200 frame sequence. The frame shift can be increased in each successive figure, thus in FIG. 12F, there can be a random temporal shift of ±5 frames in the 200 frame sequence. As a result of larger phase shifts, the images in FIGS. 12D-12F can have partially visually morphed to resemble other images in the 10 frame sequence. However, features from the original image having marked contrast as compared to the background can be retained—for example at the asterisks in each frame. Additionally, an imposition of three air bubble frames in the series that was averaged (e.g., 1, 11, . . . , 191) is shown, for example, in FIG. 12F. Air bubbles can be clearly evident within the image, particularly in the lower right quadrant, which contributes to the noise level. Spectral analysis of the series can be expected to readily detect the 10 frame period in the series when no noise was imparted (e.g., FIG. 12A), but to have progressively more difficulty as extraneous features were imposed (e.g., FIGS. 12B-12F).

According to certain further exemplary embodiments of the present disclosure, two additional types of image degradation that can be imposed on the substantially same video clip series as is shown in FIGS. 12A-12F are noted in FIGS. 13A-13F, respectively. For example, FIG. 13A is substantially the same as FIG. 13A, and again is an average of, for example, the 1st 11th, . . . , 191st frames in the synthesized video clip series. Since the synthesized series included, for example, a 10 frame sequence that was repeated, FIG. 13A is also the first frame in that sequence. FIGS. 13B-13F were formed after imposing a maximum spatial frame shift, for example, of 20 pixels on the 200 frame series. As described herein, each row of pixels in the original images used in the series can be rotated by up to about 20 pixels per image. The same procedure and magnitude of spatial frame shift can be imposed in all of the exemplary images of FIGS. 13B-13F, Additionally, as shown in FIGS. 13B-13F, there can be added a successively increased random noise content, for example, with X=10, 50, 100, 150, and about 180 frames at the end of the about 200 frame series being switched to white noise frames, respectively. Thus, FIG. 13B can be constructed with only one noise frame of the 20 frames 1, 11, . . . , 191 used for ensemble averaging, FIG. 13C with 5, FIG. 13D with 10, FIG. 13E with 15, and FIG. 13F with 18 noise frames out of 20 frames. It can be evident that FIG. 37F only slightly resembles the original image depicted.

An example of spectral analysis using the Fourier method is shown, for example, in FIG. 14, again for the case of a 10 frame repetitive sequence for continuity with FIGS. 12A-12F and 13A-13F (e.g., DP=5 s, DF=0.2 Hz). To the 200 frame series, random temporal shift, for example, of up to about +4 frames was imparted and random noise was added. The number of random noise frames out of 200 total frames used for analysis is shown at upper left in each FIGS. 13A-13F. Even in the case of no additive random noise, and only temporal frame shift imposed on the series, the harmonic peaks show a drastic split (e.g., see graph of FIG. 14A). At the about 0.2 Hz mark, there can be a small peak present—the split peaks can be off-center. Furthermore, peaks of greater energy can occur, for example, at about 0.4 Hz and about 0.6 Hz. Thus, this spectrum may not be used to correctly determine the DP. Similar spectral features are present in the exemplary graph of FIG. 14B, when, for example, 50 random white noise frames can be switched into the 200 frame series. In the exemplary graph of FIG. 14C, the increased additive random noise at the level of 100 frames, half the series, can have a smoothing effect on the spectral peaks. Still, the peak near about 0.2 Hz can be substantially off-center, with the peaks at about 0.4 Hz and about 0.6 Hz, for example, being of greater energy. At the highest additive random white noise level (e.g., the exemplary graph of FIG. 14D), all of the harmonics can shift off-center in different directions, they become blunted, and the peak near about 0.2 Hz can be further eroded. Thus, there can be difficulty in using the Fourier method for analysis of this videocapsule frame series for DP determination at the imposed level of noise degradation.

Analysis of the same 200 frame series with the same additive noise and temporal phase shift levels as depicted in FIGS. 14A-14H is shown in the exemplary graphs of FIGS. 15A-15D, when the ensemble average procedure was used, for example, for spectral analysis. In each figure, the dominant peak occurs, for example, at about 0.2 Hz. Subharmonics and the superharmonic at 0.4 Hz can be evident but they can be lesser in value. There can be little difference in the detail in each spectrum, for example, the procedure can be robust to varying, even overwhelming, levels of additive noise. Therefore, in each figure, the DF at about 0.2 Hz (DP of about 5 s), for example, can be correctly identified. Because of the f=1/w relationship (see exemplary Eq. (28)) the frequency resolution may not typically be uniform; however, the detection of DP can be unaffected.

Fourier spectra when all four types of image degradation can be added to the 200 frame series (see above) are shown, for example, in the exemplary graphs of FIGS. 16A-16D. Again for ease of comparison with the other figures, in this exemplary series, a 10 frame repeating sequence can be also used (e.g., DF=0.2 Hz, DP=5 s). The series can have both spatial phase noise (e.g., about ±10 pixels) and temporal phase noise (e.g., about ±3 frames) imparted as well as additive random noise, and eight air bubble frames can be switched in. As shown in the graphs of in FIGS. 14A-14D and 15A-15D, the number of random noise frames is shown, for example, at upper left in each figure. As in FIG. 14A, the harmonic peaks can be split when no random noise is added (see, e.g., FIG. 16A). The peak with greatest energy can occur at, for example, about 0.17 Hz, and the harmonic peaks can be of lesser magnitude. The tallest peak can be maintained at random noise levels of for example, about 50 and about 100 frames (e.g., FIGS. 14B, 15B, 16B and 14C, 15C and 16C). However at the highest additive random noise level shown (e.g., 180 of FIG. 16D) the dominant peak has typically been completely corrupted so that the new dominant peak occurs at, for example, about 0.09 Hz, barely above the noise floor. Thus, in this example as in FIGS. 14A-14D, Fourier spectra may not be accurate for pinpointing the DP.

Spectra created using the exemplary ensemble average method are shown, for example, in the exemplary graphs of FIGS. 17A-17D for the substantially same data as those from which FIGS. 16A-16D were was constructed. As in the graphs of FIGS. 15A-15D ensemble average spectra, the ensemble average spectra of FIGS. 17A-17D, respectively, correctly depict, for example, about 0.2 Hz as the DF. There may be no shift or corruption of the dominant peak, for example, even at the highest additive random noise level of about 180 frames. At the about 180 noise frame level, only about 20 frames can be actual signal (e.g., about 10%), less those frames for which the air bubble frame was switched in. There can be however, a slight broadening of some dominant peaks (e.g., see FIGS. 17B and 17D).

### Exemplary Summary Statistics

As is shown in Table 4 below, for all additive noise levels, the mean absolute difference between estimated versus actual DP can be, for example, about 0.0547±0.0688 Hz for Fourier versus about 0.0031+0.0127 Hz for ensemble (e.g., p<0.001 in mean and standard deviation). The mean time for computing 331,776 pixel spectra per video clip can be, for example, about 12.31±0.01 s for Fourier versus about 4.86±0.01 s for ensemble (e.g., p<0.001).

TABLE 4

| | | | Sig- | Fourier | Ensemble | Sig- |
|---|---|---|---|---|---|---|
| Statistic | Fourier* | Ensemble* | nificance | ^ | ^ | nificance |
| MN | 0.0547 | 0.0031 | p < 0.001 | 12.31 | 4.86 | p < 0.001 |
| SD | 0.0668 | 0.0127 | p < 0.001 | 0.01 | 0.01 | MS |

According to additional exemplary embodiments of the present disclosure, pixel spectral analysis for videocapsule image quantization can be provided. Certain exemplary embodiments can show that even in presence of overwhelming noise and extraneous features imposed upon small intestinal mucosal image series, examples of which are shown in the exemplary images of FIGS. 12A-12F and 13A-13F, the exemplary pixel-by-pixel procedure of frequency analysis can be useful to detect the DP when ensemble averaging can be used for computation. Additionally, the exemplary ensemble average calculation has an advantage of speed over the Fourier analysis using the computer described above. Previously, the average brightness of the entire image frame, for 200 video clip frames, was for example, used as inputs for spectral analysis (see, e.g., Reference 18A), for example:

b=<b1>,<b2>, . . . ,<b200> (32)

where b can be the input for spectral analysis and <•> can denote the frame average brightness for frames 1-200. This simpler method was found useful to find a significant DP difference in celiac versus control video clips (e.g., longer DP in celiacs). Yet, the exemplary pixel-by-pixel spectral calculation, followed by averaging to form the mean spectrum, can potentially be more efficacious for detecting subtle periodicities in videocapsule images because more information can be accounted for.

### Exemplary Analysis of Videocapsule Endoscopy Images

Endoscopy of the small intestine can be helpful for detecting villous atrophy, a common manifestation of untreated celiac disease, although this can typically be confirmed by biopsy (see, e.g., Reference 19A). The typical treatment for celiac disease currently available that can restore the intestinal villi and also eliminate systemic symptoms of the disease, can be a lifelong gluten-free diet (see, e.g., References 9A and 19A). However, months on the diet can typically be needed to substantially restore the small intestinal villi, and in some patients only partial restoration occurs, or there may be no restoration. Among prior quantitative analysis studies of the small intestine to detect villous atrophy, duodenal features have been classified using Fourier filters in magnifying endoscopic images (see, e.g., Reference 20A). Yet, some intestinal regions lack visible change while villous atrophy can be present, which can diminish the sensitivity of the classification method. The textural properties of images from the small intestinal mucosa in celiac disease has been investigated (see, e.g., Reference 18A).

The variance in grayscale brightness can be used as an estimate of texture. Over 200 image frame series in celiac versus control videocapsule studies, the celiacs typically had significantly greater texture magnitude even in distal portions of the small intestine (e.g., jejunum and ileum). This can suggest the possibility that villous atrophy can be widespread in the intestinal lumen in untreated celiac patients, but can be below the threshold for visual detection by eye. Quantitative parameterization over 200 sequential images can therefore be expected to have merit for analysis of small intestinal pathology in these patients. Yet, the textural procedure can be sensitive to ambient conditions including changing camera angle with respect to the luminal wall, and to illumination (see, e.g., References 8A and 18A). Thus, more recently using frequency analysis over 200 frames would be anticipated to be sensitive to periodic oscillations in frame-to-frame brightness variation due to small intestinal motility. It can be possible that the exemplary method can be robust to ambient conditions like camera angle and illumination, as the oscillations can be typically reflected in the frequency content while changes in ambient conditions can mostly just affect the overall spectral power (see, e.g., Reference 8A). Exemplary embodiments of the present disclosure can provide evidence that the DP can be in fact an important repeating pattern in 200 frame series, where importance can be synonymous with having the greatest spectral power, and that pixel-by-pixel spectra calculation can be robust to even large-scale extraneous features.

Although descriptions of certain exemplary embodiments of the present disclosure have been limited, for simplicity, to converting the color videocapsule images to 256 level grayscale for quantitative analysis, abnormal patterns can also be detected in color space using nonlinear methods (see, e.g., Reference 21A). Here, the nonlinear approach was used to detect specific features—in this case ulcerous regions versus normal mucous membrane in the small intestine. Their analysis showed that the green component of RGB can contain the bulk of the ulcer information, with classification accuracy exceeding about 95.5%. Although small intestine villi can be much more subtle in structure than are ulcerous regions, the use of a specific color (e.g., green, red or blue) rather than grayscale can be useful to improve the exemplary procedures for frequency detection.

### Exemplary Motility Measurement in Videocapsule Endoscopy

Although videocapsule endoscopy has been commercially available for approximately 10 years (see, e.g., Reference 22A), the images are presently used by the gastroenterologist typically as a qualitative assist device when assessing the extent and severity of villous atrophy (see, e.g., References 23A-25A). Gastrointestinal motility can also likely be altered in untreated celiac disease due to injury to the mucosa, but can typically only be indirectly gauged by measuring the transit time from proximal to distal small intestine. To establish a more direct link between the mechanical characteristics of the small intestine and celiac disease, the exemplary frame-by-frame frequency analysis has been proposed, and in a prior study found a direct correlation between transit time and DP (see, e.g., Reference 8A).

FIG. 18 shows a block diagram of an exemplary embodiment of a system according to the present disclosure. For example, exemplary procedures in accordance with the present disclosure described herein can be performed by a processing arrangement and/or a processing arrangement 102. Such processing/computing arrangement 102 can be, for example entirely or a part of, or include, but not limited to, a computer/processor 104 that can include, for example one or more microprocessors, and use instructions stored on a computer-accessible medium (e.g., RAM, ROM, hard drive, or other storage device).

As shown in FIG. 18, for example a computer-accessible medium 106 (e.g., as described herein above, a storage device such as a hard disk, floppy disk, memory stick, CD-ROM, RAM, ROM, etc., or a collection thereof) can be provided (e.g., in communication with the processing arrangement 102), according to an exemplary embodiment of the present disclosure. The computer-accessible medium 106 can contain executable instructions 108 thereon. In addition or alternatively, a storage arrangement 110 can be provided separately from the computer-accessible medium 106, which can provide the instructions to the processing arrangement 102 so as to configure the processing arrangement to execute various exemplary procedures, processes and methods, as described herein, for example.

Further, the exemplary processing arrangement 102 can be provided with or include an input/output arrangement 114, which can include, for example a wired network, a wireless network, the internet, an intranet, a data collection probe, a sensor, etc. As shown in FIG. 18, the exemplary processing arrangement 102 can be in communication with an exemplary display arrangement 112, which, according to certain exemplary embodiments of the present disclosure, can be a touch-screen configured for inputting information to the processing arrangement in addition to outputting information from the processing arrangement, for example. Further, the exemplary display 112 and/or a storage arrangement 110 can be used to display and/or store data in a user-accessible format and/or user-readable format.

### Exemplary Clinical Data Acquisition

In one example, atrial electrograms was recorded in 19 patients referred to the Columbia University Medical Center cardiac electrophysiology laboratory for catheter ablation of AF. Nine patients had clinical paroxysmal AF with normal sinus rhythm as their baseline cardiac rhythm. AF was induced by burst pacing from the coronary sinus or from the right atrial lateral wall, and continued for at least 10 minutes prior to data collection. Ten other patients had longstanding persistent AF without interruption for several months to many years prior to catheter mapping and ablation. Bipolar atrial mapping was performed using a NaviStar ThermoCool catheter, 7.5 F, 3.5 mm tip, with about 2 mm spacing between bipoles (e.g., Biosense-Webster Inc., Diamond Bar, Calif., USA). Electrograms were acquired using the General Electric CardioLab system (e.g., GE Healthcare, Waukesha, Wis.), and filtered at acquisition from about 30-500 Hz with a single pole bandpass filter to remove baseline drift and high frequency noise. The filtered signals were sampled at about 977 Hz and stored. Although the bandpass high end was slightly above the Nyquist frequency, negligible signal energy can reside in this range. (See, e.g., Reference 50). For example, only signals identified as CFAE by two cardiac electrophysiologists were included for retrospective analysis. CFAE recordings were obtained from two sites outside the ostia of each of the four pulmonary veins. Recordings were obtained at two left atrial free wall sites, one in the mid-posterior wall, and another on the anterior ridge at the base of the left atrial appendage.

### Exemplary CFAE Data Structure

For example, a total of about 204 recording sequences of length greater than about 16 s, acquired from both paroxysmal and longstanding persistent AF patients, and all meeting the criteria for CFAE, were selected for analysis. DFT and NSE power spectra can be computed in the standard electrophysiologic frequency range from 3-12 Hz. The time windows over which spectra can be calculated can be, for example, about 8192, 4096, 2048, 1024 and 512 sample points (e.g., about 8 s, 4 s, 2 s, 1 s and 0.5 s). Binary step changes in window length were used so as to be maximally compatible with the DFT method. The upper limit of, for example, about 8192 points was considered the optimal time window. (See, e.g., Reference 49). The lower limit of 512 sample points was the theoretical minimum to analyze 3 Hz content, which has a period of 977 samples per second/3 per second=325 sample points for this data. The next binary step at 256 sample points may not extend the entire period of 3 Hz frequency content. Rectangular windowing can be used to extract segments for analysis, as unlike other window functions, it may not diminish frequency resolution. (See, e.g., Reference 51). For the DFT calculation, at the about 4096, 2048, 1024, and 512 sample point analysis windows were padded with zeros to 8192 points. For conformity, all DFT and NSE analyses were done using the same 8192 sample point intervals of data. Thus, at the 4096 time resolution level, spectra was generated for two successive 4096 point windows and then averaged, and similarly four 2048 point windows, eight 1024 point windows and sixteen 512 point windows were averaged for the 2048, 1024, and 512 time resolution levels, respectively.

### Exemplary Digital Power Spectra

The DFT power spectrum can be constructed using a radix-2 implementation. (See, e.g., Reference 52). The NSE power spectrum can be constructed as follows. (See, e.g., Reference 50). In the equations, an underscore can denote a vector, a capital letter can signify a matrix, and the first subscript can give the dimensionality of the vector or matrix. A vector e_{w} of dimension w×1 can be calculated by averaging n successive segments of an N×1 dimensional signal x_{N}, where x_{N} can be a CFAE signal normalized to mean zero and unity variance prior to analysis. Each segment x_{w,i} of this signal, of dimension w×1, can be used for averaging, for example:

$${\underline{e}}_{w} = \frac{1}{n} {\sum }_{i} {\underline{x}}_{w , i} , i = 1 to n \qquad ( 33 )$$

where, for example:

$${\underline{x}}_{N} = [ \begin{matrix}{\underline{x}}_{w , i} \\ {\underline{x}}_{w , 2} \\ … \\ {\underline{x}}_{w , n}\end{matrix} ] \qquad ( 34 )$$

The exemplary process described by Eqs. (33)-(34) is illustrated in an exemplary graph and flow diagram of FIG. 26. For example, a selected CFAE, signal x, can be graphed from discrete sample point 1 to 1000. For example, let w=250 sample points. Segments i=1 to 4 can be noted below x, and they can be the signal segments x_{w,i} for w=250. When the four segments shown can be averaged together, the result can be depicted at the bottom of FIG. 26. Any periodicity at w=250 can be reinforced in the sum, while random components can diminish. Even in the presence of phase jitter, quasi-periodic components can be reinforced. (See, e.g., Reference 53). For a signal x_{N} of length N, the total number of signal segments, and therefore the total number of summations used for ensemble averaging, can be given by, for example:

$$n = int \frac{N}{w} \qquad ( 35 )$$

with ‘int’ being the integer function, and the real number being rounded down. From Eqs. (33)-(35), the ensemble average for any segment length w can be written in compact form:

$${\underline{e}}_{w} = \frac{1}{n} \cdot {U}_{w \times N} \cdot {\underline{x}}_{N} \qquad ( 36 )$$

where, for example:

U_{w×N}=[I_{w×w}I_{w×w} . . . I_{w×w}] (37)

with U_{w×N} being a w×N dimensional summing matrix and I_{w×w} can be w×w dimensional identity submatrices used to extract the signal segments from x_{N}. Identity matrices can be sparse, and the total number of nonzero summations from Eqs. (36) and (37) can be n, not N, as in Eq. (33); hence the scale term can be 1/n in this equation. From Eq. (35), if N/w may not be an integer, then the right edge of U_{w×N} can be padded with 0's.

The relationship between segment length w used for averaging, which can be a period, and frequency f can be given by, for example:

$$f = \frac{sample rate}{w} \qquad ( 38 )$$

For any particular segment length w, the power in the ensemble average can be, for example:

$$\begin{matrix}{P}_{w} = \frac{1}{w} \cdot {\underline{e}}_{w}^{T} \cdot {\underline{e}}_{w} & ( 39 a ) \\ = \frac{1}{{n}^{2} w} {\sum }_{i} {\sum }_{j} {\underline{x}}_{w , i}^{T} \cdot {\underline{x}}_{w , j} i = 1 to n , j = 1 to n & ( 39 b ) \\ = \frac{1}{nN} \cdot {\underline{x}}_{N}^{T} \cdot {U}_{N \times w} \cdot {U}_{w \times N} \cdot {\underline{x}}_{N} & ( 39 c )\end{matrix}$$

for signal segments x_{w,i} and x_{w,j}, where the transpose of the summing matrix can be given by, for example:

U_{w×N}^{T}=U_{N×w} (40)

Eq. (39a) can be based upon the definition of power—it can be the sum of squares of each element of e_{w} divided by the total number of such summations w. Eq. (39b) can result from substituting Eq. (33) into Eq. (39a), and Eq. (39c) can result from substituting Eq. (36) into Eq. (39a). Eq. (39b) can be similar to computing the average of the estimated autocorrelation function for all lags 1w, 2w, . . . nw, which can be given by, for example:

$$\begin{matrix}rav ( w ) = \frac{1}{nN} {\sum }_{k} {\underline{X}}_{N}^{T} * {\underline{X}}_{N} , φ = k * w & k = 1 to n & ( 41 a ) \\ = \frac{1}{{n}^{2} W} {\sum }_{k} {\sum }_{i} {\underline{X}}_{W}^{T} , i {\underline{X}}_{W} , i + k & i = 1 to n , k = 1 to n & ( 41 b )\end{matrix}$$

where x_{N,φ=k·w} can be shifted in phase from x_{N} by φ=k·w and Eq. (41b) can be computed over an interval 2N. An example CFAE is shown in FIG. 27A, and lags in its autocorrelation function are shown in FIG. 27B when using w=125 sample points f=7.8 Hz. The value of the autocorrelation function at all of the lags at 1w, 2w, nw can be averaged to form rav(w) in Eq. 41.

Short segments x_{w,i} in Eq. (41b) can be considered as a first approximation to be mean zero and unity variance, so that the autocorrelation and autocovariance functions can be considered to be equivalent and can be used interchangeably. To implement Eq. (41a) in computer software, the following line of software code can be used, for example:

Rac(w)=rav(w)+x(i)*x(i+kw) i=1 to n,k=1 to n (42)

where x(i) can be a discrete sample point, and x(i+kw) can be a sample point shifted by i+kw for lags 1w, 2w, . . . , nw. This spectral estimator can then be plotted as rav(w)/N versus the frequency f=sample rate/w. The mean squared error function can be equivalent to the autocorrelation function as a spectral estimator.

In the above derivation, the NSE power spectrum can be formed by modeling the signal autocorrelation function. Like the NSE estimator, the DFT power spectrum can also be formed by modeling the signal autocorrelation function. Based on the Wiener-Khinchin theorem, the power spectrum of signal x_{N} can be given by the Fourier transform of its autocorrelation function, which can be, for example:

$$\begin{matrix}S ( f ) = \frac{1}{N} {\sum }_{φ} ( {\underline{X}}_{N} * {\underline{X}}_{N , i + 1} ) {e}^{- 2 π if φ} & ( 43 a ) \\ = \frac{1}{nw} {\sum }_{i} {\sum }_{w} ( {\underline{X}}_{w , i}^{T} {X}_{w , i + 1} ) {e}^{- 2 π ifw} & ( 43 b ) \\ & ( 43 )\end{matrix}$$

where S can be the power spectral density, x_{N}·x_{N,φ} can be the autocorrelation function with lag φ, and Eq. (43b) can be similar to Eq. (41) for one lag (k=1), with lag symbol φ being replaced by w, and with nw=N. The DFT power spectral density calculation can thus model the autocorrelation function by sinusoidal decomposition.

While the DFT can incorporate a general basis that can be sinusoidal, the NSE basis can be data-driven. To show this, signal x_{N}N x can be projected into NSE space using the following N×N transformation matrix (see, e.g., Reference 50) where, for example:

$${T}_{N \times N} ( w ) = {U}_{N \times w} \cdot {U}_{w \times N} = [ \begin{matrix}{I}_{w} & {I}_{w} & … & {I}_{w} \\ {I}_{w} & {I}_{w} & … & {I}_{w} \\ … & & & \\ {I}_{w} & {I}_{w} & … & {I}_{w}\end{matrix} ] \qquad ( 44 )$$

Signal x_{N} can then be decomposed using the linear transformation where, for example:

$${\underline{a}}_{N} ( w ) = \frac{1}{n} {T}_{N \times N} ( w ) \cdot {\underline{x}}_{N} \qquad ( 45 )$$

where a_{N}(w) can be a set of basis vectors of dimension N×1. The orthogonality of any two basis vectors with periods w=y and w=z can be given by, for example:

$$\frac{{\underline{a}}_{N}^{T} ( y ) \cdot {\underline{a}}_{N} ( z )}{\sqrt{[ {\underline{a}}_{N}^{T} ( y ) \cdot {\underline{a}}_{N} ( y ) ] [ ( {\underline{a}}_{N}^{T} ( z ) \cdot {\underline{a}}_{N} ( z ) ]}} = cos θ \qquad ( 46 )$$

where from Eq. (45), the numerator in Eq. (46) can be rewritten as, for example:

$${\underline{a}}_{N}^{T} ( y ) \cdot {\underline{a}}_{N} ( z ) = \frac{1}{n ( y ) n ( z )} {\underline{x}}_{N}^{T} \cdot {T}_{N \times N} ( y ) \cdot {T}_{N \times N} ( z ) \cdot {\underline{x}}_{N} \qquad ( 47 )$$

and n(y) and n(z) can be values of n (e.g., Eq. (35)) for w=y and w=z. As the angle θ→90° (e.g., left-hand-side in Eq. (46)→0) it can be indicative of more nearly orthogonal vectors. Orthogonality can be exact when a_{N}^{T}(y)·a_{N}(z)=0 (e.g., Eq. (46)), or equivalently when the inner product of each row in T_{N×N}(y) with the corresponding column in T_{N×N}(z) equals zero (Eq. 47). Orthogonality can be approximate when y and z can have a distant integer relationship over N, so that a_{N}^{T}(y)·a_{N}(z) in Eq. (46), and the inner products of corresponding rows and columns of T_{N×N}(y) and T_{N×N}(Z) in Eq. (47), can be small but nonzero.

The transformation matrix T_{N×N}(W) in Eqs. (44) and (45) can act to decompose the signal into periodic ensemble averages. An example is shown in FIGS. 28A-28D. The CFAE can be from the posterior left atrial free wall in a persistent AF patient (e.g., FIG. 28A). The NSE spectrum is shown in the exemplary graph of FIG. 28B. The DF, which can be the tallest fundamental spectral peak in the range of interest (see, e.g., References 54 and 55), occurs at and 7.08 Hz (e.g., w=138 for 977 Hz sampling rate), noted by *. A minimum point at and 7.29 Hz (e.g., w=134) can be noted by **. The basis vector a_{N}(w) from Eq. (45), consisting of repeated ensemble averages, is shown in FIG. 28C for the DF, while for the minimum point at ** it is shown in the exemplary graph of FIG. 28D to the same scale. There can be substantial power in the basis vector of FIG. 28C, because it can align with CFAE deflections (e.g., FIG. 28A), while there can be much less power in the basis vector of FIG. 28D.

### Exemplary NSE Frequency Resolution

An exemplary frequency resolution of the NSE for any particular segment length w=k, where k can be an integer, can be described as, for example:

$$fr ( k ) = \frac{rate}{k} - \frac{rate}{k + 1} \qquad ( 48 )$$

Eq. (48) can be rewritten as, for example:

$$fr ( k ) = rate \cdot ( \frac{1}{k} - \frac{1}{k + 1} ) \\ = rate \cdot ( \frac{1}{{k}^{2} + k} ) \qquad ( 49 )$$

For w=k large:

$$For w = k large ; \\ fr ( w ) \approx \frac{rate}{{w}^{2}} \qquad ( 50 )$$

Thus, the NSE frequency resolution can be proportional to rate/period². It can improve as the period w=k can increase (e.g., smaller value of fr(w)), for example, at lower frequency values. The NSE estimator can contain a maximum of N/2 spectral points (e.g., an average can contain at least two segments), the same as for the DFT. Therefore the NSE and DFT estimators can have equal frequency resolution overall. Although time duration does not directly affect the NSE frequency resolution (e.g., Eq. (50)) it can indirectly affect resolution, because as time duration diminishes, the number of signal segments n from Eq. (35) used to form the ensemble average estimate decreases. The cruder estimate can be anticipated to somewhat diminish accuracy.

### Exemplary Improved NSE Time Resolution

For example, by forming the ensemble average estimate from longer intervals, and then projecting the estimate onto shorter data intervals, the NSE time resolution could be extended. From Eq. (39), the approximate power over a time duration consisting of a reduced number of signal segments l<n can be given by, for example:

$$〈 {P}_{w} 〉 = \frac{1}{w \cdot 𝓁} {\sum }_{i} ( {\underline{e}}_{w}^{T} \cdot {\underline{x}}_{w , t} ) , i = 1 to 𝓁 < n \\ = \frac{1}{w \cdot 𝓁} {\underline{e}}_{w}^{T} \cdot {\sum }_{i} {\underline{x}}_{w , i} , i = 1 to 𝓁 < n \qquad ( 51 )$$

Using Eq. (51), the local frequency content, which can be estimated from the average computed over l segments, can be compared to the global frequency content, for example, the ensemble average e_{w} computed over n segments. In the exemplary study, using ensemble averages computed from about 2048 points, power spectra can be estimated for l=1024 and l=512 points using Eq. (51).

Exemplary Comparison of Estimators using Repeating Electrogram Patterns

For comparison of NSE versus DFT spectral estimators, an exemplary repetitive electrogram pattern can be constructed. The pattern can be extracted from a CFAE at a random point and with random window size, and adjusted to mean zero and a standard deviation of about 0.08, which can be on the order of 2× the average standard deviation of the CFAE signals acquired for the exemplary study prior to their normalization. The pattern can then be repeated to a total length of N=8192 discrete sample points. The about 204 CFAE themselves can be used as interference having unknown frequency content, by adding the repeating electrogram pattern to each CFAE. It can be determined whether the frequency of the repeating electrogram pattern could be detected as the DF in the power spectrum of the resulting signal. Jitter can also be introduced by randomly shifting each repeating electrogram pattern by up to about ±5 sample points (e.g., approximately ±5 ms) to simulate phase noise. The DF can be measured for 20 different electrogram patterns with phase noise using the DFT and NSE spectral estimators. Estimates can be considered satisfactory when the absolute error can be less than about 0.5 Hz.

Examples of a repeating electrogram pattern added to a CFAE are shown in the exemplary graphs of FIGS. 29A and 29B. For example, FIG. 29A illustrates a graph providing sample points 1-500 of a CFAE from the left superior pulmonary vein ostium in a persistent AF patient (e.g., element 2910). Overlapping it can be the same CFAE with a repeating electrogram pattern added; having a period of approximately 170 sample points or about 5.75 Hz in frequency (e.g., element 2905). The cycles of repeating pattern are labeled from a-d at the large downward deflection, which can be a prominent fiduciary point. These downward deflections can change from one cycle to the next due to the level of interference from the added CFAE. The horizontal arrows show equal intervals along the traces. The repeating pattern can be shifted by random jitter in segment b-c versus segment c-d, so that the periods between b-c and c-d can be unequal. The cycle length of b-c can be longer than c-d.

In the exemplary graph of FIG. 29B, a CFAE from the left superior pulmonary vein ostium in a paroxysmal AF patient can be graphed from sample points 1-1000. Overlapping it (e.g., element 2915) can be a repeating electrogram pattern, this time having a period of approximately 250 sample points or 4 Hz in frequency, with the CFAE acting as interference. Again, as also shown in FIG. 29A, cycles a-d can be unequal in length due to the phase jitter introduced to the repeating electrogram pattern. For 20 trials, the error can be calculated as the absolute difference in the DF measured from the power spectrum, versus the actual frequency of the repeating electrogram pattern. Significant differences in mean error values for DFT versus NSE measurements can be determined using the paired t-test (e.g., SigmaPlot 2004 for Windows Ver. 9.01, Systat Software, Chicago) at the p<0.05 level.

### Exemplary Real Data Comparison of the Spectral Estimators

Three exemplary spectral properties can be measured from the real data to compare the DFT versus NSE spectral estimators. (See, e.g., Reference 43). The DF, which can be reflective of the atrial activation rate (see, e.g., References 47 and 48), can be determined in the physiologic range of interest, about 3-12 Hz. (See, e.g., Reference 53). The second spectral property that can be measured can be the dominant amplitude (“DA”), defined as the amplitude of the dominant spectral peak. (See, e.g., Reference 43). It can be proportional to the power contained in the fundamental frequency component of the signal, and, therefore, to the proportion of tissue undergoing electrical activation at the cycle length given by the DF. The third measurement, the mean spectral magnitude (“MP”), can reflect the characteristics of all frequency components rather than just the dominant frequency. (See, e.g., Reference 43). The MP can be related to the noise floor, which itself can be dependent upon the degree of randomness in the electrical activation pattern. Measurements can be made at time resolutions of 8 s, 4 s, 2 s, 1 s and 0.5 s.

The DA, DF and MP can be measured and compared for paroxysmal versus persistent CFAE recordings. In accord with prior analyses (see, e.g., Reference 43), for the MP measurement, recordings from all locations can be compared (e.g., 114 persistent and 90 paroxysmal CFAE). Also in accord with prior analyses (see, e.g., Reference 43), for the DA and DF measurements, only recordings from the pulmonary vein ostia can be compared (e.g., 76 persistent and 60 paroxysmal CFAE recordings). The DF can be detected automatically in computer software as the tallest spectral peak in the range of about 3-12 Hz, excluding harmonics. The unpaired t-test can be used to compare the means of paroxysmal versus persistent AF data (e.g., MedCalc ver. 9.5, 2008, MedCalc Software bvba, Mariakerke, Belgium), with the p<0.05 level indicating significance.

### Exemplary Synthetic Data Comparison of the Spectral Estimators

As an additional test of the performance of the NSE versus DFT estimators, a synthetic fractionated electrogram can be constructed and analyzed. It can consist of three additive components, simple period geometrical shapes, with frequencies of about 3.26 Hz, about 4.77 Hz and about 6.98 Hz. Random noise with a standard deviation of about 2.5 millivolts, approximately 50× the standard deviation of the CFAE, can be added to the synthetic fractionated electrogram. It can then be determined whether or not the three largest peaks in the NSE and DFT spectra in the range of about 3-12 Hz, excluding harmonics, can coincide with the frequencies of the additive synthetic components. This can be repeated for 15 trials with a different random noise used on each trial.

### Exemplary Results

In Table 5 below, the average estimation error for detecting the repeating electrogram pattern over 20 trials is shown for DFT versus NSE spectral estimators. The absolute values are given in Hertz. At all levels from 8192 through 512 sample points of time resolution, the NSE estimator can be more accurate than DFT. Thus, for the five resolution levels of about 8 s, about 4 s, about 2 s, about 1 s, about 0.5 s, the error in detecting repeating electrogram patterns can be significantly less when using the NSE estimator as compared with DFT (e.g., p<0.001).

Tables 6-8 herein provide exemplary results for detecting differences in power spectral parameters for paroxysmal versus persistent AF. In Table 6, mean values of the DA parameter are shown. At all time resolutions when using the NSE spectrum for calculation, the DA can be greater in persistent AF (e.g., p<0.0001), indicating that it can often be more predominant as compared with other spectral components in the persistent AF spectra, versus paroxysmal AF spectra where the DF can be less dominant. In Table 7, the mean DF can be higher in persistent as compared with paroxysmal AF for all data. The significance level can be higher for NSE at the 8192, 1024, and 512 levels and can be similar in NSE and DFT at the 4096 and 2048 levels. In Table 8, the mean MP can be larger in paroxysmal as compared with persistent AF for all data. There can be a greater significant difference for the NSE method at the 2048, 1024, and 512 levels. The DFT and NSE estimators have similar significant differences at the 8192 and 4096 levels (e.g., p<0.0001). The larger DA, higher DF, and lower MP in persistent as compared with paroxysmal AF data can be in accord with the known properties of both types of AF, for example, persistent AF activation patterns tend to be more regular and stable, and have a faster rate as compared with paroxysmal AF activation patterns. (See, e.g., References 41, 42 and 53).

In the exemplary graph of FIG. 30A, the synthetic geometric shapes used to test the NSE and DFT estimators are shown. At the top of FIG. 30A, the individual shapes are shown as offset. At the bottom of FIG. 30A, the combined synthetic pattern is shown. In FIG. 30B, the NSE and DFT spectra for the noiseless synthetic fractionated electrogram are shown. For Reference, the frequencies of the individual components are shown as vertical lines 3005. The highest spectral peaks can coincide with the actual synthetic component frequencies for both estimators. For both estimators, there can also be a tall harmonic peak—the second harmonic of the 3.26 Hz components, which can be labeled. For the DFT estimator, the 3.26 Hz and 4.77 Hz frequency peaks are slightly misaligned while for the NSE estimator, the 6.98 Hz peak is slightly misaligned. Overall, the top three spectral peaks in the range 3-12 Hz, excluding harmonics, can coincide with the three synthetic components in 14/15 trials for the NSE estimator, and for 9/15 trials for the DFT estimator. An example is shown in the exemplary graphs of FIGS. 31A and 31B. The top three peaks 3105, excluding harmonics, can coincide with the synthetic component frequencies for the NSE spectrum (e.g., FIG. 31A). Only two of the top three peaks excluding harmonics can coincide with the synthetic component frequencies for the DFT spectrum (e.g., FIG. 31B), where the actual frequencies of the synthetic components are denoted with vertical lines for Reference.

### Exemplary Discussion

In the exemplary study, details concerning an exemplary spectral estimator, or NSE, can be described. The NSE and DFT estimators can be compared to analyze fractionated atrial electrograms acquired from paroxysmal and persistent AF patients. To form the power spectrum, the NSE can average the autocorrelation function at lags, while the DFT can use a sinusoidal approximation to model the autocorrelation function. Differences in modeling the autocorrelation function for power spectrum formation contribute to the differing properties of the DFT and NSE estimators. In contrast to the DFT frequency resolution, which can be proportional to rate/time duration, the exemplary NSE frequency resolution can be proportional to rate/period2. Power spectral equations similar to that of the NSE can be derived from the average autocorrelation and mean squared error functions

The exemplary NSE time resolution at, for example, 1024 and 512 sample points (e.g., about 1 s and about 0.5 s, respectively) can be improved using a temporally globalized ensemble average model over 2 s, which can be projected onto temporally localized data (e.g., Eq. (51)). The global model can contain local information, which can become evident by projection onto the shorter electrogram interval containing localized data. The maximum error in detecting a repeating electrogram pattern can be found to be about 0.896±0.736 Hz for DFT versus about 0.191±0.223 Hz for NSE, which can occur for 0.5 s time windows (e.g., p<0.001; Table 5). The NSE can have significantly improved spectral qualities compared with the DFT across the range of time resolutions used for analysis, from about 8 s to the theoretical minimum time interval for analysis of about 0.5 s (e.g., Table 5). The NSE can also be more useful to determine significant differences in paroxysmal versus persistent CFAE spectral parameters. The NSE spectra can provide the best discrimination of the DA spectral parameter in paroxysmal versus persistent AF as compared with the DFT spectra at all-time resolution levels of about 8 s, 4 s, 2 s, 1 s, 0.5 s (e.g., p<0.0001). NSE spectra provided the best discrimination of DF and MP spectral parameters at three of five time resolution levels. In previous work, the DA and MP spectral parameters have been shown to be correlated to the duration of AF in months, and to the left atrial volume of AF patients. (See, e.g., Reference 42).

The DF spectral parameter can also be very useful for AF patient evaluation in the electrophysiology laboratory. For example, local reentrant circuits can be indicated by lower DFs that coexist in chaotic AF sequences. (See, e.g., Reference 56). Paroxysmal AF, but not persistent AF, can be driven by high DF sources and a left-to-right DF gradient. (See, e.g., Reference 57). A significant reduction in DF in both left and right atria, with a loss of the left-to-right atrial gradient after ablation, can be associated with a higher probability of maintaining sinus rhythm in both paroxysmal and persistent AF patients. (See, e.g., Reference 58). It can also be possible to classify paroxysmal as compared with persistent AF by detecting subtle changes in the DF, combined with analysis of an entropy measure. (See, e.g., Reference 59). Moreover, there can be significant regional variation in the DF in paroxysmal but not persistent AF. (See, e.g., Reference 42).

Although recording intervals of {tilde under (>)}2 s can be utilized for reliable DF measurement using the DFT, as has been shown in the present study (e.g., Table 5) and elsewhere, spectral changes preceding major arrhythmic events such as spontaneous termination of paroxysmal atrial fibrillation can occur over intervals shorter than 2 s. The NSE, but not the DFT, can therefore be suited to this purpose, since the time resolution can be satisfactory down to the theoretical limit of about 0.5 s for the physiologic frequency range of interest (e.g., Table 5). Moreover, subtle spatial gradients in DF of a few tenths of Hertz exist away from the pulmonary veins, and subtle changes in DF of a few tenths of Hertz caused by pharmacologic agents can also occur. These changes may not be readily measurable in patients using the DFT, which can have an error <0.5 Hz only for window segments of about 2 s and greater (e.g., Table 5). Conflicting results from DFT spectral analysis of fractionated atrial electrograms can be partially explained by the lack of time and frequency resolution. The NSE can therefore be helpful to clarify previous findings.

Since wavelet decomposition may not be as commonly used for analysis of AF electrograms as compared with the DFT, and as it estimates different spectral properties, it may not be used for comparison in the exemplary study. However, wavelet decomposition can be useful for applications including the automatic detection of local activation times when the pattern of atrial fibrillation can be complex, for automated description of fractionation morphology in atrial electrograms, extraction of the spatiotemporal characteristics in paroxysmal AF to identify arrhythmogenic regions for catheter ablation, and to predict the spontaneous termination of paroxysmal AF and the outcome of electrical cardioversion in persistent AF patients. Thus the exemplary spectral estimator can provide complimentary information to the DFT and NSE estimators when AF data can be analyzed.

In addition to the application to fractionated atrial electrograms, the exemplary NSE procedure can be implemented for other types of data including the study of ventricular tachyarrhythmia onset and videocapsule image analysis that can be used for screening in celiac disease. In recent investigations, the spectral parameters described in the exemplary study can be used for QRST cancellation, and the exemplary NSE procedure can be implemented for heart sounds quantification. Similar to the exemplary NSE, in a prior exemplary study, heart sounds patterns have been detected by averaging segments of the acoustic signal at different lengths w. Based on these investigations, the exemplary NSE procedure can be generalizable to many types of biomedical data.

The exemplary NSE spectrum can contain subharmonics and cross-terms. (See, e.g., Reference 43). Such components can interfere with DF detection, and can cause the MP parameter to be increased. Second harmonics can be reduced in the exemplary NSE by imparting antisymmetry to the ensemble averages (see, e.g., Reference 43), but this can diminish the power of pertinent frequency components as well. To further reduce subharmonics and cross-terms, higher-order harmonics can be cancelled. Although as shown in the exemplary study where the exemplary NSE procedure can account for inexact periodicity (e.g., phase noise), other procedures to measure frequency content under such conditions can also be helpful to analyze fractionated atrial electrograms. In paroxysmal AF patients, the DF can be related to the degree of fractionation. Therefore the DA and MP spectral parameters can be in part dependent on the DF.

### Exemplary Further Conclusions

In the presence of interference and phase noise, for example, a repeating electrogram pattern can be found to be accurately detected to the theoretical minimum time resolution of about 0.5 s using the exemplary NSE estimator. At all time resolution levels, the exemplary NSE procedure can have negligible bias and significantly reduced variance as compared with the DFT estimator (e.g., Table 5). The exemplary NSE procedure can also be found useful to determine significant differences in the DA, DF and MP spectral parameters in paroxysmal versus persistent CFAE data. Based on both the reduced estimation error in detecting a repeating pattern, and the greater significant differences in real paroxysmal versus persistent AF spectral parameters, the exemplary NSE estimator can be useful for frequency analysis of atrial signals as a comparative technique with respect to the traditional DFT procedure, and to validate the results of the DFT. The NSE can even be useful to provide improved frequency analysis of CFAE data at short time resolutions.

The findings of exemplary study suggest that the exemplary NSE method can provide improved time resolution, which along with the better frequency resolution (see, e.g., Reference 51), can result in more accurate measurement of spectral properties in fractionated atrial electrogram recordings. At the 0.5 s time resolution level, the error can still be below about 0.5 Hz for the exemplary NSE estimator (e.g., Table 5). Regardless of time window, the frequency resolution of the exemplary NSE averages about 0.05 Hz in the 3-12 Hz physiologic frequency band. (See, e.g., Reference 51). This compares with a best time resolution of 2 s for DFT found in this study (e.g., Table 5) and elsewhere, which at a sampling rate of about 1 kHz can correspond to an about 0.5 Hz frequency resolution. As the exemplary NSE technique can be automated without the need for manual correction, user bias can be eliminated, with no need for ad hoc parameterization and input of a priori information, so that it can potentially be applicable to real-time analysis in the clinical electrophysiology laboratory for evaluation of AF patients.

### Exemplary NSE Method

### Exemplary Further Clinical Data Acquisition and Electrophysiologic Mapping

Atrial electrograms have been recorded from 19 patients referred to the Columbia University Medical Center cardiac electrophysiology laboratory for catheter ablation of AF. Nine patients had documented clinical paroxysmal AF. Normal sinus rhythm can be their baseline cardiac rhythm in the electrophysiology laboratory. Induction of AF was done via burst atrial pacing from the coronary sinus or right atrial lateral wall, and allowed to persist for at least 10 minutes prior to signal acquisition. Ten other patients had longstanding persistent AF without interruption for several months to many years prior to catheter mapping and ablation. The bipolar atrial mapping procedure was performed with a NaviStar ThermoCool catheter, 7.5 F, 3.5 mm tip, with 2 mm spacing between bipoles (e.g., Biosense-Webster Inc., Diamond Bar, Calif., USA). The electrogram signals was acquired using a General Electric CardioLab system (e.g., GE Healthcare, Waukesha, Wis.), and filtered at acquisition from approximately 30 to approximately 500 Hz with a single-pole bandpass filter to remove baseline drift and high frequency noise. The filtered signals were digitally sampled at approximately 977 Hz and the digital data can then be stored. Although the high end of the bandpass filter can be slightly above the Nyquist frequency, negligible CFAE signal energy can reside in this frequency range. (See, e.g., Reference 53).

CFAE was identified by two clinical cardiac electrophysiologists. (See, e.g., Reference 44). CFAE recordings of at least 16 seconds in duration were obtained from two sites each outside the ostia of the four pulmonary veins. Similar recordings can be obtained at two left atrial free wall sites, one in the mid posterior wall, and another on the anterior ridge at the base of the left atrial appendage. The mapping catheter was navigated in these pre-specified areas until a CFAE site was identified. A total of 204 CFAE sequences, approximately 90 from paroxysmal and approximately 114 from longstanding AF patients was included in the following quantitative analysis. As in previous studies, to standardize the morphological characteristics, all CFAE was normalized to mean zero and unity variance (e.g., average level=approximately 0 volts, standard deviation and variance=approximately 1). (See, e.g., References 50 and 54).

### Exemplary Construction and Correlation of the DM

Ensemble average-type power spectra can be generated. (See, e.g., References 50 and 54). The ensemble-type spectra can double frequency resolution in the physiologic range of approximately 3-12 Hz as compared to Fourier analysis. (See, e.g., Reference 55). A linear transformation derived from ensemble-type spectral analysis can be used to decompose the CFAE segments, and form data-driven orthogonal, basis vectors. (See, e.g., Reference 54). The transform equation can be, for example:

$$\begin{matrix}{\underline{a}}_{N} ( w ) = \frac{1}{n} {T}_{N \times N} ( w ) \cdot {\underline{x}}_{N} & ( 52 ) \\ n = int \frac{N}{w} & ( 53 )\end{matrix}$$

where a_{w} can be the basis vectors, N can be a dimension, ‘int’ can be the integer function, and the transformation matrix can be given by, for example:

$${T}_{N \times N} ( w ) = [ \begin{matrix}{I}_{w} & {I}_{w} & … & {I}_{w} \\ {I}_{w} & {I}_{w} & … & {I}_{w} \\ … & & & \\ {I}_{w} & {I}_{w} & … & {I}_{w}\end{matrix} ] \qquad ( 54 )$$

where T_{w} constructs repeating ensemble averages e_{w} from successive segments of signal x_{N}, with each ensemble average having a length w. The basis can be orthogonal for values of segment length w lacking integer relationships. For this exemplary study, basis vectors can be constructed for w=w*, where, for example:

$${w}^{*} = \frac{sample rate}{DF} \qquad ( 55 )$$

with the sample rate of the acquisition system being approximately 977 Hz. The DM can be defined as the ensemble average e_{w*} at the DF, used to construct basis vector a_{N} (w*).

The DM of the two 8 s pairs for each 16 s CFAE recording can be correlated. The 1^{st} and 2^{nd} segment DMs can differ when the DFs of each segment can differ, and even when the DF values can be the same, because different signal segments can be used to form each ensemble average. The normalized correlation coefficient can be given by, for example:

$$cc = \frac{{\underline{e}}_{1} ( 1 \text{:} {w}^{*} ) \cdot {\underline{e}}_{2} ( 1 \text{:} {w}^{*} )}{\sqrt{[ ( {\underline{e}}_{1} ( 1 \text{:} {w}^{*} ) \cdot {\underline{e}}_{1} ( 1 \text{:} {w}^{*} ) ) ( {\underline{e}}_{2} ( 1 \text{:} {w}^{*} ) \cdot {\underline{e}}_{2} ( 1 \text{:} {w}^{*} ) ]}} \qquad ( 56 )$$

where e₁ can be the ensemble average of the 1^{st} 8 s segment at its DF, and e₂ can be the ensemble average of the 2^{nd} 8 s segment at its DF, w* can be the ensemble average length for the 1^{st} 8 s segment, and ‘•’ can denote the inner product. Thus to compute cc, e₂ can be concatenated when it can be longer than e₁, and it can be padded with zeros when shorter than e₁.

When the DFs can be the same, the lengths of e₁ and e₂ can be the same. Normalized correlation coefficients can range from approximately 0 (e.g., no correlation) to approximately 1 (e.g., perfect correlation) and to-approximately 1 (e.g., perfect inverse correlation). Because e₁ and e₂ may not be phase aligned, the phase-optimal normalized cc can be used for comparison, for example:

$$cc = \frac{{\underline{e}}_{1} ( 1 \text{:} {w}^{*} ) \cdot {\underline{e}}_{2 φ} ( 1 \text{:} {w}^{*} )}{\sqrt{[ ( {\underline{e}}_{1} ( 1 \text{:} {w}^{*} ) \cdot {\underline{e}}_{1} ( 1 \text{:} {w}^{*} ) ) ( {\underline{e}}_{2 φ} ( 1 \text{:} {w}^{*} ) \cdot {\underline{e}}_{2 φ} ( 1 \text{:} {w}^{*} ) ]}} \qquad ( 57 )$$

where Φ can denote the phase of a₂ that maximizes cc. To construct e_{2Φ}, the ensemble average e₂ can be adjusted by wrapping around the origin as needed to align it with e₁ for maximum correlation.

This can be performed, for example, automatically via an exemplary software procedure that programs a computer processor arrangement according to an exemplary embodiment of the present disclosure. Since the ensemble averages can be used as periodic component to construct the basis vectors, their start and endpoints can be arbitrary. Normalized correlation coefficients calculated using Eq. (38) can be tabulated for all data, and separately for PV and a trial free wall (“FW”). As a check on the sequence length for analysis, the measurements can be repeated using the 1st and 2nd 4 s segment from the same data. All results can be presented as mean±standard deviation, and as coefficients of variation (e.g., standard deviation divided by mean).

For comparison, the CFE-mean and the Interval confidence level (“ICL”) parameters can be calculated. The CFE-mean can be defined as the average time duration between consecutive electrogram deflections during a specified time period. (See, e.g., Reference 56). Exemplary parameters can include: (1) a refractory period (e.g., minimum) of approximately 30 ms between counted deflections, (2) absolute peak values within the range of approximately 0.015 and approximately 0.5 mV, (3) a maximum deflection duration of approximately 10 ms, and (4) a time period of approximately 8 s. The ICL can be defined as the number of intervals between approximately 50 ms and approximately 120 ms for counted electrogram deflections. (See, e.g., Reference 57). CFAE deflections can be counted if their absolute peak values can be within the range of approximately 0.015 and approximately 0.5 mV, and the count can be done over approximately 8 s intervals.

### Exemplary Statistical Calculations

Exemplary means of all data for paroxysmal versus persistent CFAE, and separately for PV and FW sites, can be compared using the Mann-Whitney rank sum test (e.g., SigmaPlot 2004 for Windows Ver. 9.01, Systat Software, Chicago, and MedCalc ver. 9.5, 2008, MedCalc Software bvba, Mariakerke, Belgium). A value of approximately p<0.05 can be considered significant.

### Further Exemplary Results

Examples of CFAE in persistent AF are shown in the exemplary graphs of FIGS. 32A-32D. The left superior, left inferior, right superior and right inferior pulmonary vein recordings are shown. In each trace, there can be little or no isoelectric interval and the large deflections can be time-varying. There can be some periodicity evident in each of the signals, particularly as shown in the exemplary graphs of FIGS. 34A, 34C and 34D. For example, only the first 1000 sample points, approximately 1 s, can be provided so that the electrogram detail can be observed. The 1st (e.g., line 3300 of the exemplary graphs of FIGS. 33A-33D) and 2nd (e.g., line 3305 of the exemplary graphs of FIGS. 33A-33D) 8 s DMs for these CFAE are shown in FIGS. 33A-33D, and have been adjusted for optimal phase alignment based on the cross-correlation. Since the DFs of the 1st versus 2nd 8 s segments may not be the same, the lengths w* of the pair of vectors, given by Eq. (36), may not be the same (e.g., FIGS. 33B and 33D). There can be similarity of the pairs, particularly for the graphs of FIGS. 33A, 33C and 33D, which can have more periodic CFAE that can be observed in the exemplary graphs of corresponding FIGS. 32A, 32C and 32D. The correlation of the DM pairs can be given by the value of cc_{φ} at the lower right in each of FIGS. 33A-33D, as calculated using Eq. (38). Although there may not a great deal of overlap for the DM of FIG. 33C, there can be a high degree of correlation. This can be because the amplitudes and baseline levels of the traces can be normalized by Eq. (38). Since the shapes of the traces in the graph of FIG. 33C can be otherwise quite similar, there can be a large correlation. In contrast, normalization of the y-axis shift or scale can still not provide good overlap for the traces the graph of FIG. 33B, and the ccΦ can be substantially lower, (e.g., about 0.378).

Examples of CFAE in paroxysmal AF are provided in the graphs of FIGS. 34A-34D. As in the exemplary graphs of FIGS. 32A-32D, the left superior, left inferior, right superior and right inferior pulmonary vein recordings are shown. In each tracing, there can be no isoelectric segment and the large deflections change even more drastically over time as compared to the tracings for persistent AF in the graphs of FIGS. 32A-32D. There can be some periodicity to the signals of FIGS. 34B and 34C; however the large deflections can change dramatically in shape from one cycle to the next. The corresponding 1st and 2nd 8 s ensemble averages at the DF for these CFAE are given in the exemplary graphs of FIGS. 35A-35D. There can be partial overlap in the traces of the graphs of FIGS. 35A-35D, particularly for those with greater CFAE periodicity (e.g., FIGS. 34B and 34C). There can be less correlation of the DM in the graph of FIGS. 35A-35D for the paroxysmal CFAE, as compared with those of persistent CFAE in the graphs of FIGS. 33A-33D. The periods of the DM for the paroxysmal data (e.g., FIGS. 35A-35D) tend to be longer as compared to the persistent AF ensemble averages (e.g., FIGS. 33A-33D), meaning that the DFs in paroxysmal AF tend to be lesser in frequency, as calculated using Eq. (36).

Examples of power spectra are shown in the exemplary graphs of FIGS. 36A-36D. These spectra can all be generated from CFAE acquired from the right superior pulmonary vein ostia. FIGS. 36A and 36B show the 1st and 2nd 8 s for persistent AF, can be generated from the CFAE of FIG. 32C. FIGS. 36C and 36D show graphs of the 1st and 2nd 8 s for paroxysmal AF, and can be generated from the CFAE of FIG. 34C. The persistent AF spectra can be less complex. The DF can be evident at approximately 6.7 Hz, and can be maintained in both of FIGS. 36A and 36B. There can be a prominent sub-harmonic at approximately 3.35 Hz in both FIGS. 36A and 36B. The paroxysmal AF spectra, however, tend to be more complex. There can be a higher and more complex spectral background level, as well as several prominent peaks, particularly in FIG. 36D. The DF can shift from approximately 5.6 Hz in the graph of FIG. 2762C to approximately 5.4 Hz in FIG. 36D. The prominent peak at approximately 11-11.2 Hz can be the second harmonic.

For the particular examples given in the exemplary graphs of FIGS. 32A-36D, which can be representative of all persistent and paroxysmal CFAE data, there can be more instability in paroxysmal AF frequency components. This can result in less correlation between DM pairs in paroxysmal AF, as the DM can be more changeable from the 1st to 2nd 8 s segment (e.g., more time-varying).

Classification based on the DM correlation coefficients is shown in the exemplary graphs of FIGS. 37A and 37B. In the exemplary graph of FIG. 31A, e.g., only the mean values of the correlation coefficients for all recording sites is shown for each patient. There can be 10 persistent and 9 paroxysmal AF patients. These values can be plotted versus patient number for clarity, and the best linear discriminant function can be shown to separate the persistent versus paroxysmal data points. Using this function, only one paroxysmal patient can be classified incorrectly. For means versus coefficients of variation (e.g., see FIG. 37B), 1 paroxysmal and 1 persistent patient can be classified incorrectly. Thus using the means alone can provide for better classification.

### Exemplary Summary Statistics

The summary data for all CFAE is shown in Table 9 below, and follows the results shown in the exemplary graphs of FIGS. 32A-36D. The mean value of the normalized correlation coefficient in both paroxysmal and persistent AF, as calculated using Eq. (38), is shown for all data, and separately for pulmonary vein and free wall data. In each case, there can be greater correlation between DM in persistent CFAE as compared with paroxysmal CFAE. Highly significant differences in the correlation coefficients of DMs for persistent AF, versus the correlation coefficients for paroxysmal AF, can be present for all data combined and for pulmonary vein data.

TABLE 9
Normalized Correlation Coefficients
for Dominant Morphology - Pooled Data

| Data | Persistent AF | Paroxysmal AF | Significance |
|---|---|---|---|
| All | 0.619 ± 0.219 | 0.502 ± 0.190 | P < 0.001 |
| PV | 0.616 ± 0.211 | 0.461 ± 0.181 | P < 0.001 |
| FW | 0.625 ± 0.238 | 0.582 ± 0.186 | P = 0.345 |

All—pooled data from all locations
PV—data from the ostia of the pulmonary veins only
FW—data from the left atrial free wall only

The normalized correlation coefficients at individual anatomic locations are indicated in Table 10 below. At each location, the DM from 1^{st} and 2^{nd} 8 s segments of each CFAE can be more correlated for persistent AF data. The significance of the difference in mean correlation values can be given by the P value. There can be a significant difference at the left superior pulmonary vein (e.g., approximately p<0.001) and at the right superior pulmonary vein (e.g., approximately p<0.05).

TABLE 10
Normalized Correlation Coefficients
for Dominant Morphology by Location

| Location | Persistent | Paroxysmal | Significance |
|---|---|---|---|
| LSPV | 0.663 ± 0.202 | 0.414 ± 0.153 | P < 0.001 |
| LIPV | 0.557 ± 0.187 | 0.454 ± 0.179 | P = 0.155 |
| RSPV | 0.654 ± 0.201 | 0.500 ± 0.185 | P = 0.048 |
| RIPV | 0.591 ± 0.247 | 0.478 ± 0.209 | P = 0.127 |
| ANT | 0.674 ± 0.220 | 0.647 ± 0.163 | P = 0.579 |
| POS | 0.575 ± 0.250 | 0.518 ± 0.191 | P = 0.425 |

LSPV—left superior pulmonary vein,
LIPV—left inferior pulmonary vein,
RSPV—right superior pulmonary vein,
RIPV—right inferior pulmonary vein,
ANT—anterior left atrial free wall,
POS—posterior left atrial free wall.

The exemplary values of normalized correlation coefficients by patient are shown in Table 11 below. The mean values averaged for persistent and paroxysmal AF can correspond to the mean values in Table 9, all data, except for rounding. The coefficients of variation are also provided. There can be more spatial variability in the correlation between DM for paroxysmal AF patients (e.g., average coefficient of variation of approximately 0.379) as compared to persistent AF patients (e.g., average coefficient of variation of approximately 0.345) but this difference may not rise to the level of significance.

TABLE 11
Normalized Correlation Coefficients
for Individual Patients - All Data

| Type | |
|---|---|
| Persistent AF | Paroxysmal AF |

| Patient | Mean | COV | Mean | COV |
|---|---|---|---|---|
| 1 | 0.707 | 0.298 | 0.495 | 0.432 |
| 2 | 0.577 | 0.180 | 0.475 | 0.393 |
| 3 | 0.564 | 0.311 | 0.537 | 0.308 |
| 4 | 0.705 | 0.298 | 0.454 | 0.387 |
| 5 | 0.587 | 0.574 | 0.529 | 0.325 |
| 6 | 0.699 | 0.256 | 0.471 | 0.378 |
| 7 | 0.561 | 0.290 | 0.672 | 0.454 |
| 8 | 0.606 | 0.434 | 0.451 | 0.338 |
| 9 | 0.510 | 0.402 | 0.483 | 0.394 |
| 10 | 0.618 | 0.405 | — | — |
| MN | 0.613** | 0.345† | 0.507** | 0.379† |
| SD | 0.068 | 0.111 | 0.069 | 0.048 |

COV—coefficient of variation
**P = 0.004,
†P = not significant

In Table 12 below, the results for 4 s data are indicated. As for the 8 s comparisons of Table 11, mean DM correlation coefficients can be greater for persistent AF as compared with paroxysmal AF data. However there can be no significant differences, suggesting that 8 s of data can provide better results. In Tables 13A and 13B below, the results using CFE-Mean and ICL are shown. For the 1^{st} versus the 2^{nd} 8 s, there can be moderately significant differences in persistents versus paroxysmals for the 2^{nd} 8 s segment, ICL and CFE-Mean parameters (e.g., approximately p<0.05), as shown in Table 13A below. When the data from both 8 s segments can be combined, there can again be moderate significance differences between persistents and paroxysmals, ICL and CFE-Mean parameters (e.g., approximately p<0.05), shown in Table 13B below.

TABLE 12
Normalized Correlation Coefficients for Individual
Patients - All Data 4096 points (4 s)

| Type | |
|---|---|
| PersistentAF | ParoxysmalAF |

| Patient | Mean | COV | Mean | COV |
|---|---|---|---|---|
| 1 | 0.622 | 0.371 | 0.511 | 0.540 |
| 2 | 0.498 | 0.283 | 0.513 | 0.355 |
| 3 | 0.581 | 0.303 | 0.482 | 0.405 |
| 4 | 0.676 | 0.374 | 0.537 | 0.322 |
| 5 | 0.430 | 0.751 | 0.581 | 0.164 |
| 6 | 0.648 | 0.313 | 0.540 | 0.462 |
| 7 | 0.601 | 0.214 | 0.508 | 0.247 |
| 8 | 0.671 | 0.339 | 0.473 | 0.154 |
| 9 | 0.553 | 0.148 | 0.724 | 0.103 |
| 10 | 0.586 | 0.327 | — | — |
| MN | 0.587 | 0.343 | 0.541 | 0.306 |
| SD | 0.077 | 0.160 | 0.076 | 0.150 |

COV—coefficient of variation
P = not significant

TABLE 13A
CFE-mean and ICL (8 s intervals)

| Type | ICL - 1st 8 s | ICL - 2nd 8 s | CFE-M 1st 8 s | CFE-M 2nd 8 s |
|---|---|---|---|---|
| Persistent AF | 94.24 ± 18.48 | 94.85 ± 18.65 | 89.14 ± 20.39 | 88.89 ± 21.66 |
| Paroxysmal AF | 91.23 ± 19.65 | 90.17 ± 19.00 | 95.62 ± 42.30 | 92.30 ± 22.44 |
| Significance | P = 0.255 | P = 0.027 | P = 0.237 | P = 0.043 |

TABLE 13B
CFE-mean and ICL (two 8 s intervals)

| Type | ICL (# of deflections per 8 s) | CFE-M (ms) |
|---|---|---|
| Persistent AF | 94.54 ± 18.52 | 89.01 ± 20.99 |
| Paroxysmal AF | 90.70 ± 19.28 | 93.96 ± 33.81 |
| Significance | P = 0.020 | P = 0.025 |

In this exemplary study, the concept of a dominant morphology can be introduced for CFAE signals. The DM can be defined as the ensemble average of signal segments at the dominant frequency. The DM can be representative of the basic shape of the CFAE from one electrical activation interval to the next. The DM can appear more or less like the original CFAE, depending on the periodicity and regularity of the CFAE deflections, and the degree to which the DF dominants the power spectrum. DM can be compared from the 1^{st} to the 2^{nd} 8 s segments of CFAE recordings as an estimate of the temporal stability of the time and frequency components. For example, at all individual anatomic locations, and for the left atrium as a whole, there can be a greater correlation between the two segments in persistent AF. The greater correlation can mean that the DM can be more temporally stable in persistent CFE as compared to paroxysmal CFAE. The difference in exemplary correlation can be highly significant for left superior pulmonary vein recordings and moderately significant for the right superior pulmonary vein recordings. It can also be found that the spatial variation in correlation of DM pairs, in terms of the COV, can be greater in paroxysmal CFE than in persistent CFAE data, although these values may not rise to the level of significance (e.g., Tables 11 and 12). The greater spatial variation in paroxysmal AF can suggest that there can be a less centralized and consistent source driving AF in these patients.

### Exemplary Clinical Correlates

In this exemplary study, the exemplary concept of the DM can be introduced and tested. The DM can add to the arsenal of descriptors that have been developed to characterize CFAE, which can now include the dominant amplitude, and the mean and standard deviation in spectral profile. The DM can be a combined morphologic and frequency descriptor. The use of DM and DA can provide information about the morphologic detail at the DF. These parameters can go beyond the DF measurement, by characterizing CFAE shape according to the morphology of the main frequency component, rather than measuring frequency itself. The DA, but not the DM, can be extracted using the Fourier transform. The Fourier basis can be a general basis consisting of sinusoids. Each Fourier frequency component, a sinusoid, can have an amplitude and frequency, but it may not have an associated morphology. The DM extracted using ensemble averaging can provide the morphology of the CFAE at the DF. It can represent the main periodic shape of the signal, which can sometimes, but not always, be identifiable in the original CFAE data (e.g., FIGS. 32A-35D).

By knowing the spatiotemporal variability in the DM, it can be possible to infer the stability of drivers of electrical activation in the vicinity of the recording electrode. It can be expected that stable drivers can have spatiotemporal stability in the DM, that can be a high degree of correlation from the 1^{st} to the 2^{nd} 8 s recordings, and a similar high degree of correlation at spatially distinct recording locations. A site with the highest degree of temporal correlation can be proposed to be a candidate catheter ablation site, since the stability of such a site can likely be indicative of a stable driver in the vicinity of the recording electrode. Since such sites can appear to exist more commonly in persistent AF, it can be possible that ablation at a subset of CFAE recording sites in these patients can do as well to eliminate AF as compared with ablation at all CFAE recording sites. Such a constraint can be helpful to reduce morbidity. Areas where DM can be stable that also have high DF (see, e.g., Reference 59) can be of particular interest for catheter ablation.

Other ensemble average vectors besides the DM could be compared to provide additional information. For example it might be useful to compare the morphology of ensemble averages at other tall peaks in the frequency spectrum, which can be indicative of secondary, independent drivers of electrical activity. Although comparisons can be made from the 1st 8 s to the 2nd 8 s and from the 1st 4 s to the 2nd 4 s of a 16 s time series, comparisons could also be made over longer or shorter time intervals, as well as between segments disparate in time. For example the long-term stability of the DM could be estimated by extracting 8 s segments from CFAE that can be separated by 1 minute or more in time.

The exemplary study can be done using CFAE recordings from a limited number of sites and a limited number of patients. DM can be defined as the ensemble average at the DF. The DF can change from the 1st to the 2nd 8 s segments. Thus for simplicity, DF ensemble averages which could have differing vector lengths can be compared. Although comparisons of ensemble averages at the same frequency can have some relevance (e.g., use of the DF of the 1st 8 s segment to also extract the ensemble average of the 2nd 8 s segment for comparison), slight temporal shifts in DF over 16 s can be common. Therefore, the power at the frequency of the DF in one 8 s segment can shift to low levels in the other 8 s segment, as for example in FIG. 12C versus 12D, which can likely render such a comparison less relevant.

### Further Exemplary Conclusions

The DM can be extracted and compared in paroxysmal and persistent CFAE. There can be higher temporal variability in the DM of paroxysmal CFAE. There can also be higher spatial variability in the DM of paroxysmal CFAE, although this may not rise to the level of significance (e.g., Tables 11 and 12). Extraction and analysis of the DM can show that it can be useful to compare and contrast electrogram morphology at the DF so that CFAE in paroxysmal versus persistent AF patients can be compared. The greater spatiotemporal variability in paroxysmal AF can be suggestive of more instability in the electrical activity of these patients. The greater spatiotemporal correlation in persistent CFAE can be suggestive of the presence of more stable, intransigent drivers of AF in these patients, perhaps due to structural remodeling. The DM, as well as ensemble averages arising from secondary peaks in the frequency spectra, can be indicative of the morphology and characteristics of AF drivers of electrical activity that can be sought for catheter ablation.

### Exemplary Spectral Estimator

An exemplary embodiment of apparatus, method, and computer-readable medium, which can be configured to provide a spectral estimating (which can be referred to herein interchangeably as the “spectral estimator” or the “estimator”) can be based upon a mathematical transform for signal averaging. This estimator can compute the power spectrum of a signal, which can display the frequency content in terms of the magnitude of the periodic components. The exemplary estimator can be computed by sampling properties of the autocorrelation function. While the exemplary estimator may share some similarities with a Fourier transform, it can model different components of the autocorrelation function as compared with the Fourier transform. A Fourier transform is not very useful for real time analysis, because a Fourier transform should be recomputed for each new time window that is to be analyzed. For example, if updates are desired using each new discretized sample point, it would be difficult to update the Fourier transform in real time, and a powerful computer would be needed for this purpose. If the frequency spectra of multiple channels are all expected to be updated in real time, it may not be possible to perform this using the Fourier transform, particularly as the number of the multichannels increases.

Multichannel biomedical data is becoming increasingly common because investigators and clinicians find it useful to have more data. The extra data usually comes from more spatial recordings, corresponding to spatial resolution increases. This can be helpful for many types of clinical analyses including analysis of the heart and gastrointestinal system. It can be easier to detect an abnormality when the spatial resolution increases. The frequency content of recorded signals can also be analyzed. The frequency content can be more robust to noise and motion artifact as compared with the temporal content of the signal.

The largest magnitude frequencies can be related to drivers of the particular biological system being analyzed (e.g., higher frequencies in the main peaks of the spectrum suggest that events are occurring more rapidly). Although the Fourier Transform parameters may not be reused during real time updates, the parameters of the exemplary spectral estimator can be reused. The exemplary spectral estimator can function by signal averaging. The power spectrum using the exemplary spectral estimator can be constructed by taking the ensemble average of signal segments at many segment lengths. To produce one power spectrum, the exemplary spectral estimator can be slower than the Fourier Transform (e.g., computation time is about twice as long). However, if the exemplary spectral estimator power spectrum is then updated on the next sample point, because it can be based on signal averaging, the new data point can be easily averaged using a few computational arithmetic operations. By comparison, a Fourier Transform should compute everything all over again. For real time update, the exemplary spectral estimator can be about 150 or more times faster than the Fourier transform. Thus, for any particular computer system, when the Fourier transform reaches its limit in terms of how many channels can be analyzed in real time, the exemplary spectral estimator can analyze 150× more channels (e.g., in terms of the software implementation of the various transforms).

Furthermore, it can be possible to implement a real-time version of the exemplary spectral estimator in hardware, for example, using integrated circuits on a circuit board. Schematically, the integrated circuits can be used, along with connections to other integrated circuits, and some of the basic timing, to enable correct handshaking between the data and addressing streams. This can be significant because real time implementation of the Fourier Transform in hardware may not be possible. However, the exemplary spectral estimator circuitry may not require computational supervision. Thus, it can be implemented as a standalone circuit board. This is also significant because most or all spectral analyzer boards utilize input from a computer, and expensive commercial proprietary software is used on the computer to control the spectral analyzer board. With the exemplary spectral estimator implementation, no computer or software can be needed.

### Exemplary Method

### Exemplary Spectral Estimator Clinical Data Acquisition

Atrial electrograms were recorded from patients referred to the Columbia University Medical Center cardiac electrophysiology laboratory for catheter ablation of the atrial fibrillation (“AF”) substrate. Ten patients had documented clinical paroxysmal AF. Ten other patients had longstanding persistent AF that did not terminate for several months to many years prior to catheter mapping and ablation. The exemplary atrial mapping procedure was done using a NaviStar ThermoCool catheter, 7.5 F, with 3.5 mm tip and an about 2 mm spacing between the bipoles of the distal ablation electrode (e.g., Biosense-Webster Inc., Diamond Bar, Calif., USA). The electrogram signals were acquired using a General Electric CardioLab system (e.g., GE Healthcare, Waukesha, Wis.), and filtered at acquisition from about 30 to about 500 Hz with a bandpass filter (e.g., single-pole) to remove baseline drift and high frequency noise. The filtered signals were then digitally sampled at 977 Hz and the digital data was stored. As in previous exemplary studies, to standardize the morphological characteristics, all CFAE's were preprocessed to mean zero and unity variance (e.g., average signal level=0 volts, standard deviation and variance=1).

### Exemplary Spectral Estimator Computer Implementation

For an exemplary computer implementation of the NSE, a software procedure and a hardware design were developed and first the ensemble means of signal segments were calculated. The segment length can be a period w. When exemplary successive signal segments of length w can be added together, the sum can reinforce the individual components if they can be correlated, for example, and if there can be a signal component with period w. For each exemplary ensemble mean vector, the square root of the sum of squares of all elements divided by the vector length can be defined as the ensemble power. The square root of ensemble power can be plotted versus frequency to form the NSE power spectrum. The frequency f can be given by the sample rate/w. The ensemble power can be computed in the standard electrophysiologic frequency range of about 3-12 Hz. (See, e.g., References 68 and 69). At the 977 Hz sampling rate, this can correspond to a range of w from about 325-81 sample points. Thus, for example, the NSE can be a 245 point spectrum.

An exemplary computer-programmed and executed procedure to implement the NSE power spectrum calculation is shown below (e.g., called spectral_estimate). This code was implemented in FORTRAN (e.g., Intel Visual FORTRAN Compiler, ver. 9, 2005), although not limited thereto. Period w ranges from n1=325 to n0=81 sample points (e.g., about 3-12 Hz). The calculation window can be n2=8192 sample points, which can be approximately 8 s when sampled at r=977 Hz. The number of signals analyzed can be n3=216. For calculation, (i) the input data array, with each patient data being received from a separate file, can be ‘inp’ (e.g., lines 4-5), (ii) the ensemble mean can be stored in array ‘en’ (e.g., lines 7-15), and (iii) the generated spectrum can be stored in array ‘s’ (e.g., lines 16-19). For faster run time, lines 8-12 can calculate the ensemble means only from w=162 to w=325. Whereas, in lines 13-15, the ensemble means can be calculated from, e.g., w=162 to w=81 by simply adding the two halves of vector 2w that can be calculated by lines 8-12, which can result in, for example:

$$[ \begin{matrix}{e}_{w , 1} \\ {e}_{w , 2} \\ … \\ {e}_{w , w}\end{matrix} ] = [ \begin{matrix}{e}_{2 w , 1} \\ {e}_{2 w , 2} \\ … \\ {e}_{2 w , w}\end{matrix} ] + [ \begin{matrix}{e}_{2 w , w + 1} \\ {e}_{2 w , w + 2} \\ … \\ {e}_{2 w , 2 w}\end{matrix} ] \qquad ( 58 )$$

which can be determined in lines 13-15, and which can reduce redundancies in the exemplary calculation.

This exemplary software program (e.g., spectral_estimate) can be executed by a computer processor, and used (when configured thereby) for non-real-time, and, for example, it can calculate a single spectrum for each patient record using sample points 1-8,192. (See, e.g., References 64-67). The offline time for computation of a single spectra for 216 patient records was compared with the radix-2 implementation of the Fast Fourier Transform (“FFT”). (See, e.g., Reference 70). The time for spectral estimation, lines 7-20 can be determined using the ‘date_and_time’ function in FORTRAN, which can be inserted between lines 6-7, and between lines 20-21 in the source code. Using the same ‘date_and_time” function, the run time for spectral estimation using offline radix-2 FFT implementation can also be determined. Since slight temporal changes in processor speed can occur, the mean and standard deviation in spectrum computation time over five trials was determined for both the NSE and FFT methods.

The exemplary spectral_estimate is shown below:

| | |
|---|---|
| | program spectral_estimate |
| 1 | parameter(n0=81,n1=325,n2=8192,n3=216,r=977.);character g*11 |
| 2 | real en(n1,n1),inp(n2,n3),s(n1); integer i,j,k |
| 3 | open(7,file=‘filename.txt’);open(9,file=‘espectrum.txt’) |
| 4 | do 6 i = 1,n3 |
| 5 | read(7,*)g;open(8,file=g);read(8,*)inp(1:n2,i);close(8) |
| 6 | continue |
| 7 | do 20 i = 1, n3 |
| 8 | do 12 j = n1/2+1, n1 |
| 9 | en(j, 1:j) = 0. |
| 10 | do 12 k = 1, n2/j |
| 11 | en(j, 1:j) = en(j, 1:j) + inp((k-1*j+1:(k-1)*j+j, i |
| 12 | continue |
| 13 | do 15 j = n1/2, n0, -1 |
| 14 | en(j, 1:j) = en(2*j, 1:j) + en(2*j, j+1:2*j) |
| 15 | continue |
| 16 | do 19 j = n0, n1 |
| 17 | s(j) = sqrt(sum(en(j, 1:j)**2)/n2) |
| 18 | if(i.eq.4) write(9,*)r/j,s(j) |
| 19 | continue |
| 20 | continue |
| | stop;end |

The exemplary NSE and FFT spectral estimators were then implemented for real-time analysis. For simplicity, an update was computed once every sample point (e.g., moving size M=1). Thus, the first snapshot for the exemplary spectral analysis can include sample points 1-8192 in each electrogram, the second snapshot consisted of points 2-8193, up to the final snapshot which consisted of points 8192-16383. Examples are shown in FIGS. 42A and 42B. FIG. 42B shows an exemplary graph providing ranges from sample point 2 to sample point 8193. FIG. 42A shows an exemplary graph providing ranges from sample point 12 to sample point 8203. The code for NSE analysis is shown below (e.g., spectral_estimate_real-time). The declaration lines can be virtually the same as for the offline program (e.g., spectral_estimate). In the real-time program, most variables can be vectors whose elements can represent the range in w from 81 to 325. Declaring length w as an integer can considerably reduce computation time as compared to a floating point declaration. Lines 5-8 herein above can compute constants c1 and c2 for a moving average (e.g., low pass filter), which can then be used on each pass of the calculation that follows in the code sequence. Lines 9-11 can input the data, the same as lines 4-6 in spectral_estimate; however, the ensemble mean calculation (e.g., lines 12-18) can be slightly different as compared with the exemplary spectral_estimate program. The actual exemplary calculations can be done in lines 15-17. The index ‘ind’ can first be calculated (e.g., line 15), which can be used to point to a particular element for each vector that can be used for calculation. The prior value of the ensemble mean at the index can first be stored in a buffer (e.g., line 15). If the index can exceed the value of w, it can wrap around to a value of 1. The prior value of the ensemble mean at index can also be stored in a buffer (e.g., ee), and can then be updated using a moving average (e.g., line 16), which can include constants c1 and c2 calculated in lines 5-8. The exemplary real-time ensemble mean calculation can be expressed as, for example:

e_{w, i}=c1·e_{w, i-1}+c2·x_{w, i} (59)

where:

$$\begin{matrix}c 1 = \frac{n - 1}{n} \\ and & ( 60 ) \\ c 2 = \frac{1}{n} & ( 61 )\end{matrix}$$

with n, c1, and c2 being dependent upon the value of w in

$$n = int ( \frac{N}{w} ) \qquad ( 62 )$$

The power of the ensemble mean vector of length w can then be updated in line 17. The ensemble power can be expressed as, for example:

P_{w}=e₁·e₁+e₂·e₂+ . . . +e_{w}·e_{w} (63)

where e can be the scalar error, index i can range from 1 to w, and the divide by w can be accounted for when the spectral point can be subsequently calculated in the exemplary code below. At any particular index value i−1 to w, the prior squared error e_{i-w}*e_{i-w} which can be calculated w sample points previously in time, can be removed, and can be replaced with the newly calculated eᵢ*eᵢ. Thus, at each index i, the power can be updated as, for example:

P_{w}=P_{w}−e_{i-w}·e_{i-w}+eᵢ·eᵢ (64)

where the index i can be incremented with each new input sample point. For each new sample point, all P_{w} can be updated using Eq. (64). All spectral points S_{w} can then be calculated as the square roots of P_{w}, scaled by

$$\frac{n ( w )}{\sqrt{N}}$$

$$with$$

$$\frac{1}{\sqrt{N}}$$

being symbolized by xn in lines 1 and 17 of the exemplary spectral_estimate real time program. Using

$$\sqrt{n} {p}_{wRMS} = \frac{n}{\sqrt{N}} \sqrt{{e}_{w}^{T} {e}_{w}}$$

rather than

$$\sqrt{n} {p}_{wRMS} = \frac{\sqrt{N}}{W} \sqrt{{e}_{w}^{T} {e}_{w}} ,$$

such that there is no actual ‘divide by’ in the computation which can reduce the runtime considerably. One complete spectrum, arbitrarily selected as that for data record 4, can be stored for subsequent analysis (e.g., in lines 19-22). The frequency variable, which can be the x-axis parameter for graphing, can also be stored (e.g., as r/w, line 21).

The exemplary spectral_estimate_real_time is shown below:

| | |
|---|---|
| | program spectral_estimate_real_time |
| 1 | parameter(n0=81,n1=325,n2=16384,n3=216,xn=0.01105,r=977.) |
| 2 | integer i,i\|1,ind(n1),n(n1),w; character g*11 |
| 3 | real c1(n1),c2(n1),e(n1,n1),ee,inp(n2,n3),p(n1),s(n1) |
| 4 | open(7,file-‘filename.txt’);open(9,file=‘espectrum.txt’) |
| 5 | do 8 w=n0,n1 |
| 6 | n(w)=int(real(n2)/(2.*w)) |
| 7 | c1(w)=real(n(w)-1)/real(n(w));c2(w)=1/real(n(w)) |
| 8 | continue |
| 9 | do 11 i1 = 1,n3 |
| 10 | read(7,*)g;open(8,file=g);read(8,*)inp(1:n2,i1);close(8) |
| 11 | continue |
| 12 | do 23 i1 = 1, n3 |
| 13 | ind=0;do 23 i=1,n2 |
| 14 | do 18 w = n0, n1 |
| 15 | ind(w)=ind(w)+1;if(ind(w).gt.w)ind(w)=1;ee=e(w,ind(w))**2. |
| 16 | e(w,ind(w))=c1(w)*e(w,ind(w))+c2(w)*inp(i,i1) |
| 17 | p(w)=p(w)+e(w,ind(w))**2-ee;s(w)=sqrt(p(w))*n(w)*xn |
| 18 | continue |
| 19 | if((i.ne.8192).or.(i1.ne.4)) goto 23 |
| 20 | do 22 w=n0,n1 |
| 21 | write(9,*) r/w, s(w) |
| 22 | continue |
| 23 | continue |
| | stop;end |

In the spectral_estimate_real-time program, for example, about 16,384 spectra can be calculated for each of 216 patient records (e.g., lines 12-13). This exemplary program can be designed as a real-time implementation, updating the exemplary spectrum at each new input sample point. However, it may likely only be confirmed as a real-time procedure if the update calculations for all channels can be done at a speed faster than the sampling interval of about 1 millisecond. As for the spectral_estimate program, the time for spectral estimation using spectral_estimate_real-time was determined by inserting the ‘date_and_time’ FORTRAN library function at the appropriate lines in the code (e.g., between lines 11 and 12, and after line 23).

The radix-2 DFT program was also implemented for real time. For ease of comparison, all DFT calculations were repeated for each sliding analysis window rather than using any shortcuts. (See, e.g., References 60 and 61). Because the repetition of DFT calculations can be time-consuming, it was run for only 512 input sample points. Again inserting the FORTRAN ‘date_and_time’ function at appropriate locations in the code, the time for DFT calculation over 512 sliding windows was determined, which was then scaled by 32× for comparison with the NSE implementation in which spectra were calculated for about 16384 sliding windows. Since slight temporal changes in processor speed can occur, the mean and standard deviation in spectral computation time over five trials was determined for both the NSE and DFT real-time methods.

Lines 13-23 of the spectral_estimate_real-time program were then implemented in schematic form in hardware. Integrated circuits were selected to do the calculations shown in these lines. The integrated circuits were selected for their utility and low cost. An exemplary goal was to implement this calculation on a small prototype electronics board.

### Exemplary Results

Exemplary graphs of spectral estimates were constructed for a patient with longstanding persistent atrial fibrillation (“AF”). The spectral estimates were generated from CFAE's recorded using a bipolar contact electrode. In FIGS. 38A-38C, instances are shown of the spectral estimate using the original NSE procedure called spectral_estimate (see e.g., FIGS. 38A and 38C) and the real-time NSE procedure called spectral_estimate_real-time (see e.g., FIGS. 38B and 38D). FIGS. 38A and 38B show a recording from the left superior pulmonary vein antrum in a patient with persistent AF, and FIGS. 38C and 38D were recorded from the left inferior pulmonary vein antrum in the same patient. For spectral_estimate, the result of analysis of the window from data points 1-8192 is shown. For spectral_estimate_real-time, the result of analysis at sample point 8192 is shown (e.g., since the analysis windows can be moving averages). There can be virtually no difference in the result, with the spectra at left versus right appearing very similar. Therefore, the real-time implementation can provide a similar spectral estimate to the non-real-time implementation at the same data point. To illustrate how the time-varying spectral content can be detected using the real-time procedure, FIGS. 39A-40D show spectra from subsequent points in time for data acquired at the posterior left atrial free wall in a different persistent AF patient. The split peaks at the dominant frequency can change slightly over short time intervals (e.g., approximately 50-100 milliseconds, for example, FIG. 39A-39D). Over longer intervals, major changes in the spectra can occur (e.g., approximately 1000 milliseconds, for example, FIG. 40A-40D). The split peaks at the dominant frequency (e.g., k=12,000 and 13,000 sample points) can become single peaks (e.g., k=14,000 and 15,000 sample points). The background level can become somewhat less particularly at lower frequencies for the spectra acquired at k=15,000 sample points. Thus temporal changes in spectra details can be evident over both short and longer intervals using the real-time implementation of the exemplary NSE.

### Exemplary Summary

Software Statistics Computation of single 8192-point spectra for 216 patient records was completed in about 0.38±0.01 s using the exemplary NSE and in about 0.12±0.01 s using the radix-2 DFT implementation. Thus the single DFT calculation can be 3-4 times faster than the exemplary NSE. This can be explained by the increased number of calculations needed for a single spectral estimate by the exemplary NSE as compared to DFT. For real-time analysis, about 16,384 spectra from 216 patient records (e.g., 3,538,944 spectra total) were calculated in about 11.63±0.14 s for NSE, while 512 spectra for 216 patient records (e.g., 110,592 spectra in total) were calculated in about 55.79±0.26 s for the radix-2 DFT implementation. Scaling by 32×, 3,538,944 spectra can be calculated in about 1785.28±8.32 s for DFT. This can represent a speed advantage about 153:1 of the exemplary NSE over DFT when the real-time analysis procedures can be implemented. Dividing each time by 3,538,944, the average time for a single real-time spectral calculation was about 3.29 μs for the exemplary NSE versus about 504.5 μs for DFT. Over a 1 millisecond sampling period, the exemplary NSE procedure had the capability to spectrally analyze a maximum of about 303.95 data channels, although only 216 patient data sequences were actually analyzed. By comparison, the DFT procedure could only analyze a maximum of about 1.98 channels, which for purposes of calculation means that the DFT can only analyze a single recording sequence in real time within a 1 millisecond interval.

### Exemplary Hardware Implementation

An exemplary schematic diagram for a hardware implementation for an apparatus and/or a system according to an exemplary embodiment of the present disclosure based upon the NSE real-time software procedure spectral_estimate_real-time is shown in FIG. 41. The exemplary hardware can include a mix of analog and digital components, and for simplicity, it can sample the data at exactly 1.0 kHz (e.g., 1 millisecond intervals). The exemplary system/apparatus can utilize a 1 kHz clock 4105 for sampling the data stream. To update parameters from, e.g., w=81-325 corresponding to a 12-3 Hz frequency range, which can include 245 variables, a 245 kHz clock 4110 was used. An off-the-shelf 250 kHz oscillator can also be used. The data stream (e.g., signal) can be tracked and then held using an edge of, for example, the 1 kHz clock 4105. The input can then be valid until the next 1 kilohertz pulse. The edge of the 1 kHz clock waveform can also be used to reset a counter (e.g., wcounter) 4115. This counter can provide the current value of segment length w being analyzed, and it can count in increments of 1 from 81 to 325 on each pulse from the 245 kHz clock. The wcounter 4115 output can be used as addressing information for the memory chips that can be present on the electronics board.

In the exemplary schematic diagram of FIG. 41, addressing information is indicated by dotted lines, while data is noted by solid lines. In Table 13 below, the characteristics of each memory chip on the board are shown. The ensemble means can be stored in EMEM 4120 for each segment length w from 81-325. Each ensemble mean can be a vector of length w. Therefore the addressing information sent to the EMEM chip can specify not only the segment length w, but also the index number for that w, the latter of which can be stored in the index memory chip (“IMEM”) 4125. Each time a new input sample point can be received, the index at each w can be incremented by 1 with wraparound. For example, for w=100, the index can begin at 1, and can be increased to 2, 3, 4, . . . , 100 for each new input sample point (e.g., once every millisecond). For w=100, when the index reaches 100, it can then be reset to 1. This can be done by use of the counter 4130 and comparator 4135 that can be associated with IMEM 4125. The index value contained in IMEM 4125 for segment length w can be presented to the counter (e.g., icounter) 4130. Using the 245 kHz clock 4110 with a delay, the icounter 4130 can then be incremented by 1. The icounter 4130 output after incrementation can be compared to the current value of w (e.g., received from wcounter) 4115. If the icounter 4130 output can be greater (e.g., its value can be w+1), it can be reset to 1.

The digital ensemble mean value contained within EMEM 4120 for a particular w and index can be output to a digital-to-analog converter (“DAC”) 4140. The DAC 4140 output can be multiplied by the constant c1. The new input sample point (e.g., input) can be multiplied by the constant c2. The constants c1 and c2 can be used to construct the moving average of the ensemble mean (e.g., of spectral_estimate_real-time), according to the following exemplary equation:

E(k)=c1×ensemble mean+c2×input (65)

where, as computed in the spectral_estimate_real-time:

$$n = int ( \frac{N}{w} )$$

where N can be the window length, 8192 sample points, ‘int’ can be the integer function, and can be represented as, for example:

$$c 1 = \frac{n - 1}{n}$$

$$and$$

$$c 2 = \frac{1}{n}$$

The values of c1 and c2 can be constant for this exemplary implementation, and they can be stored in CMEM before real-time spectral analysis can be performed using a separate device to write to the chips, and to maintain the standalone quality of the electronic board design. The two products on the right-hand-side in Eq. (58) can then be summed. The result E(k) can be input to an analog-to-digital converter (“ADC” 4145), and the resulting digital value of E(k) can replace E(k−1) in EMEM 4120 for the w and index being currently pointed to by the addressing information. The moving average filter output can also be squared, which can then be used as one of the elements inputted to a summing circuit to form the ensemble power P. The update of the ensemble power P can be given by the following exemplary equation:

P(k)=P(k−1)+e(k)²−E(k−1)² (66)

The quantity P(k−1) can be accessed from the ensemble power memory chip (“PMEM” 4150). The quantity E(k−1) can be obtained by squaring the output of the EMEM DAC. The output of this exemplary summing circuit can be the analog power P(k). This value can be digitized using an ADC, and stored as the new value of P at w. The square root of P(k) can also be obtained, which can then be digitized and stored in the spectral memory (“SMEM” 4155). SMEM 4155 can include 245 digital values representing the spectral content from w=81 to 325 (e.g., 12-3 Hz). The SMEM 4155 digital output can then be sent to a display 4160. For simplicity, the spectral display device is not shown. To prevent the need to tie the spectral analyzer board to a computer, the output of SMEM 4155 can be sent, for example, to a dot matrix display with appropriate circuitry.

In total, for example, about 27 integrated circuit chips, plus delay, display circuitry, and power supply can be used to implement the real-time exemplary NSE procedure on an electronics board. The circuitry can run at, for example, nearly 250 kHz; thus, the settling time for the integrated circuits can be less than 4 microseconds, which may need high quality components. The DAC and ADC integrated circuits can be parallel input and output, respectfully, for faster throughput. Thus, single rather than dual or quad packages can be used. For accuracy, they can be at least 8 bit devices. For memory access, latching, and counting, sufficient delay can be needed to facilitate inputs to each integrated circuit chip in FIG. 41 to settle. This can be done, for example, using timer integrated circuits, and the associated resistors and capacitors, which for simplicity is not shown in the schematic.

### Exemplary Discussion

According to one exemplary variant of the present disclosure, the exemplary NSE procedure can be implemented for a real-time analysis as a software procedure, and as a block diagram for a prototypical hardware electronics board. To be implemented in real time, the spectral estimate can be updated within the time it takes for the data stream to shift by the analysis window moving size of 1 sample point (e.g., M=1), which can be 1 millisecond. The data stream consisted of sequences of retrospectively analyzed fractionated atrial electrograms from 216 patients. The exemplary NSE algorithm/procedure was found to be implementable in real time using a few lines of software code. The mean time for calculation of one power spectrum was about 3.29 μs for the exemplary NSE versus about 504.5 μs for DFT. Thus, for real-time spectral analysis, the exemplary NSE procedure was found to be approximately 185 times faster than the DFT radix-2 implementation. Based on these values, over a 1 millisecond sampling period, the NSE procedure can spectrally analyze about 303.95 data channels while the DFT procedure can only analyze a maximum of 1.98 channels. Thus, for NSE, although 216 sequences were actually analyzed, the number of sequences could be increased to about 303, while maintaining real-time calculation within the 1 millisecond window moving size. Whereas for the radix-2 DFT implementation, only a single channel can be analyzed when the moving size M=1. The rapid speed of the exemplary NSE for real-time analysis can be due to the low computational overhead in calculating the update as compared with the DFT recalculation. The NSE can be implementable in hardware using approximately 28 integrated circuits. No computer controller or digital signal processor can be needed to run the hardware implementation.

The exemplary spectral estimator can also be used for offline analysis of “big data,” which can be large volumes of data that cannot ordinarily be processed in a timely manner by conventional software programs and hardware configurations. For example, the frequency spectra of 1000 data channels can be generated, each having about a 5 minute long sequence, with about a 1 millisecond discrete time update. The runtime using the exemplary spectral estimator on a fast PC-type computer can be approximately 1 minute, whereas it could take approximately 1 hour using a standard DFT/FFT procedure. Thus there is a considerable reduction in time and computing power necessary for off-line processing utilizing the exemplary spectral estimator.

### Exemplary Spectral Estimator Clinical Correlation

Analysis of multichannel electrogram data via spectral estimation has been shown to distinguish patients with paroxysmal versus persistent atrial fibrillation using frequency gradients. (See, e.g., Reference 71). Thus, acquisition and real-time analysis of multichannel data can be potentially important to understand the localization of substrate changes that can occur during atrial fibrillation. It can also be helpful to detect optimal ablation sites to prevent recurrence of arrhythmia. (See, e.g., References 72 and 73). In a prospective setting, rather than 216 different patient sequences as was done in this exemplary study, the data can be obtained from a multichannel electrode, for example, using a noncontact (see, e.g., Reference 74) or a basket (see, e.g., Reference 75) electrode. As the number of multichannel electrode recordings can increase, the possibility of analysis of all of the channels in real time can become more remote, yet can be necessary for optimally targeting arrhythmogenic regions for ablation. Thus, the implementation of an exemplary real-time spectral estimator for multichannel data can be potentially important to improve clinical outcome.

The exemplary NSE and DFT real-time procedures were tested on retrospective data. Implementation on prospective multichannel data can be desirable to determine the speed of the spectral estimators in this exemplary setting. Still, in principle there should be no difference in speed by using 216 retrospective patient data sequences versus 216 prospective multichannel data sequences that can be acquired simultaneously. The procedures were tested with one compiler and one computer and operating system. Use of a different compiler and computer can result in somewhat different computation times. For the exemplary hardware implementation, the design was illustrated only as a schematic block diagram.

### Exemplary Conclusions

The exemplary NSE procedure according to an exemplary embodiment of the present disclosure, can be implementable with low computational cost and complexity in both hardware and software. Real-time 1 millisecond updates of the spectral estimate can be done even when many tens or hundreds of data sequences are being acquired and analyzed during the same time interval, as can be the case when using a multichannel basket or noncontact electrode. The procedure can be implemented as a standalone spectral analyzer board at a price of approximately $500 plus the cost of the display unit, without the need to interface with a computer. Although it can be possible to improve the efficiency of the DFT real-time update by several times (see, e.g., References 60 and 61), such exemplary implementations does not match the improvement gained by using the exemplary NSE real time procedure over the DFT, which was found by comparison of software procedures to be approximately 185× faster. The sampling rate using the hardware implementation can be further increased, being limited only by the settling times of the on-board components as compared with the fast clock speed. The realization of a fast spectral analysis procedure can be potentially helpful to characterize spectral transients as they occur, as well as to update, in real time, the detailed trends and gradients in the frequency content of biomedical signals that can be present over longer sequences. This can be particularly helpful when probing the tissue substrate for anomalous regions, as is the case during electrophysiologic study of AF patients.

TABLE 13
Exemplary Characteristics of the Memory Integrated Circuits

| | | | What is stored |
|---|---|---|---|
| Name | Storage Variable | Addressing | EMEM |
| EMEM | ensemble mean | 245 x w values | e.m. for each |
| | | | index at each w |
| C1MEM | constant 245 values | constant for MA | c1 |
| | | equation | |
| C2MEM | constant 245 values | constant for MA | C2 |
| | | equation | |
| PMEM | ensemble mean | 245 values | e.p. of each e.m. |
| IMEM | Index | 245 values | points to an |
| | | | element of e.m. |
| SMEM | spectrum | 245 values | spectral points |

w = segment length to compute the ensemble mean,
245 = the number of segment lengths for which the ensemble mean can be calculated (e.g., from 81 to 325),
e.m. = ensemble mean,
e.p. = ensemble power,
MA = moving average.

TABLE 14
Exemplary Parts List

| | | Function | | |
|---|---|---|---|---|
| Part | Company | Time | Settling | Quant |
| AD532 | Analog Devices | analog mult, divide, | 1 s | 7 |
| | | square, sr | | |
| SN74LS682 | Texas Instruments | CMOS comparator | 30 ns | 1 |
| AS6C1008 | Alliance Memory | CMOS SRAM | 55 ns | 6 |
| SI510 | Silicon Labs | CMOS output | 1 ns | 2 |
| | | oscillator | | |
| HA5351 | intersil | CMOS sample/hold | 64 ns | 1 |
| MAX5595 | maxim integrated | CMOS DAC 8 bit | 1 s | 5 |
| | | parallel | | |
| SN74HC393 | Texas Instruments | CMOS Counter | 100 ns | 2 |
| ADC08D1520 | Texas Instruments | CMOS ADC 8 bit | 1 s | 3 |
| | | parallel | | |

Mult = multiplication,
s r = square root,
ns = nanoseconds,
s = microseconds.
Quantity of AD532 includes 3 multiplies, 2 squares, 1 divide by, 1 square root.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### Exemplary Appendix

The following tested exemplary Fortran code can be useful to compute ensemble average power spectra from multiple CFAEs. The exemplary code can be executed in ˜1 second on a PC-type laptop computer and can be implemented in real time.

parameter (n0=50, n1=500, n2=8192, n3=216,rate=.977)

real en(n1, n1), f(n1), inp(n2, n3) , s(n1, n3)

| | |
|---|---|
| | do 1 i = 1, n3 |
| | do 2 j = n1/2+1, n1 |
| | en(j, 1:j) = 0. |
| | do 2 k = 1, n2/j |
| | en(j, 1:j) = en(j, 1:j) +inp((k-1)*j+1:(k-1)*j+j, i) |
| 2 | continue |
| | do 3 j = n1/2, n0, -1 |
| | en(j, 1:j) = en(2*j, 1:j) + en(2*j, j+1:2*j) |
| 3 | continue |
| | do 1 j = n0, n1 |
| | s(j, i) = sqrt(sum(en(j, 1:j)**2)/n2); if(i.eq.1) f(j) = rate/j |
| 1 | continue |

en = ensemble vector, rate = digital sampling rate, s = spectral magnitude, f = frequency
inp = input matrix consisting of CFAEs normalized to zero mean and standard deviation = 1.
n0, n1 = range of segment widths (n0, n1) = (50, 500). Frequency f = 977/50 - 977/500 =
19.54Hz - 1.95Hz.
n2 = number of sample points in each CFAE = 8192.
n3 = number of CFAE from which to calculate spectra = 216.
Loop 1 (inner) computes the spectrum s(j, i) based upon Eq. (11), with frequencies given by
f.
Loop 2 zeros the ensemble matrix and computes ensemble averages from (n1)/2 + 1 to n1.
Loop 3 computes ensemble averages from w = n0 to (n1)/2 by averaging the two half segments
from Loop 2 (see exemplary Eq. (21)).

### EXEMPLARY REFERENCES

The following references are hereby incorporated by reference in their entirety.
1. Ciaccio E J, Dunn S M, Akay M, Wit A L, Coromilas J, Costeas C A. Localized spatial discrimination of epicardial conduction paths after linear transformation of variant information. Ann Biomed Eng. 1994, 22:480-492.
2. Mandapati R, Skanes A, Chen J, Berenfeld O, Jalife J. Stable microreentrant sources as a mechanism of atrial fibrillation in the isolated sheep heart. Circulation. 2000, 101:194-199.
3. Nademanee K, McKenzie J, Kosar E, Schwab M, Sunsaneewitayakul B, Vasavakul T, Khunnawat C, Ngarmukos T: A new approach for catheter ablation of atrial fibrillation: Mapping of the electrophysiologic substrate. J Am Coll Cardiol 2004, 43:2044-2053.
4. Oral H, Chugh A, Good E, Sankaran S, Reich S S, Igic P, Elmouchi D, Tschopp D, Crawford T, Dey S, Wimmer A, Lemola K, Jongnarangsin K, Bogun F, Pelosi F Jr, Morady F. A tailored approach to catheter ablation of paroxysmal atrial fibrillation. Circulation. 2006 18, 113:1824-1831.
5. Niu G, Scherlag B, Lu Z, Ghias M, Zhang Y, Patterson E, Dasari T, Zacharias S, Lazzara R, Jackman W M, Po S S. An acute experimental model demonstrating two different forms of sustained atrial tachyarrhythmias. Circ Arrhythmia Electrophysiol. 2009, 2:384-392.
6. Kapa S, Asirvatham S J. Atrial Fibrillation: Focal or Reentrant or Both? Circulation: Arrhythmia and Electrophysiology 2009, 2:345-348.
7. Sanders P, Berenfeld O, Hocini M, Jais P, Vaidyanathan R, Hsu L F, Garrigue S, Takahashi Y, Rotter M, Sacher F, Scavee C, Ploutz-Snyder R, Jalife J, Haïssaguerre M. Spectral analysis identifies sites of highfrequency activity maintaining atrial fibrillation in humans. Circulation 2005, 112:789-797.
8. Botteron G W, Smith J M: A technique for measurement of the extent of spatial organization of atrial activation during atrial fibrillation in the intact human heart. IEEE Trans Biomed Eng 1995, 42:579-586.
9. Botteron G W, Smith J M. Quantitative assessment of the spatial organization of atrial fibrillation in the intact human heart. Circulation 1996, 93:513-518.
10. Ng J, Kadish A H, Goldberger J J. Effect of electrogram characteristics on the relationship of dominant frequency to atrial activation rate in atrial fibrillation. Heart Rhythm 2006, 3:1295-1305.
11. Ng J, Kadish A H, Goldberger J J. Technical considerations for dominant frequency analysis. J Cardiovasc Electrophysiol 2007, 18:757-764.
12. Ng J, Goldberger J J. Understanding and interpreting dominant frequency analysis of electrograms. J Cardiovasc Electrophysiol 2007, 18:680-685.
13. Fischer G, Stühlinger M C, Nowak C N, Wieser L, Tilg B, Hintringer F. On computing dominant frequency from bipolar intracardiac electrograms. IEEE Trans Biomed Eng 2007, 54:165-169.
14. Verma A, Patel D, Famey T, et al. Efficacy of adjuvant anterior left atrial ablation during intracardiac echocardiography-guided pulmonary vein antrum isolation for atrial fibrillation. J Cardiovasc Electrophysiol 2007, 18:151-6.
15. Brooks A G, Stiles M K, Laborderie J, Lau D H, Kuklik P, Shipp N J, Hsu L F, Sanders P. Outcomes of long-standing persistent atrial fibrillation ablation: A systematic review. Heart Rhythm 2010, 7:835-846.
16. Ciaccio E J. Ablation of Longstanding Persistent Atrial Fibrillation. Journal of Atrial Fibrillation 2010, 2:806-809.
17. Ciaccio E J, Biviano A B, Whang W, Wit A L, Garan H, Coromilas J. New methods for estimating local electrical activation rate during atrial fibrillation. Heart Rhythm 2009, 6:21-32.
18. Ciaccio E J, Biviano A B, Whang W, Wit A L, Coromilas J, Garan H. Optimized measurement of activation rate at left atrial sites with complex fractionated electrograms during atrial fibrillation. J Cardiovasc Electrophysiol, 2010, 21:133-143.
19. Narayan S M, Krummen D E, Kahn A M, Karasik P L, Franz M R. Evaluating fluctuations in human atrial fibrillatory cycle length using monophasic action potentials. PACE 2006, 29:1209-1218.
20. Brown J P, Krummen D E, Feld G K, Narayan S M. Using electrocardiographic activation time and diastolic intervals to separate focal from macro-re-entrant atrial tachycardias. J Am Coll Cardiol 2007, 49:1965-1973.
21. Narayan S M, Franz M R. Quantifying fractionation and rate in human atrial fibrillation using monophasic action potentials: Implications for substrate mapping. Europace 2007, 9:vi89-vi95.
22. Stiles M K, Brooks A G, John B; Shashidhar, Wilson L, Kuklik P, Dimitri H, Lau D H, Roberts-Thomson R L, Mackenzie L, Willoughby S, Young G D, Sanders P. The effect of electrogram duration on quantification of complex fractionated atrial electrograms and dominant frequency. J Cardiovasc Electrophysiol. 2008; 19:252-258.
23. Biviano A B, Coromilas J, Ciaccio E J, Whang W, Hickey K, Garan H. Frequency Domain and Time Complex Analyses Manifest Low Correlation and Temporal Variability When Calculating Activation Rates in Atrial Fibrillation Patients. Pacing Clin Electrophysiol. 2011 Jan. 5. doi: 10.1111/j.1540-8159.2010.02993.x. [Epub ahead of print]
24. MacLeod R S, Johnson C R. Map3d: Interactive scientific visualization for bioengineering data. IEEE Engineering Medicine Biology Society 15th Ann Int Conference 1993:30-31.
25. Stridh M, Sörnmo L, Meurling C J, Olsson S B. Sequential characterization of atrial tachyarrhythmias based on ECG time-frequency analysis. IEEE Trans Biomed Eng. 2004, 51:100-114.
26. Ciaccio E J, Drzewiecki G M. Tonometric arterial pulse sensor with noise cancellation. IEEE Trans Biomed Eng. 2008, 55:2388-2396.
27. Stridh M, Bollmann A, Olsson B, Sörnmo L. Detection and feature extraction of atrial tachyarrhythmias. A three stage method of time-frequency analysis. IEEE Engineering Medicine Biology Magazine 2006, 25:31-39.
28. Sandberg F, Stridh M, Sörnmo L. Frequency tracking of atrial fibrillation using hidden markov models. IEEE Trans Biomed Eng 2008, 55:502-511.
29. Ciaccio E J, Coromilas J, Wit A L, Garan H. Onset dynamics of ventricular tachyarrhythmias as measured by dominant frequency. Heart Rhythm 2011 doi:10.1016/j.hrthm.2010.11.028 (in press).
30. Corino V D A, Mainardi L T, Stridh M, Sörnmo L. Improved time-frequency analysis of atrial fibrillation signals using spectral modeling. IEEE Trans Biomed Eng. 2008, 55:2723-2730.
31. Elvan A, Linnenbank A C, van Bemmel M W, Ramdat Misier A R, Delnoy P P H M, Beukema W P, de Bakker J M T. Dominant frequency of atrial fibrillation correlates poorly with atrial fibrillation cycle length. Circulation: AE 2009, 2:634-644.
32. Gray R A, Mornev O A, Jalife J, Aslanidi O V, Pertsov A M. Standing excitation waves in the heart induced by strong alternating electric fields. Phys. Rev. Lett. 2001, 87:168104-1-168104-4.
33. Ciaccio E J, Tennyson C A, Lewis S K, Bhagat G, Green P H. Distinguishing patients with celiac disease by quantitative analysis of videocapsule endoscopy images. Compute Methods Programs Biomed 2010, 100:39-48.
34. Ciaccio E J, Biviano A B, Whang W, Vest J A, Gambhir A, Einstein A J, Garan H. Differences in repeating patterns of complex fractionated left atrial electrograms in longstanding persistent as compared with paroxysmal atrial fibrillation. Circulation: Arrhythmia and Electrophysiology (accepted) 2011.
35. Ciaccio E J, Biviano A B, Whang W, Garan H. Improved Frequency Resolution For Characterization Of Complex Fractionated Atrial Electrograms Frequency Resolution Of Atrial Electrograms.
36. Ciaccio E J, Biviano A B, Whang W, Garan H. Identification Of Recurring Patterns In Fractionated Atrial Electrograms Using New Transform Coefficients. Biomedical Engineering OnLine 2012.
37. Ciaccio E J, Biviano A B, Whang W, Gambhir A, Einstein A J, Garan H. Spectral Profiles Of Complex Fractionated Atrial Electrograms Are Different In Longstanding And Acute Onset Atrial Fibrilation Atrial Electrogram Spectra. Aptara 2012.
38. Pappone C, Rosanio S, Oreto G, Tocchi M, Gugliotta F, Vicedomini G, Salvati A, Dicandia C, Mazzone P, Santinelli V, Gulletta S, Chierchia S. Circumferential radiofrequency ablation of pulmonary vein ostia: A new anatomic approach for curing atrial fibrillation. Circulation. 2000; 102:2619-2628.
39. Pappone C, Rosanio S, Augello G, Gallus G, Vicedomini G, Mazzone P, Gulletta S, Gugliotta F, Pappone A, Santinelli V, Tortoriello V, Sala S, Zangrillo A, Crescenzi G, Benussi S, Alfieri O. Mortality, morbidity, and quality of life after circumferential pulmonary vein ablation for atrial fibrillation: outcomes from a controlled nonrandomized long-term study. J Am Coll Cardiol. 2003; 42:185-197.
40. Oral H, Knight B P, Tada H, Ozaydin M, Chugh A, Hassan S, Scharf C, Lai S W, Greenstein R, Pelosi F Jr, Strickberger S A, Morady F. Pulmonary vein isolation for paroxysmal and persistent atrial fibrillation. Circulation. 2002; 105:1077-1081.
41. Sanders P, Berenfeld O, Hocini M, Jaïs P, Vaidyanathan R, Hsu L F, Garrigue S, Takahashi Y, Rotter M, Sacher F, Scavée C, Ploutz-Snyder R, Jalife J, Haïssaguerre M. Spectral analysis identifies sites of high-frequency activity maintaining atrial fibrillation in humans. Circulation. 2005; 112:789-797.
42. Willems S, Klemm H, Rostock T, Brandstrup B, Ventura R, Steven D, Risius T, Lutomsky B, Meinertz T. Substrate modification combined with pulmonary vein isolation improves outcome of catheter ablation in patients with persistent atrial fibrillation: a prospective randomized comparison. Eur Heart J. 2006; 27:2871-2878.
43. Dixit S, Marchlinski F E, Lin D, Callans D J, Bala R, Riley M P, Garcia F C, Hutchinson M D, Ratcliffe S J, Cooper J M, Verdino R J, Patel V V, Zado E S, Cash N R, Killian T, Tomson T T, Gerstenfeld E P. Randomized ablation strategies for the treatment of persistent atrial fibrillation: RASTA study. Circ Arrhythm Electrophysiol. 2012; 5:287-294.
44. Nademanee K, McKenzie J, Kosar E, Schwab M, Sunsaneewitayakul B, Vasavakul T, Khunnawat C, Ngarmukos T. A new approach for catheter ablation of atrial fibrillation: Mapping of the electrophysiologic substrate. J Am Coll Cardiol 2004; 43:2044-2053.
45. Holm M, Pehrson S, Ingemansson M, Sörnmo L, Johansson R, Sandhall L, Sunemark M, Smideberg B, Olsson C, Olsson S B. Non-invasive assessment of the atrial cycle length during atrial fibrillation in man: introducing, validating and illustrating a new ECG method. Cardiovasc Res. 1998; 38:69-81.
46. Berenfeld O, Mandapati R, Dixit S, Skanes A C, Chen J, Mansour M, Jalife J. Spatially distributed dominant excitation frequencies reveal hidden organization in atrial fibrillation in the Langendorff-perfused sheep heart. J Cardiovasc Electrophysiol. 2000; 11:869-879.
47. Botteron G W, Smith J M. A technique for measurement of the extent of spatial organization of atrial activation during atrial fibrillation in the intact human heart. IEEE Trans Biomed Eng 1995; 42:579-586.
48. Botteron G W, Smith J M. Quantitative assessment of the spatial organization of atrial fibrillation in the intact human heart. Circulation 1996; 93:513-518.
49. Alcaraz R, Sandberg F, Sörnmo L, Rieta J J. Application of frequency and sample entropy to discriminate long-term recordings of paroxysmal and persistent atrial fibrillation. Conf Proc IEEE Eng Med Biol Soc. 2010; 2010:4558-4561.
50. Ciaccio E J, Biviano A B, Whang W, Gambhir A, Garan H. Spectral profiles of complex fractionated atrial electrograms are different in longstanding and acute onset atrial fibrillation. J Cardiovasc Electrophysiol. 2012 May 11. doi: 10.1111/j.1540-8167.2012.02349.x. [Epub ahead of print].
51. Ciaccio E J, Biviano A B, Whang W, Gambhir A, Garan H. Different characteristics of complex fractionated atrial electrograms in acute paroxysmal versus long-standing persistent atrial fibrillation. Heart Rhythm. 2010; 7:1207-1215
52. Ciaccio E J, Biviano A B, Whang W, Vest J A, Gambhir A, Einstein A J, Garan H. Differences in repeating patterns of complex fractionated left atrial electrograms in longstanding persistent atrial fibrillation as compared with paroxysmal atrial fibrillation. Circ Arrhythm Electrophysiol. 2011; 4:470-477.
53. Ciaccio E J, Biviano A B, Whang W, Wit A L, Coromilas J, Garan H. Optimized measurement of activation rate at left atrial sites with complex fractionated electrograms during atrial fibrillation. J Cardiovasc Electrophys 2010; 21:133-143.
54. Ciaccio E J, Biviano A B, Whang W, Coromilas J, Garan H. A new transform for the analysis of atrial fibrillation signals. BioMed Eng OnLine 2011; 10:35-45.
55. Ciaccio E J, Biviano A B, Whang W, Gambhir A, Garan H. Improved frequency resolution for characterization of complex fractionated atrial electrograms. BioMedical Engineering OnLine 2012; 11:17.
56. Stiles M K, Brooks A G, John B; Shashidhar, Wilson L, Kuklik P, Dimitri H, Lau D H, Roberts-Thomson R L, Mackenzie L, Willoughby S, Young G D, Sanders P. The effect of electrogram duration on quantification of complex fractionated atrial electrograms and dominant frequency. J Cardiovasc Electrophysiol. 2008; 19:252-258.
57. Scherr D, Dalal D, Cheema A, Nazarian S, Almasry I, Bilchick K, Cheng A, Henrikson C A, Spragg D, Marine J E, Berger R D, Calkins H, Dong J. Long- and short-term temporal stability of complex fractionated atrial electrograms in human left atrium during atrial fibrillation. J Cardiovasc Electrophysiol. 2009; 20:13-21.
58. Jarman J W E, Wong T, Kojodjojo P, Spohr H, Davies J E, Roughton M, Francis D P, Kanagaratnam P, Markides V, Davies D W, Peters N S. Spatiotemporal behaviour of high dominant frequency during paroxysmal and persistent atrial fibrillation in the human left atrium. circ arrhythm electrophysiol. 2012; CIRCEP.111.967992 published online Jun. 20, 2012.
59. Sanders P, Berenfeld O, Hocini M, Jais P, Vaidyanathan R, Hsu L F, Garrigue S, Takahashi Y, Rotter M, Sacher F, Scavee C, Ploutz-Snyder R, Jalife J, Haissaguerre M. Spectral analysis identifies sites of high-frequency activity maintaining atrial fibrillation in humans. Circulation 2005; 112:789-797.
60. Bongiovanni G, Corsini P, Frosini G, Procedures for computing the discrete fourier transform on staggered blocks. IEEE Transactions Acoustics, Speech, Signal Processing 1976; 24:132-137.
61. Lo P C, Lee Y Y, Real-time implementation of the moving FFT algorithm. Signal Processing 1999; 79:251-259.
62. Nibouche O, Boussakta S, Darnell M, Benaissa M. Algorithms and pipeline architectures for 2-D FFT and FFT-like transforms. Digital Signal Processing 2010; 20:1072-1086.
63. Huang H Y, Lee Y Y, Lo P C. A novel algorithm for computing the 2D split-vector-radix FFT. Signal Processing 2004; 84:561-570.
64. Ciaccio E J, Biviano A B, Whang W, Wit A L, Garan H, Coromilas J. New methods for estimating local electrical activation rate during atrial fibrillation. Heart Rhythm 2009; 6:21-32.
65. Ciaccio E J, Biviano A B, Whang W, Coromilas J, Garan H. A new transform for the analysis of complex fractionated atrial electrograms. Biomed Eng Online 2011; 10:35.
66. Ciaccio E J, Biviano A B, Garan H. Comparison of spectral estimators for characterizing fractionated atrial electrograms. Biomed Eng Online 2013; 12:72.
67. Ciaccio E J, Biviano A B, Garan H. Computational method for high resolution spectral analysis of fractionated atrial electrograms. Computers in Biology and Medicine 2013; 43:1573-1582.
68. Holm M, Pehrson S, Ingemansson M, Sörnmo L, Johansson R, Sandhall L, Sunemark M, Smideberg B, Olsson C, Olsson S B. Non-invasive assessment of the atrial cycle length during atrial fibrillation in man: introducing, validating and illustrating a new ECG method. Cardiovasc Res 1998; 38:69-81.
69. Pehrson S, Holm M, Meurling C, Ingemansson M, Smideberg B, Sörnmo L, Olsson S B. Non-invasive assessment of magnitude and dispersion of atrial cycle length during chronic atrial fibrillation in man. Eur Heart J 1998; 19:1836-1844.
70. Press W H, Teukolsky S A, Vetterling W T, Flannery B P. Numerical Recipes in Fortran, Cambridge University Press, New York (1992) 542-550.
71. Sanders P, Berenfeld O, Hocini M, Jaïs P, Vaidyanathan R, Hsu L F, Garrigue S, Takahashi Y, Rotter M, Sacher F, Scavée C, Ploutz-Snyder R, Jalife J, Haïssaguerre M. Spectral analysis identifies sites of high-frequency activity maintaining atrial fibrillation in humans. Circulation. 2005; 112:789-797.
72. Nademanee K, McKenzie J, Kosar E, Schwab M, Sunsaneewitayakul B, Vasavakul T, Khunnawat C, Ngarmukos T. A new approach for catheter ablation of atrial fibrillation: mapping of the electrophysiologic substrate. J Am Coll Cardiol 2004; 43:2044-2053.
73. Nademanee K, Oketani N. The Role of Complex Fractionated Atrial Electrograms in Atrial Fibrillation Ablation. J Am Coll Cardiol. 2009; 53:790-791.
74. Jarman J W, Wong T, Kojodjojo P, Spohr H, Davies J E, Roughton M, Francis D P, Kanagaratnam P, Markides V, Davies D W, Peters N S. Spatiotemporal behavior of high dominant frequency during paroxysmal and persistent atrial fibrillation in the human left atrium. Circ Arrhythm Electrophysiol 2012; 5:650-658.
75. Narayan S M, Krummen D E, Rappel W J. Clinical mapping approach to diagnose electrical rotors and focal impulse sources for human atrial fibrillation. J Cardiovasc Electrophysiol 2012; 23:447-454.
[1A] Botteron G W, Smith J M: A technique for measurement of the extent of spatial organization of atrial activation during atrial fibrillation in the intact human heart. IEEE Trans Biomed Eng 1995; 42:579-586.
[2A] Botteron G W, Smith J M: Quantitative assessment of the spatial organization of atrial fibrillation in the intact human heart. Circulation 1996; 93:513-518.
[3A] Fischer G, Stühlinger M C, Nowak C N, Wieser L, Tilg B, Hintringer F: On computing dominant frequency from bipolar intracardiac electrograms. IEEE Trans Biomed Eng 2007; 54:165-169.
[4A] Ng J, Kadish A H, Goldberger J J: Effect of electrogram characteristics on the relationship of dominant frequency to atrial activation rate in atrial fibrillation. Heart Rhythm 2006; 3:1295-1305.
[5A] Ng J, Kadish A H, Goldberger J J. Technical considerations for dominant frequency analysis. J Cardiovasc Electrophysiol 2007; 18:757-764.
[6A] Ciaccio E J, Biviano A B, Whang W, Wit A L, Garan H, Coromilas J: New methods for estimating local electrical activation rate during atrial fibrillation. Heart Rhythm 2009; 6:21-32.
[7A] Ciaccio E J, Biviano A B, Whang W, Wit A L, Coromilas J, Garan H. Optimized measurement of activation rate at left atrial sites with complex fractionated electrograms during atrial fibrillation. J Cardiovasc Electrophysiol. 2010; 21:133-143.
[8A] Ciaccio E J, Tennyson C A, Lewis S K, Krishnareddy S, Bhagat G, Green P H. Distinguishing patients with celiac disease by quantitative analysis of videocapsule endoscopy images. Comput Methods Programs Biomed. 2010b; 100:39-48.
[9A] Green P H. Mortality in celiac disease, intestinal inflammation, and gluten sensitivity. JAMA 2009; 302:1225-1226.
[10A] Tennyson C A, Lewis S K, Green P H. New and developing therapies for celiac disease. Therap Adv Gastroenterol. 2009; 2:303-309.
[11A] Rey J F, Repici A, Kuznetsov K, Boyko V, Aabakken L. Optimal preparation for small bowel examinations with video capsule endoscopy. Dig Liver Dis. 2009; 41:486-493.
[12A] Triantafyllou K. Can we improve the diagnostic yield of small bowel video-capsule endoscopy? World J Gastrointest Enclose. 2010; 2:143-146.
[13A] Vilarino, F. Spyridonos, P. Pujol, O. Vitria, J. Radeva, P. de Iorio, F. Automatic Detection of Intestinal Juices in Wireless Capsule Video Endoscopy. Pattern Recognition, 2006. 18th International Conference, Hong Kong; ICPR 2006; 4:719-722.
[14A] Vilarino F, Spyridonos P, Deiorio F, Vitria J, Azpiroz F, Radeva P. Intestinal motility assessment with video capsule endoscopy: automatic annotation of phasic intestinal contractions. IEEE Trans Med Imaging. 2010; 29:246-259.
[15A] Gheorghe C, Jacob R, Bancila I. Olympus capsule endoscopy for small bowel examination. J Gastrointestin Liver Dis, 2007; 16:309-313.
[16A] Press W H, Flannery B P, Teukolsky S A, Vetterling W T, Numerical Recipes in C: The Art of Scientific Computing, 2nd ed. New York, N.Y.: Cambridge Univ. Press, 1992, p 501-502.
[17A] Frigo M, Johnson S G. The design and implementation of FFTW3. Proc. IEEE 2005; 93:216-231.
[18A] Ciaccio E J, Tennyson C A, Bhagat G, Lewis S K, Green P H. Classification of videocapsule endoscopy image patterns: comparative analysis between patients with celiac disease and normal individuals. Biomed Eng Online 2010c; 9:44-55.
[19A] Green P H. Celiac disease: how many biopsies for diagnosis? Gastrointest Endosc. 2008; 67:1088-1090.
[20A] Vécsei A, Fuhrmann T, Liedlgruber M, Brunauer L, Payer H, Uhl A. Automated classification of duodenal imagery in celiac disease using evolved Fourier feature vectors. Comput Methods Programs Biomed. 2009 August; 95(2 Suppl):568-78,
[21A] Charisis, V.; Hadjileontiadis, L. J.; Liatsos, C. N.; Mavrogiannis, C. C.; Sergiadis, G. D. Abnormal pattern detection in Wireless Capsule Endoscopy images using nonlinear analysis in RGB color space. Engineering in Medicine and Biology Society (EMBC), 2010 Annual International Conference of the IEEE Aug. 31, 2010-Sep. 4, 2010 Buenos Aires. Conf Proc IEEE Eng Med Biol Soc. 2010; 1:3674-7.
[22A] Metzger Y C, Adler S N, shitrit A B, Koslowsky B, Bjarnason I. Comparison of a new PillCam™ SB2 video capsule versus the standard PillCam™ SB for detection of small bowel disease. Reports in Medical Imaging 2009; 2:7-11.
[23A] Culliford A, Daly J, Diamond B, Rubin M, Green P H. The value of wireless capsule endoscopy in patients with complicated celiac disease. Gastrointest Endosc. 2005; 62:55-61.
[24A] Green P H, Rubin M. Capsule endoscopy in celiac disease. Gastrointest Endosc. 2005; 62:797-799.
[25A] Green P H, Rubin M. Capsule endoscopy in celiac disease: diagnosis and management (Review). Gastrointest Endosc Clin N Am. 2006; 16:307-316.
[26A] Seguí S, Igual L, Radeva P, Malagelada C, Azpiroz F Vitria J. A semi-supervised learning method for motility disease diagnostic. Progress in Pattern Recognition, Image Analysis and Applications Lecture Notes in Computer Science, 2007; 4756:773-782.
[27A] Seguí S, Igual L, Vilarino F, Radeva P, Malagelada C, Azpiroz F, Vitrià J. Diagnostic system for intestinal motility disfunctions using video capsule endoscopy. Computer Vision Systems Lecture Notes in Computer Science, 2008; 5008:251-260.

What is claimed is:

1. A non-transitory computer-accessible medium having stored thereon computer-executable instructions for generating information associated with at least one of at least one signal or data received from at least one structure, wherein, when a computer arrangement executes the instructions, the computer arrangement is configured to perform procedures comprising:
determining at least one basis based on a combination of a plurality of portions of at least one of the at least one signal or the data; and
generating the information as a function of the at least one basis.

2. The computer readable medium of claim 1, wherein the combination includes at least one of a summation, an average, a weighted average, or a statistical representation.

3. The computer readable medium of claim 2, wherein the summation includes a summation of a plurality of segments of the at least one signal or the data.

4. The computer readable medium of claim 1, wherein the generation of the information comprises applying a transform.

5. The computer readable medium of claim 4, wherein the transform relates a summation to at least one frequency of the at least one signal so as to generate a power spectrum.

6. The computer readable medium of claim 4, wherein the computer arrangement is further configured to quantify at least one characteristic associated with the at least one signal or the data based on the transform.

7. The computer readable medium of claim 4, wherein the computer arrangement is further configured to cause a recognition of a source pattern of the at least one signal or the data based on the transform

8. The computer readable medium of claim 4, wherein the transform is a spectral estimator.

9. The computer readable medium of claim 8, wherein the computer arrangement is further configured to generate the information substantially in real time using the spectral estimator.

10. The computer readable medium of claim 9, wherein the spectral estimator is integrated into a circuit board.

11. The computer readable medium of claim 1, wherein the at least one signal or the data includes at least one of a video-capsule image.

12. The computer readable medium of claim 10, wherein the at least one video-capsule image is associated with at least one of a celiac disease or a cardiac signal as obtained during atrial fibrillation.

13. The computer readable medium of claim 1, wherein the information includes at least one of a dominant frequency, a dominant period, a mean, a standard deviation in a power spectral profile, or a further statistical representation.

14. The computer readable medium of claim 1, wherein the computer arrangement is further configured to reduce at least one of a noise, an interference, or an artifact during the generation of the information.

15. The computer readable medium of claim 1, wherein the computer arrangement is further configured to generate a frequency resolution for a given time period of the at least one signal or the data.

16. The computer readable medium of claim 1, wherein the signal or the data includes at least one image.

17. The computer readable medium of claim 1, wherein the combination includes an average of at least one first segment and at least one second segment of the at least one signal or the data, wherein the at least one second segment is adjacent to the at least one first segment.

18. The computer readable medium of claim 1, wherein the combination includes an average of a plurality of segments of varying segment lengths of the at least one signal or the data.

19. A method for generating information associated with at least one of at least one signal or data received from at least one structure, comprising:
determining at least one basis based on a combination of a plurality of portions of at least one of the at least one signal or the data; and
using a programmed computer hardware arrangement, generating the information as a function of the at least one basis.

20. A system for generating information associated with at least one of at least one signal or data received from at least one structure, comprising:
a computer arrangement configured to:
determine at least one basis based on a combination of a plurality of portions of at least one of the at least one signal or the data, and
generate information as a function of the at least one basis.
